(19) United States
(12) Patent Application Publication
(10) Pub. No.: US 2023/0201268 A1
(43) Pub. Date: Jun. 29, 2023

(54) TREATMENT OF CANCER BY ADMINISTRATION OF NEURONS AND DERIVATIVES THEREOF

(71) Applicant: James Veltmeyer, La Jolla, CA (US)

(72) Inventor: James Veltmeyer, La Jolla, CA (US)

(21) Appl. No.: 18/083,043

(22) Filed: Dec. 16, 2022

Related U.S. Application Data
(60) Provisional application No. 63/293,742, filed on Dec. 24, 2021.

(51) Int. Cl. A61K 35/30; A61K 35/28; A61P 35/00; A61P 17/00

(52) U.S. Cl. CPC A61K 35/30; A61K 35/28; A61P 35/00; A61P 17/00; A61K 2035/124

(57) ABSTRACT

Disclosed are means, methods and compositions useful for the prevention, inhibition, and regression of a neoplastic growth through altering the tumor microenvironment using neurons, derivatives from neurons, and neuromodulating agents and/or energy-based approaches. In one embodiment allogeneic neuronal cells are generated from pluripotent stem cells and administered locally in tumor microenvironment to induce an alteration tumor growth directly or in combination with other therapeutic approaches. In some embodiments exosomes from allogeneic neuronal cells are administered as a therapeutic agent. In other embodiments of the invention re-innervation of tumor tissue and/or alteration of neurons innervating said tumor issue is performed by administration of neurotrophic factors.

### CROSS-REFERENCE TO RELATED APPLICATIONS

This application claims priority to U.S. Provisional Application No. 63/293,742, titled “Treatment of Cancer by Administration of Neurons and Derivatives Thereof”, and filed Dec. 24, 2021, which is incorporated by reference in its entirety.

### FIELD OF THE INVENTION

The teachings herein are directed to compositions and methods for treating cancer by administration of neurons and derivatives thereof.

### BACKGROUND OF THE INVENTION

In recent years there has been a growing realization that immune responses play a central role in cancer biology by eliminating many tumours at a very early stage and keeping those that avoid total elimination in a state of equilibrium, sometimes for many years. The eventual escape from this equilibrium phase with clinical manifestation of the disease is associated with dysregulated immune responses, manifesting, for example, as chronic inflammation or immunesuppression. The strong and increasing evidence that the immune system is critically involved in the development, structural nature and progression of cancer has led to renewed interest in immunotherapeutic strategies for treatment of this class of diseases. To date, most attempts to develop such strategies have been based on the use of antigens derived from the patient's own tumour or from tumour cell lines and the transfer of ex-vivo expanded populations of tumour antigen-specific cytotoxic cells and antigen-presenting cells.

Cancer has been associated with inflammation since 1863, when Rudolf Virchow discovered leucocytes in neoplastic tissues and so made the first connection between inflammation and cancer. Since then, chronic inflammation has been deemed to be a risk factor for cancer. These reports demonstrate that an inflammatory environment supports tumour development and is consistent with that observed at tumour sites. However, the relationship of cancer with inflammation is not limited to the onset of the disease due to chronic inflammation. Schwartsburd proposed that chronic inflammation occurs due to tumour environment stress and that this generates a shield from the immune system. It has been recently demonstrated that the tumour microenvironment resembles an inflammation site, with significant support for tumour progression, through chemokines, cytokines, lymphocytes and macrophages which contribute to both the neovascularisation and vasal dilation for increased blood flow, the immunosuppression associated with the malignant disease, and the establishment of tumour metastasis. Furthermore, this inflammation-site tumour-generated microenvironment, apart from its significant role in protection from the immune system and promotion of cancer progression, has an adverse effect on the success of current cancer treatments. Indeed, it has been found that the inflammatory response in cancer can compromise the pharmacodynamics of chemotherapeutic agents.

Moreover, metastatic cancer cells leave the tumour as microcolonies, containing lymphocytes and platelets as well as tumour cells. Inflammation continues to play a role at metastatic sites by creating a cytokine milieu conducive to tumour growth. Immune homeostasis consists of a tightly regulated interplay of pro- and anti-inflammatory signals. For example, loss of the anti-inflammatory signals leads to chronic inflammation and proliferative signalling. Interestingly, cytokines that both promote and suppress proliferation of the tumour cells are produced at the tumour site. As in the case of cancer initiation, it is the imbalance between the effects of these various processes that results in tumour promotion.

It is believed that, to treat cancer, the most effective type of immune response is of a Type 1, which favours the induction of CD4+ Th1 cellular responses, and of CD8+ CTL responses. In the context of cancer vaccines, many immune stimulants are used, which promote the development of Th1 responses and are thought to inhibit the production of a Th2 response. To date, a major barrier to attempts to develop effective immunotherapy for cancer has been an inability to break immunosuppression at the cancer site and restore normal networks of immune reactivity. The physiological approach of immunotherapy is to normalize the immune reactivity so that the endogenous tumour antigens would be again recognized and effective cytolytic responses would be developed against cells bearing these antigens. Anti-cancer immune responses accompanying the action of chemo- and radiotherapy have been recently reviewed and show that such responses are critical to therapeutic success by eliminating residual cancer cells and maintaining micrometastases in a state of dormancy. However, this reference makes it clear that there is no simple immunotherapeutic strategy available for consistently enhancing such immune responses. There is evidence that therapeutic procedures that induce certain forms of immunogenic cancer cell death also lead to release of tumour antigens. There are three main types of cell death (Tesniere et al, Cell Death Differ 2008; 15:3-12): apoptosis (type 1), autophagy (type 2) and necrosis (type 3). Apoptosis, or programmed cell death, is a common and regular occurring phenomenon essential for tissue remodelling, especially in utero but also ex utero. It is characterized by DNA fragmentation in the nucleus and condensation of the cytoplasm to form ‘apoptotic bodies’ which are engulfed and digested by phagocytic cells. In autophagy, cell organelles and cytoplasm are sequestered in vacuoles which are extruded from the cell. Although this provides a means of survival for cells in adverse nutritional conditions or other stressful situations, excess autophagy results in cell death. Necrosis is a ‘cruder’ process characterized by damage to intracellular organelles and cell swelling, resulting in rupture of the cell membrane and release of intracellular material.

Studies of adoptive T cell immunotherapy [1] along with recently reported positive clinical results in non-Hodgkins lymphoma [2, 3] and prostate cancer immunotherapy targeting tumor-associated antigens (TAAs) have provided proof of concept that the immune system can support a clinically effective anti-tumor immune response. Although the benefits of anti-tumor immunotherapy has not been demonstrated in a wide range of tumor types, it has been postulated that the missing critical element is a sufficiently potent, readily translatable cancer vaccine strategy [4]. Patients with breast cancer can have endogenous or immunotherapy elicited humoral and cellular responses to several tumor-associated antigens (TAAs) [5-14], however, these have generally been of sub-optimal magnitude with elusive clinical efficacy. Additionally, breast cancer patients with significant inflammatory infiltrates, i.e. medullary breast carcinoma, have significantly improved survival despite greater cellular anaplasia[15-17]. Thus, it is reasonable to hypothesize that a sufficiently potent, antigen-specific immunotherapeutic strategy for breast cancer would have clinical efficacy and offer a valuable new treatment modality.

A variety of TAAs have been identified in breast cancer consisting of overexpressed normal proteins and mutated proteins that are normally found in breast tissue, however, only a minority of the TAAs that have been discovered so far are immunogenic, which limits the potential use for immunotherapy. In addition, while the overwhelming majority of TAAs are expressed in tumor cells, they are typically also expressed in a variety of normal cells, e.g. the breast cancer TAAs; epidermal growth factor receptors (HER2), carcinoembryonic antigen (CEA), mucin (MUC1), the tumor suppressor protein p53, and telomerase reverse transcriptase (TERT). Thus, they are recognized by the immune system as self-molecules, and the immune system has protective mechanisms for preventing recognition of self-tissue antigens and autoimmune responses. Additionally, tumors employ other mechanisms for escaping immune surveillance, such as: (i) low level expression of MHC class I molecules [18]; (ii) lack of expression of B7 (CD80/CD86) co-stimulatory molecules [19]; (iii) production of cytokines that stimulate the accumulation of immune-suppressor cells [20, 21]; and (iv) ineffective processing and presentation of self-antigens by “professional” antigen-presenting cells (APC) [22]. This probably explains why TAA or tumor cell vaccines that have been used in clinical trials generally do not induce strong protective immunity [1]. Identification of novel TAA that are not expressed on normal cells may provide an attractive alternative particularly if combined with potent immunotherapeutic platforms, because these antigens are less likely to be subject to the tolerogenic mechanisms that limit immune responses to “self” antigens and therefore, may be better immunogens. [23]

Approximately 211,000 new cases of breast cancer are diagnosed yearly and nearly 41,000 deaths are expected due to this malignancy [24]. Although, recent treatment advances for breast cancer have led to increased disease free and overall survival, the majority of patients recur and survival curves show no signs of a plateau associated with disease cure, indicating persistence of occult microscopic disease that ultimately leads to morbidity and mortality. The immune system is uniquely suited to “search out and destroy” this occult disease, thus providing a basis for the promise of anti-tumor immunotherapy.

Ductal carcinoma in situ (DCIS) is the most common type of non-invasive breast cancer. Ductal implies that the cancer starts inside the milk ducts and in situ means “in its original place.” The diagnosis of DCIS comes with approximately 30% chance of recurrence within 5-10 years. Women who have breast-conserving surgery (lumpectomy) for DCIS without radiation therapy have about a 25% to 30% chance of having a recurrence at some point in the future. However the inclusion of radiation therapy in the treatment plan after surgery drops the risk of recurrence to about 15%. If breast cancer does come back after earlier DCIS treatment, the recurrence is non-invasive (DCIS again) about half the time and invasive about half the time. According to the American Cancer Society, about 60,000 cases of DCIS are diagnosed in the United States each year, accounting for about 1 out of every 5 new breast cancer cases [25]. The presence of Her2/neu is known to predict higher levels of recurrence in DCIS [26]. Accordingly, assessment of immunotherapy such as DC administration in this condition post surgically may be more intellectually appealing since the surgical debulking of the tumor would allow an escape from immune suppression of the tumor.

Czerniecki and colleagues treated 13 HER-2/neu expressing DCIS with 4 weekly intranodal administrations of DC pulsed with HER-2/neu HLA class I and II peptides. Before vaccination, many subjects possessed HER-2/neu-HLA-A2 tetramer-staining CD8(pos) T cells that expressed low levels of CD28 and high levels of the inhibitory B7 ligand CTLA-4, but this ratio inverted after vaccination. The vaccinated subjects also showed high rates of peptide-specific sensitization for both IFN-gamma-secreting CD4(pos) (85%) and CD8(pos) (80%) T cells, with recognition of antigenically relevant breast cancer lines, accumulation of T and B lymphocytes in the breast, and induction of complement-dependent, tumor-lytic antibodies. Seven of 11 evaluable patients also showed markedly decreased HER-2/neu expression in surgical tumor specimens, often with measurable decreases in residual DCIS. The vaccine was well tolerated, however recurrence data was not provided [27]. Despite this, the study provided some evidence that immune modulation is feasible in this patient subset. A subsequent report by the same group examined a larger number of patients. Specifically, 27 HER-2/neu expressing DCIS patients were treated with DC1-polarized dendritic cells (DC1) pulsed with 6 HER-2/neu peptide pulsed DC into groin lymph nodes 4 times at weekly intervals before scheduled surgical resection of ductal carcinoma in situ. Sensitization of T cells to at least 1 class II peptide in 22 of 25 evaluable patients was observed. Additionally 11 of 13 HLA-A2.1 patients were successfully sensitized to class I peptides. Responses to the peptide in terms of proliferation and interferon gamma production were observed up to 52-month postimmunization [28]. A clinical analysis of the same patients reported no adverse events and reduction or eradication of Her2/neu in surgical biopsy samples [29]. Immune response induction was not associated with estrogen receptor status, as reported in a follow-up publication [30].

In advanced cancer patients, Brossart et al reported feasibility of inducing immune response with DC. They treated 10 patients with advanced breast and ovarian cancer using DC pulsed with HER-2/neu- or MUC1-derived peptides, respectively. The DC vaccinations were well tolerated with no side effects. In 5 of 10 patients, peptide-specific cytotoxic T lymphocytes (CTLs) could be detected in the peripheral blood using both intracellular IFN-gamma staining and (51)Cr-release assays. The major CTL response in vivo was induced with the HER-2/neu-derived E75 and the MUC1-derived M1.2 peptide, which lasted for more than 6 months. In addition, in one patient vaccinated with the MUC1-derived peptides, CEA- and MAGE-3 peptide-specific T-cell responses were detected after several vaccinations. In a second patient immunized with the HER-2/neu peptides, MUC1-specific T lymphocytes were induced after 7 immunizations, suggesting that epitope spreading in vivo might occur after successful immunization with a single tumor antigen [31].

Morse et al performed two small studies to test the safety, feasibility, and immunologic and clinical responses to immunizations with in vitro-generated DCs loaded with either a human leukocyte antigen A2-restricted peptide fragment of the extracellular domain of the tumor antigen HER2 (E75) or a HER2 intracellular domain (ICD) protein in patients with high-risk resected breast cancer or metastatic cancers expressing HER2. No toxicities due to the immunizations in any of the patients. In the study of DCs loaded with the E75 peptide, 1 of 6 patients with metastatic HER2-expressing malignancies who completed all immunizations had stable disease for 6 months; the remainder of the patients had progressive disease. Delayed-type hypersensitivity (DTH) reactivity (2-3 mm of induration) at E75-loaded DC injection sites was observed in 2 of 5 patients evaluated but was similar at the unloaded DC injection sites. In 2 patients, the DTH sites underwent biopsy and a perivascular infiltrate of CD4 and CD8 cells was demonstrated, which was greater in the E75-loaded DC injection sites than in the unloaded DC sites. In the pilot study of ICD-loaded DC in patients with high-risk resected breast cancer, all 3 patients enrolled had no evidence of recurrence at a follow-up of up to 2.5 years. Intracellular domain-specific T-cell responses were detected directly from the peripheral blood by enzyme-linked immunospot and proliferation assay in 2 patients [32]. Other studies have been performed using peptide pulsed or lysate pulsed DC for advanced breast cancer. All studies demonstrated some level of immune response induction, however none were powered to demonstrate statistically significant tumor regression rates [33-35]

### SUMMARY

Preferred embodiments are directed to methods of treating cancer and/or overcoming cancer associated immune suppression comprising the steps of: a) selecting a patient suffering from a tumor; b) administering to said patient a neuronal population growth ex vivo; and c) optionally providing one or more therapeutic interventions.

Preferred methods include embodiments, wherein said patient is selected based on amount of cellular infiltration into said tumor.

Preferred methods include embodiments, wherein said cellular infiltration comprises tumor infiltrating lymphocytes.

Preferred methods include embodiments, wherein patients are stratified based on amount of tumor infiltrating T cells.

Preferred methods include embodiments, wherein patients are stratified based on amount of tumor infiltrating CD4 T cells.

Preferred methods include embodiments, wherein patients are stratified based on amount of tumor infiltrating CD4 Th1 cells.

Preferred methods include embodiments, wherein patients possessing more that 1 T cell per 100,000 tumor cells and possessing ability of said T cells to proliferate are selected for therapy.

Preferred methods include embodiments, wherein said T cell proliferation is induced by an agent selected from the group consisting of: a) a mitogen; b) a cytokine; c) a molecule capable of activating T cell receptor in an antigen nonspecific manner; and d) a molecule capable of activating T cell receptor in an antigen specific manner.

Preferred methods include embodiments, wherein said mitogen is conconavalin-A.

Preferred methods include embodiments, wherein said mitogen is phytohemagluttinin.

Preferred methods include embodiments, wherein said mitogen is pokeweed mitogen.

Preferred methods include embodiments, wherein said mitogen is Galanthus nivalis Lectin.

Preferred methods include embodiments, wherein said cytokine is interleukin-1 beta.

Preferred methods include embodiments, wherein said cytokine is interleukin-2.

Preferred methods include embodiments, wherein said cytokine is interleukin-3.

Preferred methods include embodiments, wherein said cytokine is interleukin-4.

Preferred methods include embodiments, wherein said cytokine is interleukin-7.

Preferred methods include embodiments, wherein said cytokine is interleukin-10.

Preferred methods include embodiments, wherein said cytokine is interleukin-12.

Preferred methods include embodiments, wherein said cytokine is interleukin-18.

Preferred methods include embodiments, wherein said cytokine is interleukin-17.

Preferred methods include embodiments, wherein said cytokine is interleukin-20.

Preferred methods include embodiments, wherein said cytokine is interleukin-35.

Preferred methods include embodiments, wherein said cytokine is interferon alpha.

Preferred methods include embodiments, wherein said cytokine is interferon beta.

Preferred methods include embodiments, wherein said cytokine is interferon gamma.

Preferred methods include embodiments, wherein said cytokine is interferon omega.

Preferred methods include embodiments, wherein said molecule capable of activating said TCR in an antigen non-specific manner is an antibody.

Preferred methods include embodiments, wherein said antibody is capable of ligating TCR.

Preferred methods include embodiments, wherein said antibody is capable of ligating CD3.

Preferred methods include embodiments, wherein said antibody is OKT3.

Preferred methods include embodiments, wherein said molecule capable of activating said TCR in an antigen non-specific manner is a microbody.

Preferred methods include embodiments, wherein said molecule capable of activating said TCR in an antigen non-specific manner is a bispecific antibody.

Preferred methods include embodiments, wherein said molecule capable of activating said TCR in an antigen non-specific manner is a recombinant protein.

Preferred methods include embodiments, wherein said molecule capable of activating said TCR in an antigen non-specific manner is an aptamer.

Preferred methods include embodiments, wherein said molecule capable of activating said TCR in an antigen non-specific manner is a DNano particle.

Preferred methods include embodiments, wherein said molecule capable of activating said TCR in an antigen specific manner is an autoantigen.

Preferred methods include embodiments, wherein said cancer associated immune suppression is inhibition of T cell cytotoxic activity.

Preferred methods include embodiments, wherein said T cell cytotoxic activity is mediated by Fas Ligand.

Preferred methods include embodiments, wherein said T cell cytotoxic activity is mediated by TRAIL.

Preferred methods include embodiments, wherein said T cell cytotoxic activity is mediated by perforin.

Preferred methods include embodiments, wherein said T cell cytotoxic activity is mediated by granzyme A.

Preferred methods include embodiments, wherein said T cell cytotoxic activity is mediated by granzyme B.

Preferred methods include embodiments, wherein said T cell cytotoxic activity is mediated by TNF-alpha.

Preferred methods include embodiments, wherein said T cell cytotoxic activity is mediated by TNF-beta.

Preferred methods include embodiments, wherein said T cell cytotoxic activity is mediated by induction of apoptosis.

Preferred methods include embodiments, wherein said T cell cytotoxic activity is mediated by induction of necrosis.

Preferred methods include embodiments, wherein said T cell cytotoxic activity is mediated by pyroptosis.

Preferred methods include embodiments, wherein said T cell cytotoxic activity is mediated by necroptosis.

Preferred methods include embodiments, wherein said T cell cytotoxic activity is mediated by alternation in mitochondrial membrane potential in the target cell

Preferred methods include embodiments, wherein said cancer induced immune is suppression of cytokine production.

Preferred methods include embodiments, wherein said cytokine production is induced by an agent selected from one or more agents from a group comprising of: a) a mitogen; b) a cytokine; c) a molecule capable of activating T cell receptor in an antigen nonspecific manner; and d) a molecule capable of activating T cell receptor in an antigen specific manner.

Preferred methods include embodiments, wherein said mitogen is conconavalin-A.

Preferred methods include embodiments, wherein said mitogen is phytohemagluttinin.

Preferred methods include embodiments, wherein said mitogen is pokeweed mitogen.

Preferred methods include embodiments, wherein said mitogen is Galanthus nivalis Lectin.

Preferred methods include embodiments, wherein said cytokine is interleukin-1 beta.

Preferred methods include embodiments, wherein said cytokine is interleukin-2.

Preferred methods include embodiments, wherein said cytokine is interleukin-3.

Preferred methods include embodiments, wherein said cytokine is interleukin-4.

Preferred methods include embodiments, wherein said cytokine is interleukin-7.

Preferred methods include embodiments, wherein said cytokine is interleukin-10.

Preferred methods include embodiments, wherein said cytokine is interleukin-12.

Preferred methods include embodiments, wherein said cytokine is interleukin-18.

Preferred methods include embodiments, wherein said cytokine is interleukin-17.

Preferred methods include embodiments, wherein said cytokine is interleukin-20.

Preferred methods include embodiments, wherein said cytokine is interleukin-35.

Preferred methods include embodiments, wherein said cytokine is interferon alpha.

Preferred methods include embodiments, wherein said cytokine is interferon beta.

Preferred methods include embodiments, wherein said cytokine is interferon gamma.

Preferred methods include embodiments, wherein said cytokine is interferon omega.

Preferred methods include embodiments, wherein said molecule capable of activating said TCR in an antigen non-specific manner is an antibody.

Preferred methods include embodiments, wherein said antibody is capable of ligating TCR.

Preferred methods include embodiments, wherein said antibody is capable of ligating CD3.

Preferred methods include embodiments, wherein said antibody is OKT3.

Preferred methods include embodiments, wherein said molecule capable of activating said TCR in an antigen non-specific manner is a microbody.

Preferred methods include embodiments, wherein said molecule capable of activating said TCR in an antigen non-specific manner is a bispecific antibody.

Preferred methods include embodiments, wherein said molecule capable of activating said TCR in an antigen non-specific manner is a recombinant protein.

Preferred methods include embodiments, wherein said molecule capable of activating said TCR in an antigen non-specific manner is an aptamer.

Preferred methods include embodiments, wherein said molecule capable of activating said TCR in an antigen non-specific manner is a DNano particle.

Preferred methods include embodiments, wherein said molecule capable of activating said TCR in an antigen specific manner is an autoantigen.

Preferred methods include embodiments, wherein said cancer is treated by administration of neuronal or neuronal-like cells.

Preferred methods include embodiments, wherein said neuronal or neuronal-like cells are administered intratumorally.

Preferred methods include embodiments, wherein said neuronal or neuronal-like cells are administered in an immature state.

Preferred methods include embodiments, wherein said immature neuronal or neuronal-like cells are allowed to differentiate intratumorally.

Preferred methods include embodiments, wherein said immature neuronal or neuronal-like cells are generated from hematopoietic stem cells.

Preferred methods include embodiments, wherein said hematopoietic stem cell is capable of generating leukocytic, lymphocytic, thrombocytic and erythrocytic cells when transplanted into an immunodeficient animal.

Preferred methods include embodiments, wherein said hematopoietic stem cell is non-adherent to plastic.

Preferred methods include embodiments, wherein said hematopoietic stem cell is adherent to plastic.

Preferred methods include embodiments, wherein said hematopoietic stem cell is exposed to hyperthermia.

Preferred methods include embodiments, wherein said hematopoietic stem cell expresses interleukin-3 receptor.

Preferred methods include embodiments, wherein said hematopoietic stem cell expresses interleukin-1 receptor.

Preferred methods include embodiments, wherein said hematopoietic stem cell expresses c-met.

Preferred methods include embodiments, wherein said hematopoietic stem cell expresses mpl.

Preferred methods include embodiments, wherein said hematopoietic stem cell expresses interleukin-11 receptor.

Preferred methods include embodiments, wherein said hematopoietic stem cell expresses G-CSF receptor.

Preferred methods include embodiments, wherein said hematopoietic stem cell expresses GM-CSF receptor.

Preferred methods include embodiments, wherein said hematopoietic stem cell expresses M-CSF receptor.

Preferred methods include embodiments, wherein said hematopoietic stem cell expresses VEGF-receptor.

Preferred methods include embodiments, wherein said hematopoietic stem cell expresses c-kit.

Preferred methods include embodiments, wherein said hematopoietic stem cell expresses CD33.

Preferred methods include embodiments, wherein said hematopoietic stem cell expresses CD133.

Preferred methods include embodiments, wherein said hematopoietic stem cell expresses CD34.

Preferred methods include embodiments, wherein said hematopoietic stem cell expresses Fas ligand.

Preferred methods include embodiments, wherein said hematopoietic stem cell does not express lineage markers.

Preferred methods include embodiments, wherein said hematopoietic stem cell does not express CD14.

Preferred methods include embodiments, wherein said hematopoietic stem cell does not express CD16.

Preferred methods include embodiments, wherein said hematopoietic stem cell does not express CD3.

Preferred methods include embodiments, wherein said hematopoietic stem cell does not express CD56.

Preferred methods include embodiments, wherein said hematopoietic stem cell does not express CD38.

Preferred methods include embodiments, wherein said hematopoietic stem cell does not express CD30.

Preferred methods include embodiments, wherein said neuronal or neuronal like immature cells are generated from mesenchymal stem cells.

Preferred methods include embodiments, wherein said mesenchymal stem cells are naturally occurring mesenchymal stem cells.

Preferred methods include embodiments, wherein said mesenchymal stem cells are generated in vitro.

Preferred methods include embodiments, wherein said naturally occurring mesenchymal stem cells are tissue derived.

Preferred methods include embodiments, wherein said naturally occurring mesenchymal stem cells are derived from a bodily fluid.

Preferred methods include embodiments, wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments, wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods include embodiments, wherein said mesenchymal stem cells are plastic adherent.

Preferred methods include embodiments, wherein said mesenchymal stem cells express a marker selected from a group comprising of: a) CD73; b) CD90; and c) CD105.

Preferred methods include embodiments, wherein said mesenchymal stem cells lack expression of a marker selected from a group comprising of: a) CD14; b) CD45; and c) CD34.

Preferred methods include embodiments, wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments, wherein said mesenchymal stem cells from umbilical cord tissue do not express markers selected from a group comprising of: a) CD117; b) CD31; c) CD34; and CD45;

Preferred methods include embodiments, wherein said mesenchymal stem cells from umbilical cord tissue express, relative to a human fibroblast, increased levels of interleukin 8 and reticulon 1

Preferred methods include embodiments, wherein said mesenchymal stem cells from umbilical cord tissue have the potential to differentiate into cells of at least a skeletal muscle, vascular smooth muscle, pericyte or vascular endothelium phenotype.

Preferred methods include embodiments, wherein said mesenchymal stem cells from umbilical cord tissue express markers selected from a group comprising of: a) CD10; b) CD13; c) CD44; d) CD73; and e) CD90.

Preferred methods include embodiments, wherein said umbilical cord tissue mesenchymal stem cell is an isolated umbilical cord tissue cell isolated from umbilical cord tissue substantially free of blood that is capable of self-renewal and expansion in culture,

Preferred methods include embodiments, wherein said umbilical cord tissue mesenchymal stem cells has the potential to differentiate into cells of other phenotypes.

Preferred methods include embodiments, wherein said other phenotypes comprise: a) osteocytic; b) adipogenic; and c) chondrogenic differentiation.

Preferred methods include embodiments, wherein said cord tissue derived mesenchymal stem cells can undergo at least 20 doublings in culture.

Preferred methods include embodiments, wherein said cord tissue derived mesenchymal stem cell maintains a normal karyotype upon passaging

Preferred methods include embodiments, wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods include embodiments, wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments, wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

Preferred methods include embodiments, wherein said umbilical cord tissue derived cells express markers selected from a group comprising of: a) TRA1-60; b) TRA1-81; c) SSEA3; d) SSEA4; and e) NANOG.

Preferred methods include embodiments, wherein said umbilical cord tissue-derived cells are positive for alkaline phosphatase staining.

### BRIEF DESCRIPTION OF THE DRAWINGS

FIG. 1 is a bar graph showing NGF resulted in inhibition of IL-10 induced suppression of NK cell activity.

FIG. 2 is a bar graph showing NGF resulted in inhibition of VEGF induced suppression of NK cell activity

FIG. 3 is a bar graph showing NGF resulted in inhibition of HLA-G induced suppression of NK cell activity.

FIG. 4 is a bar graph showing NGF resulted in inhibition of TGF-beta induced suppression of NK cell activity.

FIG. 5 is a bar graph showing BDNF resulted in inhibition of IL-10 induced suppression of NK cell activity

FIG. 6 is a bar graph showing BDNF resulted in inhibition of VEGF induced suppression of NK cell activity.

FIG. 7 is a bar graph showing BDNF resulted in inhibition of HLA-G induced suppression of NK cell activity.

FIG. 8 is a bar graph showing BDNF resulted in inhibition of TGF-beta induced suppression of NK cell activity.

FIG. 9A is a bar graph showing suppression of tumor growth by administration of neural progenitor cells in mice injected with B16 melanoma cells.

FIG. 9B is a bar graph showing suppression of tumor growth by administration of neural progenitor cells in mice injected with 4T1 breast cancer cells.

FIG. 9C is a bar graph showing suppression of tumor growth by administration of neural progenitor cells in mice injected with LLC lung cancer cells.

FIG. 9D is a bar graph showing suppression of tumor growth by administration of neural progenitor cells in mice injected with C2-a glioma cells.

FIG. 9E is a bar graph showing suppression of tumor growth by administration of neural progenitor cells in mice injected with CD26+ cells.

### DETAILED DESCRIPTION OF THE INVENTION

The invention teaches the utilization of neurons and neuronal-like cells for altering the tumor microenvironment. In some embodiments the invention utilizes neurons or neuron-like cells differentiated from earlier progenitor cells. The generation of neurons from progenitor cells or from stem cells is known in the art [36-41]. Furthermore ability of said neuronal cells to integrate into host or alternative tissues has been demonstrated [42, 43]. In one embodiment of the invention administered pluripotent cells differentiate into the neuronal lineage and induce tumor regression or enhanced sensitivity of tumors to means known to induce tumor regression.

“Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells.

The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention.

Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma.

In the context of the present invention the term “culturing” refers to the in vitro propagation of cells or organisms in media of various kinds. It is understood that the descendants of a cell grown in culture may not be completely identical (morphologically, genetically, or phenotypically) to the parent cell. A suitable culturing medium can be selected by the person skilled in the art and examples of such media are RPMI medium or Eagles Minimal Essential Medium (EMEM).

The terms “vaccine”, “immunogen”, or immunogenic composition” are used herein to refer to a compound or composition that is capable of conferring a degree of specific immunity when administered to a human or animal subject. As used in this disclosure, a “cellular vaccine” or “cellular immunogen” refers to a composition comprising at least one cell population, which is optionally inactivated, as an active ingredient. The immunogens, and immunogenic compositions of this invention are active, which mean that they are capable of stimulating a specific immunological response (such as an anti-tumor antigen or anti-cancer cell response) mediated at least in part by the immune system of the host. The immunological response may comprise antibodies, immunoreactive cells (such as helper/inducer or cytotoxic cells), or any combination thereof, and is preferably directed towards an antigen that is present on a tumor towards which the treatment is directed. The response may be elicited or restimulated in a subject by administration of either single or multiple doses.

A compound or composition is “immunogenic” if it is capable of either: a) generating an immune response against an antigen (such as a tumor antigen) in a naive individual; or b) reconstituting, boosting, or maintaining an immune response in an individual beyond what would occur if the compound or composition was not administered. A composition is immunogenic if it is capable of attaining either of these criteria when administered in single or multiple doses.

The term “T-cell response” means the specific proliferation and activation of effector functions induced by a peptide in vitro or in vivo. For MHC class I restricted cytotoxic T cells, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, preferably granzymes or perforins induced by peptide, or degranulation.

The term “peptide” is used herein to designate a series of amino acid residues, connected one to the other typically by peptide bonds between the alpha-amino and carbonyl groups of the adjacent amino acids. The peptides are preferably 9 amino acids in length, but can be as short as 8 amino acids in length, and as long as 10, 11, 12, or even longer, and in case of MHC class II peptides (e.g. elongated variants of the peptides of the invention) they can be as long as 15, 16, 17, 18, 19, 20 or 23 or more amino acids in length.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoro-acetate) salts. It has to be noted that the salts of the peptides according to the present invention differ substantially from the peptides in their state(s) in vivo, as the peptides are not salts in vivo.

The term “peptide” shall also include “oligopeptide”. The term “oligopeptide” is used herein to designate a series of amino acid residues, connected one to the other typically by peptide bonds between the alpha-amino and carbonyl groups of the adjacent amino acids. The length of the oligopeptide is not critical to the invention, as long as the correct epitope or epitopes are maintained therein. The oligopeptides are typically less than about 30 amino acid residues in length, and greater than about 15 amino acids in length.

The term “polypeptide” designates a series of amino acid residues, connected one to the other typically by peptide bonds between the alpha-amino and carbonyl groups of the adjacent amino acids. The length of the polypeptide is not critical to the invention as long as the correct epitopes are maintained. In contrast to the terms peptide or oligopeptide, the term polypeptide is meant to refer to molecules containing more than about 30 amino acid residues.

A peptide, oligopeptide, protein or polynucleotide coding for such a molecule is “immunogenic” (and thus is an “immunogen” within the present invention), if it is capable of inducing an immune response. In the case of the present invention, immunogenicity is more specifically defined as the ability to induce a T-cell response. Thus, an “immunogen” would be a molecule that is capable of inducing an immune response, and in the case of the present invention, a molecule capable of inducing a T-cell response. In another aspect, the immunogen can be the peptide, the complex of the peptide with MHC, oligopeptide, and/or protein that is used to raise specific antibodies or TCRs against it.

Cancer-testis antigens: The first TAAs ever identified that can be recognized by T cells belong to this class, which was originally called cancer-testis (CT) antigens because of the expression of its members in histologically different human tumors and, among normal tissues, only in spermatocytes/spermatogonia of testis and, occasion-ally, in placenta. Since the cells of testis do not express class I and II HLA molecules, these antigens cannot be recognized by T cells in normal tissues and can therefore be considered as immunologically tumor-specific. Well-known examples for CT antigens are the MAGE family members and NY-ESO-1. Differentiation antigens: These TAAs are shared between tumors and the normal tissue from which the tumor arose. Most of the known differentiation antigens are found in melanomas and normal melanocytes. Many of these melanocyte lineage-related proteins are involved in biosynthesis of melanin and are therefore not tumor specific but nevertheless are widely used for cancer immunotherapy. Examples include, but are not limited to, tyrosinase and Melan-A/MART-1 for melanoma or PSA for prostate cancer. Over-expressed TAAs: Genes encoding widely expressed TAAs have been detected in histologically different types of tumors as well as in many normal tissues, generally with lower expression levels. It is possible that many of the epitopes processed and potentially presented by normal tissues are below the threshold level for T-cell recognition, while their over-expression in tumor cells can trigger an anticancer response by breaking previously established tolerance. Prominent examples for this class of TAAs are Her-2/neu, survivin, telomerase, or WT1. Tumor-specific antigens: These unique TAAs arise from mutations of normal genes (such as .beta.-catenin, CDK4, etc.). Some of these molecular changes are associated with neoplastic transformation and/or progression. Tumor-specific antigens are generally able to induce strong immune responses without bearing the risk for autoimmune reactions against normal tissues. On the other hand, these TAAs are in most cases only relevant to the exact tumor on which they were identified and are usually not shared between many individual tumors. Tumor-specificity (or -association) of a peptide may also arise if the peptide originates from a tumor- (-associated) exon in case of proteins with tumor-specific (-associated) isoforms. TAAs arising from abnormal post-translational modifications: Such TAAs may arise from proteins which are neither specific nor overexpressed in tumors but nevertheless become tumor associated by posttranslational processes primarily active in tumors. Examples for this class arise from altered glycosylation patterns leading to novel epitopes in tumors as for MUC1 or events like protein splicing during degradation which may or may not be tumor specific. Oncoviral proteins: These TAAs are viral proteins that may play a critical role in the oncogenic process and, because they are foreign (not of human origin), they can evoke a T-cell response. Examples of such proteins are the human papilloma type 16 virus proteins, E6 and E7, which are expressed in cervical carcinoma. T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

Therefore, TAAs are a starting point for the development of a T cell based therapy including but not limited to tumor vaccines. The methods for identifying and characterizing the TAAs are usually based on the use of T-cells that can be isolated from patients or healthy subjects, or they are based on the generation of differential transcription profiles or differential peptide expression patterns between tumors and normal tissues. However, the identification of genes over-expressed in tumor tissues or human tumor cell lines, or selectively expressed in such tissues or cell lines, does not provide precise information as to the use of the antigens being transcribed from these genes in an immune therapy. This is because only an individual subpopulation of epitopes of these antigens are suitable for such an application since a T cell with a corresponding TCR has to be present and the immunological tolerance for this particular epitope needs to be absent or minimal. In a very preferred embodiment of the invention it is therefore important to select only those over- or selectively presented peptides against which a functional and/or a proliferating T cell can be found. Such a functional T cell is defined as a T cell, which upon stimulation with a specific antigen can be clonally expanded and is able to execute effector functions (“effector T cell”).

Differentiation from Embryonic Stem Cells

In some embodiments of the invention pluripotent stem cells such as embryonic stem cells are differentiated into neuronal lineage cells. Numerous differentiation protocols are known in the art, some embodiments include treatment with nerve growth factor (NGF) [44, 45], HGF-1 [46], GDNF [47, 48], BDNF [49], VEGF [50], FGF-2 [51], the MK gene product [52], retinoic acid and oxidative stress [53-58], interleukin-1beta, glial cell line-derived neurotrophic factor, neurturin, transforming growth factor-beta(3) and dibutyryl-cyclic AMP [59], DMSO [60], leukemia inhibitory factor (LIF) [61, 62], brn-2 transfection [63], PiTX3 transfection [64], CREG [65], HuD transfection [66], LUZP transfection [67], astrocyte conditioned media [68-70], contact with glial cells or conditioned media [71, 72], dorsal root ganglia conditioned media [73], neural stem cell conditioned media [74], coculture with Sertoli cells [75], staurosporine [76], VIP and PACAP [77-79], 17-beta estradiol [80], ascorbic acid [81], neurotrophin [82], noggin [83-85], adherent conditions [86], nurr1 [87], methotrexate [88], notch [89], vitamin b12 and heparin [90], mu and kappa opioids [91], cyclopamine [92], selegiline [93], electrical stimulation [94],

In other embodiments differentiation of embryonic stem cells into neuronal cells is accomplished by blockade of specific molecular pathways. Pathways whose blockade or inhibition is associated with neuronal differentiation including the BMP pathway [95],

Specific neuronal differentiation protocols are known in the art and useful for specific cancer differentiation, modulation protocols covered by the potent. For example, in some embodiments dopaminergic neurons are needed. Protocols for generation of said dopaminergic neurons are known in the art [77, 96-114].

In other embodiments certain proteins of motor neurons are harness for anticancer/immunomodulator effects. Differentiation in motor neurons is described and incorporated by reference [115-121].

In some embodiments of the invention, serotonin producing neurons are needed for implantation in the tumor or peritumor sites. Generation of serotonin neurons is described in the following and incorporated by reference [122].

In other embodiments the invention teaches administration of cholinergic neurons. The generation of cholinergic neurons is incorporated by reference [123].

In other embodiments the invention teaches for administration of sympathetic neurons. Generation of sympathetic neurons is described and incorporated by reference [124].

Specifically in one embodiment generation of serotonin neurons is achieved as follows: embryoid bodies (EBs), are transferred to serum-free medium with 1% insulin-transferrin-selenium for 7 days to induce neural precursor cell (NPC) formation. NPCs are cultured in medium with 1% N-2 neural supplement and human fibroblast growth factor 2 (FGF2, 10 ng/ml) for 7 days to stimulate cell proliferation. Lastly, NPCs are dispersed into single cells and cultured without FGF2 for another 7 days to obtain terminal differentiation. Terminal cells are characterized for neuronal and serotonergic markers. Over 95% of the NPCs were immunopositive for nestin and Musashi1. Terminally differentiated cells appear in both small and large morphologies. Most (>95%) of the mature cells (both small and large) are immunopositive for neuron-specific nuclear protein (NeuN), synaptophysin, microtubule-associated protein (MAP2C), Tau-1, neurofilament 160 (NF-160), beta-tubulin (TujIII), tryptophan hydroxylase (TPH), serotonin, the serotonin reuptake transporter (SERT), estrogen receptor-beta (ERbeta), and progestin receptor (PR), but not estrogen receptor-alpha (ERalpha). Less than 2-3% of cells are positive for tyrosine hydroxylase (TH).

### Neuronal Markers

In some embodiments of the invention, selection of differentiated, or semi-differentiated cells is achieved through specifically isolating cells expressing neuronal markers [125-128]. Said markers include GAP-43 and NF-165 [129], Nurr1 and tyrosine hydroxylase (TH) [59], nestin [130], voltage-dependent (K+, Na+, Ca2+) and receptor-operated (GABAA, glycine, AMPA, NMDA receptors) ionic channels [131], GAD 1 and GAD67 [132], IZP6 [133], ABCA2 [134].

In some embodiments of the invention, neuronal or neuronal-like cells are differentiated in a manner to promote synaptic connections between neurons prior to or subsequent to intratumor administration [135].

In some embodiments of the invention, administration of neuronal cells into tumors is accomplished with the goal of inducing immunity to the tumor endothelium. Immunological targeting of tumor endothelium is appealing based on: a) For every tumor endothelial cell therapeutically neutralized approximately 200-300 tumor cells perish, thus reducing ability of tumors to lose expression of antigens; b) The immune system is in direct contact with the tumor endothelium, while immune access inside tumors is difficult due to areas of necrosis and high interstitial pressure; and c) Demonstrated prior efficacy of other anti-angiogenesis inhibitory compounds such as bevacizumab [136, 137]. Furthermore, the elevated expression of Fas Ligand on the tumor endothelium mediates the selective killing of CD8+ Tumor Infiltrating Lymphocytes (TIL) allowing for a predominance of FoxP3+T regulatory cells (Treg) to infiltrate the tumor microenvironment, demonstrating that the tumor blood vessels act as an immunological barrier promoting tumor tolerance [138]. Immune mediated destruction of the tumor endothelium has been shown to significantly increase TILs in mouse models, correlated with induction of anti-tumor immunity [139]. Another further potential benefit of targeting the tumor associated vasculature is the potential of sensitizing tumors to radiotherapy [140], in part due to the selective thrombotic and apoptotic effects irradiation has on the tumor vasculature [141-144]. Current tyrosine kinase inhibitors blocking angiogenesis systemically inhibit pro-angiogenic factors such as Vascular Endothelial Growth Factor (VEGF) or Angiopoetin 1-2, slowing blood vessel formation without differentiating between tumor and healthy angiogenesis. However, therapeutics that stimulate direct damage to the tumor endothelium have been shown to activate the coagulation cascade, effectively cutting off blood supply to the tumor and creating a hypoxic microenvironment conducive to necrosis and tumor regression [145]. A more effective anti-angiogenesis approach may be to stimulate the direct killing of the tumor endothelium through therapeutic vaccination. A fundamental question determining feasibility of vaccine-induced killing of tumor vasculature is whether antigens exist on the tumor endothelium that are not expressed on physiologically normal blood vessels, and whether immunity could be raised against such antigens. The roundabout receptor (ROBO)-4 is a transmembrane protein that was originally found to orchestrate the neuronal guidance mechanism of the nervous system [146]. ROB04 was found to be selectively expressed on tumor endothelial cells but not healthy vasculature [147]. Zhuang et al demonstrated that mice immunized with the extracellular domain of mouse Robo4, showed a strong antibody response to Robo4, with no objectively detectable adverse effects on health, including normal menstruation and wound healing. Robo4 vaccinated mice showed impaired fibrovascular invasion and angiogenesis in a rodent sponge implantation assay, as well as a reduced growth of implanted syngeneic Lewis lung carcinoma. The anti-tumor effect of Robo4 vaccination was present in CD8 deficient mice but absent in B cell or IgG1 knockout mice, suggesting antibody dependent cell mediated cytotoxicity as the anti-vascular/anti-tumor mechanism [148]. Another antigen that is more ubiquitously found throughout the body, but with higher expression on tumor endothelial cells is the VEGF receptor 2 (VEGFR2) which is typically found on hematopoietic stem cells and endothelial progenitor cells [149-154]. Despite expression on non-malignant tissue, successful induction of antitumor immunity has been demonstrated using various immunization means against this antigen. Yan et al utilized irradiated AdVEGFR2-infected cell vaccine-based immunotherapy in the weakly immunogenic and highly metastatic 4T1 murine mammary cancer model. Lethally irradiated, virus-infected 4T1 cells were used as vaccines. Vaccination with lethally irradiated AdVEGFR2-infected 4T1 cells inhibited subsequent tumor growth and pulmonary metastasis compared with challenge inoculations. Angiogenesis was inhibited, and the number of CD8+T lymphocytes was increased within the tumors. Antitumor activity was also caused by the adoptive transfer of isolated spleen lymphocytes, thus demonstrating induction of tumor specific immunity [155]. Other approaches have been utilized to induce immunity to VEGFR2, which resulted in induction of tumor regression without systemic toxicities [156-161]. Other approaches have been utilized to induce immunity to VEGFR2, which resulted in induction of tumor regression without systemic toxicities [156-161]. Tumor endothelial marker 1 or endosialin is another antigen found selectively on the tumor vasculature. Facciponte et al demonstrated that a DNA vaccination targeting endosialin reduced tumor vascularity, increased CD3+ T cell infiltration, and was correlated with significant inhibition of tumor growth. Epitope spreading to tumor antigens following the initial immune response against the tumor vasculature gives evidence that targeting the tumor endothelium may activate a cascade of pathways conducive to tumor regression. Additionally, the DNA vaccination against endosialin did not affect other angiogenesis dependent physiological processes, exhibiting no adverse effects on menstruation, embryonic development, pregnancy, and wound healing in mouse models [139]. Other markers associated with tumor blood vessels have been utilized therapeutically in animal models for vaccination purposes including survivin [162-164], endosialin [165], and xenogeneic FGF2R [166], VEGF [167], VEGF-R2 [168], MMP-2 [169], and endoglin [170, 171]. Although the genomic instability of tumor endothelial cells is less than that of tumor endothelial cells, thus reducing the possibility of immune mediated antigen loss, some mutational activity has been reported in tumor associated vascular cells [172, 173]. Accordingly, a polyvalent vaccine approach targeting the immune system towards a plethora of endothelial cells antigens specific to the tumor endothelium may be more effective. With this approach comes a heightened theoretical risk of autoimmunity. Despite these theoretical concerns successful immunization against tumor endothelium has been performed utilizing proliferating Human Umbilical Vein Endothelial Cells (HUVEC). Wei et al demonstrated that vaccination of mice fixed xenogeneic whole endothelial cells (in the form of HUVEC) as a vaccine was effective in affording protection from tumor growth, inducing regression of established tumors and prolonging survival of tumor-bearing mice. Additionally, the authors found that, immunity targeted to tumor vasculature was induced and was responsible for the anti-tumor activity, which was not associated with toxicity towards non-malignant tissues [174, 175]. Additionally, in a 17 patient clinical trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities [176]. The clinical efficacy of using HUVEC vaccination to break tolerance to tumor angiogenesis has also been demonstrated in patients with colorectal cancer and malignant brain tumors without any observable adverse effects on healthy angiogenesis [145].

In some embodiments of the invention, utilization of pluripotent stem cells obtained from spermatogonia precursors are used as a source to create neurogenic cells [177].

In some embodiments of the invention, neurogenic cells are grown in a hollow fiber culture means and conditioned media and/or exosomes and/or microvesicles are collected and administered into the tumor alone or in combination with therapeutic means. Methods of utilizing hollow fiber culture techniques are described and incorporated by reference [178].

Angiogenesis, the outgrowth of new blood vessels from pre-existing capillaries and post-capillary venules, occurs during embryonic development, in the uterus during the menstrual cycle, in the process of wound healing, and in pathological conditions [179]. In healthy adults, endothelial cells can maintain a quiescent state for years, whereas they proliferate and migrate to form new vessels in response to inflammatory conditions and during tumor growth. Several studies have estimated that tumor associated endothelial cells proliferate 30-40 times faster relative to endothelial cells found in healthy vasculature [180-182]. Based on estimates that tumors fail to grow beyond 1-2 mm in the absence of new capillary growth, Dr. Judah Folkman put forth the central hypothesis that tumors release diffusible factors that stimulate endothelial cell proliferation in host capillary blood vessels [183]. Indeed, it has been estimated that eradication of one endothelial cell is capable of neutralizing of up to 100-300 tumor cells [184]. Since the immune system is in direct contact with the tumor vasculature, vaccination against tumor endothelium is theoretically very promising for breaching the immunological barriers created by the tumor microenvironment.

The goal in vaccination strategies is to raise immunity against antigens present in tumor endothelium while avoiding antigens that cross-react with healthy vasculature, thereby preventing deleterious autoimmune reactions. Since the landmark publication by Dr. Folkman, a catalog of molecular players involved in the process of tumor angiogenesis have been identified and characterized. However, clinical outcomes of traditional anti-angiogenic therapies such as monoclonal antibodies have improved patient survival rates only modestly [185]. Vaccination against endothelial cells is poised to overcome the existing problems of drug resistance and adverse side effects associated with other approaches. This report reviews vaccination strategies against the tumor endothelium that have been tested to date, including DNA, protein and peptide vaccines using tumor-endothelium-associated antigens, as well as polyvalent vaccines comprised of whole endothelial cells. Very encouraging data point toward the efficacy of vaccination in raising humoral and cell-mediated immunity against angiogenesis-associated antigens in cancer. In this discussion, we also highlight our novel approach wherein placenta-derived endothelial cell lysates (ValloVax™) are used as a source of antigen for vaccinating against proliferating tumor endothelial cells in tumors.

Endothelium is a dynamic and heterogeneous structure influenced by environmental factors such as shear stress, oxygen content of the blood, chemokines, cytokines, and changes in the content of the extracellular matrix [186]. Whereas resting endothelium serves to maintain blood fluidity, regulate blood flow, control vessel wall permeability, and quiesce circulating lymphocytes [187], environmental cues cause endothelial cells to become activated to proliferate, migrate, and form new branches (sprouting). In the tumor milieu, aberrantly elevated and chronic production of angiogenic factors leads to endothelial activation, vascular irregularities, and immune suppression, which are among the well-recognized hallmarks of cancer proliferation [188]. Whereas the structure of normal vascular endothelium is hierarchical and organized, the activated endothelium in cancer consists of dilated and tortuous blood vessels that are chaotically interconnected, leading to heterogeneous vessel density within the tumor, erratic blood flow, and focal regions of hypoxia [189, 190]. At the cellular level, tumor endothelial cells display a disorganized morphology, being loosely connected and exhibiting increased vascular permeability [191]. These conditions cause impaired oxygen and nutrient delivery, conditions which in turn trigger angiogenesis, thereby further promoting tumor endothelial cell activation and vascular growth to meet the metabolic demands of the tumor. In this manner, cancer cells and endothelial cells are involved in a positive feedback loop stimulating each other's growth. At the same time, the vascular malformations in the tumor milieu serve as obstacles to effective penetration of anti-tumor lymphocytes and chemotherapy drugs into the tumor mass.

Although tumor endothelial cells are considered to be genetically stable as compared to tumor cells, angiogenic factors can fuel genetic aberrations of tumor endothelium. Indeed, resistance of tumor endothelial cells in diverse cancers has been described; for example, tumor endothelial cells in renal carcinoma are reportedly resistant to serum starvation [1192], breast tumor endothelial cells are resistant to vincristine-induced apoptosis [193] and hepatocellular carcinoma endothelial cells are resistant to 5-fluorouracil and doxorubicin [194]. One study demonstrated aneuploid chromosomes and abnormal centrosomes in tumor endothelial cells [195]. Signaling in response to the dysregulated over-production of angiogenic factors, including VEGF and FGF, in absence of endothelial stability promoting factors such as PDGF-BB was found to contribute to aneuploidy and centrosome duplication in tumor endothelial cells [196]. Moreover, tumor endothelial cells with excess centrosomes exhibit apoptosis resistance and formation of aberrant spindle projections, likely attributable to gain or loss of chromosome encoding genes involved in proliferation, survival, and adhesion [196]. At the mechanistic level, VEGF secreted from tumors affects tumor endothelial cells by upregulating MDR1, a gene encoding P-gp, which is a transmembrane glycoprotein that is as a multidrug transporter that potentiates resistance to several anti-cancer agents [197]. For example, paclitaxel, which is used to treat several types of cancer together with anti-angiogenic drugs, is transported by P-gp. The VEGFR kinase inhibitor, Ki8751, and a phosphatidylinositol 3-kinase-Akt inhibitor, LY294002, effectively block tumor-induced MDR1 up-regulation, suggesting that VEGF in the tumor microenvironment is an underlying factor in acquired drug resistance [197]. Vaccination against angiogenesis-associated antigens therefore could serve as a valuable asset for improving the efficacy of other cancer therapeutics.

Heterogeneity among tumor endothelial cells, as choreographed by the over-activity of certain angiogenic pathways, also shields tumors by affecting their visibility to the immune system. Production of angiogenic molecules by tumors inhibits the expression of adhesion molecules involved in leukocyte interactions with blood vessel walls, including intercellular adhesion molecule-1 (ICAM-1), vascular cell adhesion molecule-1 (VCAM-1), E-selectin and CD34 [198]. These features of tumor endothelium prevent adhesion and extravasation of effector T cells into the tumor. Additionally, tumor endothelial cells have been described as anergic, marked by unresponsiveness to inflammatory cytokines that would normally induce adhesion molecule expression but instead allow the endothelium to escape immune surveillance [198, 199]. The anergic phenotype of tumor endothelial cells can be reversed by anti-angiogenesis therapy, which upregulates the expression of endothelial adhesion molecules in the tumor vasculature [200, 201]. The tumor endothelium is also prohibitive to entry of tumor-reactive T lymphocytes by suppression or direct killing of effector T cells via molecules such as Fas ligand (FasL) [138]. Notably, VEGF-A, interleukin 10 (IL-10) and prostaglandin E2 (PGE2) are involved in eliciting FasL expression in endothelial cells, as evidenced by observations that pharmacological inhibition of these molecules leads to a marked influx of tumor-rejecting CD8+ cells and tumor growth suppression in mice. Hence, anti-angiogenesis vaccination can address the immunological firewall that the tumor endothelium imposes to protect the cancer.

Cancer vaccines consist of tumor-associated antigens delivered in a pro-inflammatory context to generate potent antitumor immune responses that overcome the cancer's varied immunosuppressive mechanisms. Vaccination strategies directed against tumor-associated endothelium are designed to take advantage of both quantitative and qualitative differences between tumor endothelial cells and non-malignant endothelial cells. Ideal vaccine candidates include receptors/proteins that are upregulated in tumor endothelium, owing to its activated and proliferating state, but are sparsely expressed during physiological angiogenesis in healthy adult tissues. Notably, angiogenesis-associated molecules can be over-expressed on endothelial cells in an organ- or tumor-specific manner, and can sometimes be expressed on resting endothelial cells [202]. Thus, an attractive tumor endothelial vaccine should be capable of eliciting anti-angiogenesis and anti-cancer immunity against diverse tumor types, while avoiding autoimmune reactions against physiological angiogenesis, such that occur in the female reproductive tract and during wound healing, or against quiescent endothelium.

Whether immunity and/or autoimmunity ensue as a result of vaccination is dependent not only on the target tumor endothelial antigen but also on the vaccination protocol. Candidate vaccines have employed numerous delivery systems/vectors, including proteins, peptides, dendritic cells pulsed with the antigen(s), naked DNA or recombinant DNA delivered by carriers, and mRNA vaccines, as well as whole cell vaccines or endothelial membrane components. To develop rationales for clinical vaccine design, pre-clinical studies have been conducted for many angiogenesis-associated molecules using various delivery systems. In some cases, differences in safety and efficacy between vaccines hinge on a plethora of secondary variables including the use of different delivery vectors, the choice of adjuvants and the varying routes of vaccine administration.

Two general approaches to anti-angiogenesis vaccination have yielded promising results for inducing specific and robust immunity against tumor endothelium and reducing tumor growth and metastasis: 1) Vaccines expressing defined angiogenesis-associated antigens; and, 2) Vaccines comprised of whole endothelial cells and/or mixtures of endothelial antigens. In the latter category are vaccines consisting of placenta-derived endothelial antigens, an approach currently being advanced by Batu Biologics, Inc. (ValloVax™ vaccine).

Evidence has shown the feasibility of targeting molecules that are expressed by angiogenic endothelium, and the data also suggest that anti-angiogenic vaccination can be applied synergistically with tumor immunotherapy and/or chemotherapy to invoke anti-tumor immunity. Theoretically, the approach of targeting defined angiogenesis-associated molecules on tumor endothelium has the risk to evoke only a transient decrease in cancer progression, owing to the fact that angiogenesis can proceed via compensatory pathways. Despite this caveat, success at achieving anti-angiogenic immunity has been demonstrated by vaccinating against the following antigens.

VEGF-A and VEGFR: VEGF/VEGFR is perhaps the best-studied angiogenic pathway involved in the growth and survival of tumor endothelium. Clinical therapies addressing tumor angiogenesis have therefore largely focused on inhibiting VEGF and its cognate receptor. VEGF-A exists in several pro-angiogenic variants that are secreted by both tumor cells and endothelial cells and activate VEGFR1 and VEGFR2. Numerous vectors have been administered for vaccinating against VEGF, including a Xenopus VEGF DNA vaccine that was protective and therapeutic in several tumor models in mice, an effect that was mediated by anti-tumor activity of CD4+ lymphocytes [167]. A phase I clinical study has investigated CIGB-247, a vaccine comprising a human VEGF variant molecule in combination with a bacterial adjuvant, in 30 patients with advanced solid tumors [203]. After eight consecutive weeks of subcutaneous immunization and re-vaccination on week 12, this vaccine was safe at three dose levels and also demonstrated immunogenicity in three sequential analyses of patients' blood samples. Despite the critical importance of VEGF in hematopoiesis [204-206], no abnormalities in leukocytes or megakaryocytes where found, and additionally, serum biochemistry parameters where not altered. Based on this clinical report, a vaccine based on VEGF is a highly promising cancer treatment strategy. The implications of this body of research are that immunity can be induced to self-antigens, without the fear of triggering a fulminant autoimmune response.

VEGFR2 (also FLK-1 and KDR), which is highly expressed on proliferating endothelial cells of the tumor vasculature, has also been the focus of numerous pre-clinical studies. Animal models using VEGFR2 DNA, protein, and peptide vaccines have demonstrated their ability to elicit potent humoral and cellular immunity, suppression of angiogenesis, tumor necrosis and/or suppression of metastastatic progression [157, 168, 207-212]. Interestingly, none of the aforementioned studies have reported hematopoietic or other abnormalities.

Since VEGFR2 is also expressed at lower levels in normal vascular endothelium, vaccination could theoretically cause side effects or an autoimmune response. Neithammer et al. reported that in murine models a DNA vaccine against VEGFR2 suppressed angiogenesis in the tumor vasculature as verified by in-vitro inhibition of endothelial cell proliferation, deposition of antibodies in tumor vasculature, a reduction of microvessel density and antitumor activity in vivo without impairment of fertility, neuromuscular performance or hematopoiesis; however, a slight delay in wound healing in immunized mice was noted [213]. In another report, there was no delay in wound healing reported in response to a dendritic cell-based VEGFR2 vaccine although reduced litter sizes and fetal loss were reported in vaccinated animals [214]. However, in other studies where VEGFR2 protein vaccines were found to generate potent immunity against tumor endothelium, no effects on wound healing, reproduction or other organ toxicities were observed [168, 215].

Immunogenic epitopes of VEGFR2 have been identified and peptide vaccines have been optimized for binding to MHC class I molecules in order to elicit activity of cytotoxic T lymphocytes (CTL) specific for tumor endothelium. In a clinical study of pancreatic cancer, administration of immunogenic VEGFR2-169 peptide vaccine given in combination with gemcitabine, administration of this vaccine was well tolerated with no severe adverse events and peptide-specific CTL were induced [216]. Other than injection site reactions, other peptide vaccines tested for targeting VEGFR were also deemed to have manageable toxicities [161, 217, 218].

bFGF and FGF-R: Basic fibroblast growth factor (bFGF or FGF-2) and its receptor (FGFR-1, CD331) have been targeted in pre-clinical mouse models to evaluate their anti-angiogenic and anti-tumor effects. FGFR-1 is highly expressed on angiogenic endothelium as well as on endothelial progenitor cells [219, 220]. In one such study, vaccination with xenogeneic FGFR-1 plasmid DNA inhibited tumor endothelial cell proliferation and produced anti-cancer immunity in three murine models [221]. Studies utilizing recombinant protein vaccines targeting FGFR-1 demonstrated significantly decreased tumor volume compared to controls, as well as decreased microvessel density without any observable overt toxicity [222]. Peptide bFGF vaccines combined with adjuvants also induced antigen-specific antibody and cell-mediated responses [223, 224]. Importantly, in a study where a liposome-based peptide bFGF vaccine was administered in murine cancer models, immunity against tumor endothelium was elicited while physiological angiogenesis was unperturbed, as evidenced by normal wound healing times and no impairments in the reproductive ability of vaccinated animals or in the viability or health of the offspring [166].

αvβ3: The expression of several cell adhesion molecules, most notable integrin alpha(v)beta(3), has been associated with tumor angiogenesis [225].

Integrin αvβ3 plays a role in several physiological processes including angiogenesis, pathological neovascularization, and tumor metastasis [225]. An αvβ3 ligand vaccine induced a humoral response associated with significant antitumor activity without recourse of adjuvant therapy. The immune response was driven by antibody-dependent cellular toxicity and complement-directed cytotoxicity to the tumor-associated endothelial cells [226]. The effectiveness of utilizing antibodies blocking αvβ3 as a method of inhibiting angiogenesis, has led to the clinical development of monoclonal anti-αvβ3 antibodies for the treatment of advanced solid tumors. MEDI-522, a second generation humanized anti-αvβ3, successfully completed a Phase I study in patients with a variety of metastatic solid tumors, and a maximum tolerated dose was not identified by traditional dose-limiting toxicities [227].

Angiomotin: Another target of anti-angiogenic vaccine strategies is the angiostain binding protein angiomotin, a membrane-associated pro-angiogenic protein present on endothelial cells in angiogenic tissues [228]. Holmgren et al. used DNA vaccination with a xenogeneic (human) angiomotin DNA construct to increase immunogenicity of the vaccine, and injected the DNA intramuscularly followed by electroporation [229]. This vaccine protected mice from tumor challenge and acted synergistically with a vaccine targeting the Her-2 oncogene in a transgenic breast cancer model system. No evidence for autoimmunity in normal vasculature of the retinas was noted at 16 or 70 weeks post-treatment. In another report, an angiomotin DNA vaccine was shown to alter blood vessel architecture to increase permeability and improve the efficacy of chemotherapy in an animal model [230].

Endoglin (CD105): This transmembrane glycoprotein is primarily expressed in proliferating endothelial cells and is upregulated by hypoxia; therefore, endoglin is strongly expressed in tumor endothelial cells [231]. The functional role for endoglin in hematopoiesis is also exemplified by CD105 knockout studies where embryonic lethality occurred as a result of impaired angiogenesis in the yolk sac and heart defects. CD105 expression on tumor vessels is a prognostic factor correlated with poor overall and disease-free survival, tumor recurrence, and metastasis of various cancers [232, 233]. CD105 vaccination approaches employing bacterial surface display of protein [234] and orally administered DNA vaccines [235] effectively targeted the vasculature and inhibited tumor growth in the absence of observable effects on healthy tissues.

Survivin: This is an intracellular tumor-associated antigen that is upregulated in cancers and tumor endothelial cells but is not expressed in healthy differentiated tissues. Survivin is a member of the inhibitor of apoptosis protein family, and serves to inhibit programmed cell death, promote cellular proliferation and enhance angiogenesis [236]. Survivin vaccines elicit apoptosis of tumor cells and its vasculature with variable efficacy; while intradermal electroporation was found to be very effective in animal models, naked DNA administration is less effective in the prophylactic or therapeutic settings [162, 237].

Robo4: Robo4 is a member of the Roundabout family of proteins and is a transmembrane cell adhesion molecule that is specifically expressed in endothelial cells and hematopoietic stem cells and progenitor cells [238]. Robo4 guides the formation of the vasculature by controlling endothelial cell migration and coordinating blood vessel sprouting [239]. Tonic Robo4 signaling in healthy tissues maintains vascular integrity by inhibiting VEGF-induced endothelial cell migration and vascular permeability [240]. Robo4 is pathologically over-expressed in the tumor endothelium [241], it promotes atypical vascular patterning that reduces blood-tumor barrier permeability, thereby likely impeding the access of chemotherapy drugs to tumors [242]. In one study, a protein vaccine against this target was developed, comprised of the extracellular domain of mouse Robo4, fused to the Fc domain of human immunoglobulin within an adjuvant. Vaccinated mice had a strong antibody response to Robo4, impaired fibrovascular invasion in vitro, and reduced growth of implanted Lewis lung carcinoma [243]. Further studies will be needed to determine how breaking tolerance to Robo4 affects physiological angiogenesis, where it appears to counteract angiogenic signaling [244].

Tie-2/Angiopoietin Receptor: Tie-2 is an endothelium-specific receptor tyrosine kinase that binds to angiopoietin and is upregulated on diseased endothelium in diverse conditions including cancer, atherosclerosis, and macular degeneration. A protein vaccine based on chicken Tie-2 reduced growth of hepatomas and melanomas in mice, whereas a murine Tie-2 vaccine was ineffective, indicating the need for further enhancement of this protein's immunogenicity [245]. The chicken Tie-2 vaccine exhibited apoptotic and anti-angiogenic effects on endothelial cells. The ligand for Tie-2, angiopoietin-2 is also over-expressed during neoangiogenesis in tumors and acts synergistically with VEGF in promoting tumor vascularization [246].

EGFR: This receptor can be activated by diverse ligands; namely, EGF, transforming growth factor-α (TGF-α), amphiregulin, betacellulin, heparin-binding EGF, or epiregulin that are locally secreted by cancer cells and can act in an autocrine fashion. EGFR signaling regulates the secretion of several different angiogenic growth factors by tumor cells including VEGF [247]. Co-expression of EGFR ligands and EGFR is associated with malignant tumor phenotypes and poor prognosis in cancer patients [248-250]. Monoclonal antibodies and vaccines targeting this pathway have been implemented clinically, including the monoclonal antibodies cetuximab, panitumumab and nimotuzumab; the small tyrosine kinase molecules erlotinib and gefitinib; the EGF-based cancer vaccine CIMAvax®; and a EGFR-based HER-1 cancer vaccine. CIMAvax-EGF is a therapeutic cancer vaccine of human recombinant epidermal growth factor (EGF) conjugated to a carrier protein from Neisseria meningitides. The vaccine induces antibodies against EGF and confers a survival advantage in patients with non-small-cell lung cancer [251]. Studies of post-operative wound healing revealed that there are no deleterious effects on physiological angiogenesis associated with targeting the EGF pathway [252].

HP59: This polypeptide is expressed as a marker for neonatal lung vasculature and adult pathological angiogenesis. HP59 protein has been identified in the vasculature of many types of tumors including lung, colon, ovary, and breast cancers of different stages, but not in the corresponding normal tissues; therefore, this protein could be an ideal target antigen for vaccinating against tumor endothelium [253]. Vaccination of mice with immunogenic HP59 peptides attenuated the growth of Lewis lung tumors and inhibited pathological angiogenesis, as evidenced by an absence of HP59-expressing blood vessels. HP59 possesses a 41-amino acid sequence at the NH₂ terminal that has no homology with any known protein; therefore, this antigen could be used for raising specific immunity against the pathological vasculature in human cancer [253].

PDGFR: This tyrosine kinase receptor is activated by members of the platelet-derived growth factor family, and is broadly involved in regulating cellular proliferation and differentiation in physiological and pathological contexts. PDGF-B, the ligand, produced by tumor cells binds to PDGFR on stromal and perivascular cells to promote tumor growth and angiogenesis, as well as eliciting other pathways of tumor nourishment [254, 255]. PDGFRβ supports angiogenesis in two distinct ways: 1) By activating pericytes, which support endothelial cell proliferation and elaboration of the tumor vasculature; and, 2) By upregulating expression of the proangiogeneic factor FGF-2 [255].

Anti-PDGF drugs have been widely used for treating various cancers; however, their mechanisms of action at a molecular level are not strictly anti-angiogeneic. Paradoxically, PDGF ligand inhibition can increase tumor angiogenesis, which supports increased tumor growth but, in doing so, normalizes blood vessel walls to allow for more efficient delivery of chemotherapy drugs [256, 257]. On this basis, inhibiting the PDGF pathway can serve as an effective adjunct to cytotoxic drugs and yield a net gain in eradicating tumors [256].

To assess the effects of vaccinating against PDGFRP, a DNA vaccine delivered via Salmonella typhimurium was given to mice orally in murine colon, breast and lung carcinoma model systems [258]. Significantly, in a therapeutic setting where tumors were implanted 20 days before vaccination, the size of tumors in the PDGFRβ-immunized group was one-fourth the size of the control group. Angiogenic markers within tumors were reduced and robust cytotoxic T cell responses against PDGFR-β were detectable, indicating that angiogenesis was effectively targeted.

TEM1: Tumor endothelial marker 1, also known as endosialin or CD248, is a type 1 membrane protein involved in developmental, physiological and pathological angiogenesis. TEM1 is overexpressed in the vasculature of carcinomas, brain tumors and sarcomas [259-262] and is present on endothelial cells, pericytes, and fibroblasts [263, 264]. A DNA vaccine expressing full-length mouse Tem1 cDNA fused to tetanus toxoid adjuvant inhibited tumor growth and progression without affecting reproduction or wound healing [139]. Splenocytes from TEM1 vaccinated mice secreted IFN-γ and lysed TEM1-expressing endothelial cells in vitro. These data support the use of TEM1 protein vaccine in conjunction with an adjuvant to break tolerance to tumor endothelium for the treatment of solid tumors

TEM8: Designing a tumor endothelial vaccine based on TEM8 is a very enticing possibility since this molecule is selectively upregulated on tumor endothelial cells and, unlike the majority of other putative vaccine targets, TEM8 is undetectable in endothelium during physiological angiogenesis [265]. The first TEM8 DNA vaccine was a syngeneic vaccine that was tested in a rat model of breast cancer and murine melanoma [266]. Although the vaccine had no activity as a single agent, TEM8 DNA significantly enhanced anti-tumor immunity when administered with a rat HER2 DNA vaccine for breast cancer, and also in combination with a tyrosine-related protein-1 DNA vaccine in melanoma. Ruan et al. developed a xenogeneic TEM8 DNA vaccine carried by xenogeneic Salmonella typhimurium that was capable of generating TEM8-specific CD8+ cytotoxic T cells and protected mice from lethal tumor challenge [267]. Another vaccination approach consisted of dendritic cells transduced with recombinant adenovirus encoding TEM8, which also effectively protected mice from lethal challenges against hepatocellular carcinoma [268]. Lastly, a DNA vaccine encoding syngeneic TEM8 and murine beta-defensin 2, which activates dendritic cells to stimulate potent immunity, was used to inhibit tumor growth in a murine colon cancer model [269]. This vaccine was also highly effective, causing the collapse of tumor vessels by evoking an antigen specific CD8+ T cell response. Further studies are warranted to determine whether TEM8-based vaccines can be safe and efficacious for clinical use.

In summary, several therapeutic approaches have been explored targeting single antigens expressed on the tumor endothelium, demonstrating the feasibility of targeting the tumor vasculature by vaccine therapy without inducing systemic autoimmunity to quiescent endothelium in vivo. Signals of efficacy have been observed in preclinical studies around these targets, and several tumor endothelium-targeting vaccines have made it into the clinical setting for the treatment of cancer. As such, in an effort to reduce the development of treatment resistance and to potentiate the strength of an immune response and resultant survival benefit, the authors would like to explore the concept of a polyvalent vaccine targeting several angiogenesis associated antigens.

### 2) Endothelial Cell Vaccines

Although great strides have been made in advancing anti-angiogenesis tumor vaccines against specific targets, it is clear that the presence of many interrelated and compensatory pathways, as well as genetic instability of tumor endothelium could theoretically overcome targeted inhibition in a clinical setting. Accordingly, polyvalent vaccination approaches are being developed using whole endothelial cells or isolated proteins from endothelial cell membranes. Another advantage of such polyvalent vaccines expressing numerous angiogenic antigens is to allow antigen-presenting cells to process and present immunodominant epitopes for generating anti-angiogenic immunity.

Preclinical Studies of Endothelial Cell Vaccines: Human umbilical vein endothelial cells (HUVEC) have been the standard for cell-based models of tumor angiogenesis, having the ability to proliferate extensively and expressing a number of pro-angiogenic molecules that mimic tumor neovasculature. Specifically, the aforementioned antigens such as VEGFR2, αvβ3 and endoglin, which are common biomarkers associated with tumor angiogenesis, are expressed in the primary culture of HUVECs [174]. In a report by Wei et al. [174], paraformaldehyde fixed xenogeneic whole HUVEC used as a vaccine markedly inhibited tumor growth in prophylactic and therapeutic murine cancer models. The anti-angiogenic effect of this vaccine depended on CD4+ T cells eliciting endothelial-specific antibody responses. Using a syngeneic vaccine consisting of fixed hepatic sinusosoidal endothelial cells, Okaji et al. also demonstrated potent preventative and therapeutic anti-tumor immunity in a lung metastasis model of murine colon cancer [270]. Both antibody and cytotoxic T cell responses against endothelial cells were detected in response to this vaccine. Similar studies performed in animal models have reported potent angiogenesis inhibition and tumor targeting when using syngeneic endothelial cell vaccines [271, 272], xenogeneic endothelial cells [273], and xenogeneic endothelial proteins [274]. Although the experimental design differences between these studies preclude any definitive conclusions concerning the optimal vaccine candidate, these data collectively provide a compelling proof of concept that tolerance to tumor endothelium can be broken using whole endothelial cells as vaccines.

Clinical Progress With Endothelial Cell Vaccines: HUVECs have been used in pilot studies to test the anti-angiogenic effects of vaccination in patients with malignant brain tumors and metastatic colorectal cancer [145, 176]. Vaccinations were performed using 5×10(7) HUVECs given intradermally on a weekly basis for the first month and the every two weeks from the second month onward. In a published report where a total of 230 vaccinations were administered to six patients with recurrent malignant brain tumors and three patients with metastatic colorectal cancer, MRI results showed partial or complete responses lasting for a minimum of nine months in three of the patients with brain tumors [145]. Moreover, antibodies directed against HUVEC antigens were detected in eight out of nine patients and HUVEC-specific CTLs were detected in six of seven tested patients. No adverse events were reported with the exception of skin reactions at the vaccine injection site.

Classical studies have shown that tumor infiltrating lymphocytes correlate with positive prognosis in various tumors [275-287]. The invention teaches means of increasing lymphocytic recognition of cancer endothelial cells. Unfortunately, there are several important factors that prevent efficacy of infiltrating lymphocytes. Firstly, tumor masses originate from tumor stem cells, which possess distinctly different antigenic composition [288-292]. Accordingly, infiltration of lymphocytes, while useful for targeting tumor stem cell progeny, may not actually reach, or recognize tumor stem cells. This is also relevant in light of studies showing tumor stem cells possess various immune evasive molecules such as DAF, IL-10 and HLA-G. Secondly, tumors are known to possess high interstitial pressure, which physically limits ability of lymphocytes to enter the tumor mass, which often possesses necrotic tissue. Thirdly, tumor acidosis, hypoxia, and high adenosine concentrations have been demonstrated to selectively inhibit cytotoxic cells and promote T regulatory cells.

In a related study, 17 patients with recurrent glioblastoma were treated with the HUVEC vaccine using the same protocol of intradermal delivery [176]. These patients had been previously treated with surgery and chemoradiotherapy, and were also undergoing salvage treatments including concomitant and adjuvant chemotherapy during the course of the study. The results showed that HUVEC vaccine therapy is feasible for recurrent glioblastoma based on significant prolongations of the tumor doubling times and reduced tumor growth rates at 3- and 6-month follow ups. Despite the fact that 352 vaccinations were performed, no adverse events were observed with the exception of skin reactions at the injection sites. For comparative purposes, the investigators point out that bevacizumab, a humanized monoclonal antibody to VEGF, is associated with grade 3 adverse events in 46.4% of patients when used alone and in 65.8% of patients when used in combination with chemotherapy [293], demonstrating that a HUVEC vaccine appears to be much safer than conventional anti-angiogenesis drug therapies [176]. The authors conclude that, for invasive and large tumors, HUVEC vaccination is feasible for use in combination with other treatment modalities and similar trials should be conducted for other types of cancer.

The invention teaches means of activating abscopal effect in cancer patients and enhancing such systemic effects through immunizing against tumor endothelium. In some embodiments abscopal effect is induced by irradiation in a manner to cause localized tumor cell death. One of the first described examples of abscopal effect was published in 1975 when systemic melanoma metastasis started regressing after localized radiation treatment [294]. Ohba et al reported the case of a 76 year old Japanese man with hepatocellular carcinoma that regressed after radiotherapy for thoracic vertebral bone metastasis. Serum levels of tumour necrosis factor-alpha increased after radiotherapy. The findings suggests that such abscopal related regression may be associated with host immune response, involving cytokines such as tumour necrosis factor-alpha [295]. Another case was reported of a 69-year-old woman with advanced uterine cervical carcinoma with toruliform para-aortic lymph node metastases that showed an abscopal effect of radiation therapy (effect out of irradiated field). The patient was admitted to our University Hospital in March 2005, and treated with radiation therapy only for the primary pelvic lesions without chemotherapy because of her severe economic status. After the treatment, not only did the cervical tumor in the irradiated field disappear, but the toruliform para-aortic lymph node swelling outside the irradiated field also spontaneously disappeared. The patient is still alive and well without relapse [296]. Okuma et al reported on a 63-year-old Japanese man underwent extended right hepatic lobectomy for hepatocellular carcinoma. During his follow-up examination, a single lung metastasis and a single mediastinal lymph node metastasis were found. Trans-catheter arterial embolization was initially attempted to treat the mediastinal tumor, however this approach failed to take effect and carried risks of spinal artery embolism. External-beam irradiation, with a dose of 2.25 Gy per fraction, was performed using an antero-posterior parallel-opposed technique (total dose, 60.75 Gy). A computed tomography scan performed one month after starting radiotherapy showed a remarkable reduction of the mediastinal lymph node metastasis. In addition to this, they observed spontaneous shrinking of the lung metastasis, which was located in the right lower lobe and out of the radiation field. No chemotherapy was given during the period. There has been no recurrence of either the lung metastasis or the mediastinal lymph node metastasis during a follow-up 10 years after the radiotherapy [297]. In another report an 80-year-old male with squamous cell carcinoma with bilobar hepatic metastases who underwent targeted Yttrium-90 radioembolization of the right hepatic lobe lesion. Subsequently, there was complete regression of the nontargeted, left hepatic lobe lesion [298].

Cases of abscopal effect have also been observed in chronic lymphocytic leukemia [199, 300], Merkel Cell Carcinoma [301], melanoma [302-304], renal cell carcinoma [305], myeloma [306], pancreatic cancer [307, 308], breast cancer [309, 310], renal cell carcinoma [311], diffuse Giant tumor [312], and non-small cell lung cancer [313].

In a murine study, mice bearing a syngeneic mammary carcinoma, 67NR, in both flanks were treated with Flt3-L daily for 10 days after local radiation therapy (RT) to only 1 of the 2 tumors at a single dose of 2 or 6 Gy. The second nonirradiated tumor was used as indicator of the abscopal effect. Data were analyzed using repeated measures regression. Radiation therapy (RT) alone led to growth delay exclusively of the irradiated 67NR tumor, as expected. Surprisingly, growth of the nonirradiated tumor was also impaired by the combination of RT and Flt3-L. As control, Flt3-L had no effect without RT. Importantly, the abscopal effect was shown to be tumor specific, because growth of a nonirradiated A20 lymphoma in the same mice containing a treated 67NR tumor was not affected. Moreover, no growth delay of nonirradiated 67NR tumors was observed when T cell deficient (nude) mice were treated with RT plus Flt3-L. The authors concluded that the results of their experiments demonstrated that the abscopal effect is in part immune mediated and that T cells are required to mediate distant tumor inhibition induced by radiation [314]. Van der Meerans et al. reported on an investigation of the radiation-induced inflammatory response in C57BL6/J mice after total abdominal or total-body irradiation at a dose of 15 Gy. The goal was to determine the radiation-induced inflammatory response of the gut and to study the consequences of abdominal irradiation for the intestine and for the lungs as a distant organ. A comparison with total-body irradiation was used to take into account the hematopoietic response in the inflammatory process. For both irradiation regimens, systemic and intestinal responses were evaluated. A systemic inflammatory reaction was found after abdominal and total-body irradiation, concomitant with increased cytokine and chemokine production in the jejunum of irradiated mice. In the lungs, the radiation-induced changes in the production of cytokines and chemokines and in the expression of adhesion molecules after both abdominal and total-body irradiation indicate a possible abscopal effect of radiation in our model. The effects observed in the lungs after irradiation of the abdomino-pelvic region may be caused by circulating inflammatory mediators consequent to the gut inflammatory response [315].

The invention teaches augmentation of abscopal effect by immunization with proteins and/or peptides and/or other antigenic materials that induce immunity to the tumor endothelial cells. Other means of stimulating immunity to endogenous tumor antigens have been described in the art to augment abscopal effect. In one experiment tumors were implanted s.c. in the right or both flanks. After local irradiation at the right flank, ECI301, a human macrophage inflammatory protein-1alpha variant was injected i.v. Tumor volumes were measured every 3 days after treatment. In Colon26 adenocarcinoma-bearing BALB/c mice, repeated daily administration (over 3-5 consecutive days) of 2 mug per mouse ECI301 after local irradiation of 6 Gy prolonged survival without significant toxicity, and in about half of the treated mice, the tumor was completely eradicated. Three weekly administrations of ECI301 after local irradiation also led to significant, although less effective, antitumor radiation efficacy. ECI301 also inhibited growth of other syngenic tumor grafts, including MethA fibrosarcoma (BALB/c) and Lewis lung carcinoma (C57BL/6). Importantly, tumor growth at the nonirradiated site was inhibited, indicating that ECI301 potentiated the abscopal effect of radiation. This abscopal effect observed in BALB/c and C57BL/6 mice was tumor-type independent. Leukocyte depletion studies suggest that CD8+ and CD4+ lymphocytes and NK1.1 cells were involved. Marked inhibition of tumor growth at the irradiated site, with complete tumor eradication and consistent induction of the abscopal effect, was potentiated by i.v. administration of ECI301. The results of this study may offer a new concept for cancer therapy, namely chemokine administration after local irradiation, leading to development of novel therapeutics for the treatment of advanced metastatic cancer [316]. Another study aiming to augment the abscopal effect examined CTLA-4 blockade as a means of immune stimulation.

TSA mouse breast carcinoma cells were injected s.c. into syngeneic mice at two separate sites, defined as a “primary” site that was irradiated and a “secondary” site outside the radiotherapy field. When both tumors were palpable, mice were randomly assigned to eight groups receiving no radiotherapy or three distinct regimens of radiotherapy (20 Gy×1, 8 Gy×3, or 6 Gy×5 fractions in consecutive days) in combination or not with 9H10 monoclonal antibody against CTLA-4. Mice were followed for tumor growth/regression. Similar experiments were conducted in the MCA38 mouse colon carcinoma model. In either of the two models tested, treatment with 9H10 alone had no detectable effect. Each of the radiotherapy regimens caused comparable growth delay of the primary tumors but had no effect on the secondary tumors outside the radiation field. Conversely, the combination of 9H10 and either fractionated radiotherapy regimens achieved enhanced tumor response at the primary site (P<0.0001). Moreover, an abscopal effect, defined as a significant growth inhibition of the tumor outside the field, occurred only in mice treated with the combination of 9H10 and fractionated radiotherapy (P<0.01). The frequency of CD8+ T cells showing tumor-specific IFN-gamma production was proportional to the inhibition of the secondary tumor. The authors concluded that fractionated but not single-dose radiotherapy induces an abscopal effect when in combination with anti-CTLA-4 antibody in two preclinical carcinoma models [317].

Similar results where observed when researchers attempted to improve the local and abscopal effect by modulating T cell immunity with systemic blockade of CTLA-4 signal. The growth of primary tumors was significantly inhibited by LRT while CTLA-4 antibody enhanced the antitumor effect. Growth delay of the second tumors was achieved when the primary tumor was radiated. LRT resulted in more T cell infiltration into both tumors, including Treg and cytotoxic T cells. Interestingly, the proportion of Treg over effector T cells in both tumors was reversed after CTLA-4 blockade, while CD8 T cells were further activated. The expression of the immune-related genes was upregulated and cytokine production was significantly increased. LRT resulted in an increase of TIT, while CTLA-4 blockade led to significant reduction of Tregs and increase of cytotoxic T cells in both tumors. The abscopal effect is enhanced by targeting the immune checkpoints through modulation of T cell immune response in murine mesothelioma [318].

Accordingly, in one embodiment of the invention addition of inhibitors to CTLA-4 such as antibodies, microbodies, siRNA, shRNA, antisense, shark or cameloid antibodies, or small molecules may be used together with tumor endothelial or tumor immunization means in order to augment abscopal effect of radiotherapy. Various concepts and regimens for utilization of CTLA-4 are known in the literature [319-324].

Use of another checkpoint inhibitor was reported to augment the abscopal effect. Researchers investigated the influence of PD-1 expression on the systemic antitumor response (abscopal effect) induced by stereotactic ablative radiotherapy (SABR) in preclinical melanoma and renal cell carcinoma models. We compared the SABR-induced antitumor response in PD-1-expressing wild-type (WT) and PD-1-deficient knockout (KO) mice and found that PD-1 expression compromises the survival of tumor-bearing mice treated with SABR. None of the PD-1 WT mice survived beyond 25 days, whereas 20% of the PD-1 KO mice survived beyond 40 days. Similarly, PD-1-blocking antibody in WT mice was able to recapitulate SABR-induced antitumor responses observed in PD-1 KO mice and led to increased survival. The combination of SABR plus PD-1 blockade induced near complete regression of the irradiated primary tumor (synergistic effect), as opposed to SABR alone or SABR plus control antibody. The combination of SABR plus PD-1 blockade therapy elicited a 66% reduction in size of nonirradiated, secondary tumors outside the SABR radiation field (abscopal effect). The observed abscopal effect was tumor specific and was not dependent on tumor histology or host genetic background. The CD11a(high) CD8(+) T-cell phenotype identifies a tumor-reactive population, which was associated in frequency and function with a SABR-induced antitumor immune response in PD-1 KO mice. It was concluded that SABR induces an abscopal tumor-specific immune response in both the irradiated and nonirradiated tumors, which is potentiated by PD-1 blockade [325].

While the classical immune checkpoints are known to be inhibitory cell surface molecules such as CTLA-4, PD-1, TIM-3, and LAG-3, cytokines can also inhibit abscopal effect. Accordingly in one embodiment of the invention removal/neutralization of immune suppressive cytokines may also be performed. This is illustrated in one publication that may be used as a guide for one of skill in the art. In the publication researchers showed that antibody-mediated TGFβ neutralization during radiation therapy effectively generates CD8(+) T-cell responses to multiple endogenous tumor antigens in poorly immunogenic mouse carcinomas. Generated T cells were effective at causing regression of irradiated tumors and nonirradiated lung metastases or synchronous tumors (abscopal effect). Gene signatures associated with IFNγ and immune-mediated rejection were detected in tumors treated with radiation therapy and TGFβ blockade in combination but not as single agents. Upregulation of programmed death (PD) ligand-1 and -2 in neoplastic and myeloid cells and PD-1 on intratumoral T cells limited tumor rejection, resulting in rapid recurrence. Addition of anti-PD-1 antibodies extended survival achieved with radiation and TGFβ blockade. Thus, TGFβ is a fundamental regulator of radiation therapy's ability to generate an in situ tumor vaccine. The combination of local radiation therapy with TGFβ neutralization offers a novel individualized strategy for vaccinating patients against their tumors [326].

In one aspect of the invention augmentation of immunity to cancer may be accomplished by addition of passive antibodies to cancer cells prior to, concomitant with, or subsequent to radiation therapy. The use of passive antibodies is described in the literature and these protocols may be utilized together with endothelial cell vaccination, such as vaccination with ValloVax, in order to augment therapeutic effects. For example combined treatment comprised of local irradiation and anti-neu antibody of tumor-bearing BALB/c mice significantly improved mouse survival (P<0.5), even though the tumor growth was similar to that of the irradiated-alone group. The combined treatment significantly reduced metastatic tumor masses in the lung and increased immune cell infiltration in primary tumor tissues. However, immune deficient nude mice with tumors did not exhibit prolonged survival in response to the combined treatment. Collectively, these results show that combined local irradiation and anti-neu antibody can elicit an immune-mediated abscopal effect to extend survival. Although the mechanism for abscopal effects induced by the combined treatment of radiation and anti-HER2/neu antibody was not elucidated, to our knowledge this is the first published study to describe the abscopal effect induced by the combination of local irradiation and the anti-HER2/neu antibody [327].

While the patent teaches enhancement of immunotherapy using immunization of endothelial cells modified in a manner to stimulate immunity to tumor endothelium, other means of abscopal effect stimulation involve non-immunological mechanisms. Mechanisms described below are discussed. In one study researchers examined whether abscopal effect was mediated through p53, a protein complex up-regulated in irradiated cells. Non-tumor-bearing legs of C57BL/6 (wild-type p53) and p53 null B6.129S2-Trp53(tm1Tyj) mice were irradiated to determine whether an abscopal effect could be observed against Lewis lung carcinoma (LLC) and T241 (fibrosarcoma) implanted at a distant site. In mice with wild-type p53, both LLC and T241 tumors implanted into the midline dorsum grew at a significantly slower rate when the leg of the animal was exposed to five 10-Gy fractions of radiation compared with sham-irradiated animals, suggesting that the abscopal effect is not tumor specific. When the radiation dose to the leg was reduced (twelve fractions of 2 Gy each), the inhibition of LLC tumor growth was decreased indicating a radiation-dose dependency for the abscopal effect. In contrast, when the legs of p53 null animals or wild-type p53 mice treated with pifithrin-alpha (a p53 blocker) were irradiated (five 10-Gy fractions), tumor growth was not delayed. These data implicate p53 as a key mediator of the radiation-induced abscopal effect and suggest that pathways downstream of p53 are important in eliciting this response [328].

Clinical use of combination checkpoint inhibitors for augmenting the abscopal effect have been described and incorporated by reference [329-332].

In some embodiments of the invention modification of neutrophil numbers and/or activity is performed in order to augment abscopal effect. This is further modified by placental vaccination. It has been observed that RT induced sterile inflammation with a rapid and transient infiltration of CD11b⁺Gr-1^{high+} neutrophils into the tumors. RT-recruited tumor-associated neutrophils (RT-Ns) exhibited an increased production of reactive oxygen species and induced apoptosis of tumor cells. Tumor infiltration of RT-Ns resulted in sterile inflammation and, eventually, the activation of tumor-specific cytotoxic T cells, their recruitment into the tumor site, and tumor regression. Finally, the concurrent administration of granulocyte colony-stimulating factor (G-CSF) enhanced RT-mediated antitumor activity by activating RT-Ns [333]. Augmentation of neutrophil activity may be performed by addition of various agents such as N-acetylcysteine intravenously at 0.2-200 micrograms per kilogram of body weight daily.

Intravenous vitamin C has been believed to induce anticancer responses through induction of oxidative stress in tumor cells. Combination of radiation and ascorbic acid has demonstrated augmentation of cancer cell death.

Cryosurgery has been demonstrate to stimulate immune responses as far back as 1969 in which a study was published demonstrating generation of aggulinating antibodies in patients with prostate cancer that underwent this procedure [334]. Similar results where obtain in 1974 in an animal model of cancer treatment by cryosurgery [335]. The ability of a localized induction of tumor cell death to induce a systemic antibody response was observed in a subsequent animal study in which an abscopal type systemic inhibition of tumor growth was observed subsequent to cryosurgery [336]. The systemic effect induced by cryosurgery was similar to the abscopal effect that is observed in cancer when localized radiation is used to induce tumor cell death [337-339]. Effects of radiation induced immunity may be amplified by macrophage activation [340].

In some embodiments of the invention dendritic cells are administered to the area of cryosurgery. Various means of generating dendritic cells are known in the art and are useful for the practice of the invention. For example, placental antigens may be used to pulse dendritic cells.

In one embodiment the invention teaches that administration of ValloVax, a placental endothelium based vaccine described in the following references [341-344], is capable of sensitizing tumors to treatment with cancer vaccines, said cancer vaccines comprising of either peptide vaccines, protein vaccines, cellular vaccines, or endogenous vaccines. Without being bound to theory, said cancer endothelial targeting vaccines are capable of specifically inducing inactivation of tumor endothelial mediated lymphocyte death, thus allowing for cancer killing T cells to specifically enter the tumor and mediate tumor cell death. As substitution for ValloVax, other types of endothelial progenitor cells (EPC) may be used to stimulate immunity to tumor endothelium. Said EPC, in one embodiment are a population of cells comprising cells having the surface marker CD44 [345], cells having the surface marker CD13 [346, 347], cells having the surface marker CD90 [345, 348-353], cells having the surface marker CD105 [346, 349, 354-360], cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s. Said EPC may be treated in a manner to mimic the tumor microenvironment, specifically, they may be grown under the acidic conditions in the tumor microenvironment and are incorporated by reference [361-374]. In one embodiment of the invention, endothelial progenitor cells, or products thereof, are cultured under conditions in GCN2 kinase is activated [375, 376], said conditions include culture in the presence of uncharged tRNA [377-380], tryptophan deprivation [381-383], arginine deprivation [384-389], asparagine deprivation [390-394], and glutamine deprivation [395, 396].

In some embodiments cells useful for generation of neurons, wherein said neurons will be implanted intratumorally will be umbilical cord mesenchymal stem cells. These may be derived from various areas of the umbilical cord. The cells of the invention are cultured under hypoxia, in one embodiment, cultured in order to induce and/or augment expression of chemokine receptors. One such receptor is CXCR-4. The population of cells, including population of umbilical cord mesenchymal cells, and more specifically, cells extracted from the subepithelial layer (SL) of the umbilical cord. A variety of techniques can be utilized to extract the isolated cells of the present disclosure from the SL, and any such technique that allows such extraction without significant damage to the cells is considered to be within the present scope. In one aspect, for example, a method of culturing stem cells from the SL of a mammalian umbilical cord can include dissecting the subepithelial layer from the umbilical cord. In one aspect, for example, umbilical cord tissue can be collected and washed to remove blood, Wharton's Jelly, and any other material associated with the SL. For example, in one non-limiting aspect the cord tissue can be washed multiple times in a solution of Phosphate-Buffered Saline (PBS) such as Dulbecco's Phosphate-Buffered Saline (DPBS). In some aspects the PBS can include a platelet lysate (i.e. 10% PRP lysate of platelet lysate). Any remaining Wharton's Jelly or gelatinous portion of the umbilical cord can then be removed and discarded. The remaining umbilical cord tissue (the SL) can then be placed interior side down on a substrate such that an interior side of the SL is in contact with the substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate. A variety of substrates are contemplated upon which the SL can be placed. In one aspect, for example, the substrate can be a solid polymeric material. One example of a solid polymeric material can include a cell culture dish. The cell culture dish can be made of a cell culture treated plastic as is known in the art. In one specific aspect, the SL can be placed upon the substrate of the cell culture dish without any additional pretreatment to the cell culture treated plastic. In another aspect, the substrate can be a semi-solid cell culture substrate. Such a substrate can include, for example, a semi-solid culture medium including an agar or other gelatinous base material. Following placement of the SL on the substrate, the SL is cultured in a suitable medium. In some aspects it is preferable to utilized culture media that is free of animal and human components or contaminants. The culture can then be cultured under either normoxic or hypoxic culture conditions for a period of time sufficient to establish primary cell cultures. (e.g. 3-7 days in some cases). After primary cell cultures have been established, the SL tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1.times. glutamine, 1.times. NEAA, and 0.1-20% PRP lysate or platelet lysate. Another example of suitable media can include a base medium of DMEM low glucose without phenol red, 1.times. glutamine, 1.times. NEAA, 1000 units of heparin and 20% PRP lysate or platelet lysate. In another example, cells can be cultured directly onto a semi-solid substrate of DMEM low glucose without phenol red, 1.times. glutamine, 1.times. NEAA, and 20% PRP lysate or platelet lysate. In a further example, culture media can include a low glucose medium (500-1000 mg/mL) containing 1.times. Glutamine, 1.times. NEAA, 1000 units of heparin. In some aspects, the glucose can be 1000-4000 mg/mL, and in other aspects the glucose can be high glucose at 4000-6000 mg/mL. These media can also include 0.1%-20% PRP lysate or platelet lysate. In yet a further example, the culture medium can be a semi-solid with the substitution of acid-citrate-dextrose ACD in place of heparin, and containing low glucose medium (500-1000 mg/mL), intermediate glucose medium (1000-4000 mg/mL) or high glucose medium (4000-6000 mg/mL), and further containing 1.times. Glutamine, 1.times. NEAA, and 0.1%-20% PRP lysate or platelet lysate. In some aspects, the cells can be derived, subcultured, and/or passaged using TrypLE. In another aspect, the cells can be derived, subcultured, and/or passaged without the use of TrypLE or any other enzyme.

In other embodiments of the invention, purified populations of regenerative cells can be obtained from the lining of a human umbilical cord. As used herein, “purified” means that at least 90% (e.g., 91, 92, 93, 94, 95, 96, 97, 98, or 99%) of the cells within the population are regenerative cells. As used herein, “regenerative cells” refers to mammalian cell. Within the context of the current invention regenerative cells can be isolated from umbilical cords obtained with informed consent. Typically, after an umbilical cord is obtained in a hospital or clinic, the cord is placed in a hypothermic preservation solution, such as FRS solution from Biolife Solutions (catalog #HTS-FRS) and stored at 4.degree. C. To begin isolating ULSCs, the hypothermic preservation solution can be removed by washing in a buffer, such as Hank's basic salt solution, that is free of Mg.sup.2+, Ca.sup.2+, and phenol free. The umbilical cord can be cut into cross sections in the presence of a buffer, and then the cross-sections can be cut longitudinally into two pieces while avoiding any venous or arterial tissue. If any blood is released into the buffer while cutting the cord, the contaminated buffer is replaced with fresh buffer. The longitudinal pieces of cord can be dissected to remove venous and arterial tissue such that the resulting cord lining (i.e., the gelatinous cord material) is substantially free of venous and arterial tissue. As used herein “substantially free of venous and arterial tissue” indicates that as much visible venous and arterial tissue has been removed as possible with manual dissection. Regenerative cells can be obtained from the dissected cord lining by culturing the longitudinal pieces of cord lining on a fibronectin coated solid substrate (e.g., a plastic culture device such as a chambered slide or culture flask). The gelatinous surface of the cord lining can be placed in contact with the fibronectin coated solid substrate while the upper surface (i.e., the surface not in contact with the fibronectin coated solid substrate) can be covered with a solid substrate such as a coverslip. Low glucose (i.e., .ltoreq.1 g/L glucose) growth medium can be added and the culture device incubated for a time sufficient for cells to migrate from the cord lining to the fibronectin coated solid substrate (e.g., 7 to 10 days). Unless otherwise indicated, cells are cultured at 37.degree. C. in a standard atmosphere that includes 5% CO.sub.2. Relative humidity is maintained at about 100%. After have adhered to the surface of the fibronectin coated solid substrate, the coverslip can be removed, and the adhered cells can be washed in a buffer such as phosphate-buffered saline (PBS). A growth medium that can be used for culturing Regenerative cells is low glucose Dulbecco's Modified Essential Media (DMEM) containing vitamins (choline chloride, D-Calcium pantothenate, Folic Acid, Nicotinamide, Pyridoxal hydrochloride, Riboflavin, Thiamine hydrochloride, and i-Inositol), and non-essential amino acids (glycine, L-alanine, L-Asparagine, L-Aspartic acid, L-Glutamic Acid, L-Proline, and L-Serine). Low glucose DMEM can be supplemented with 10% to 20% serum (e.g., fetal bovine serum (FBS) or human serum), one or more antibiotics (e.g., gentamycin, penicillin, or streptomycin), and glutamine or a stabilized dipeptide of L-alanyl-L-glutamine (e.g., GlutaMax from Invitrogen). In one embodiment, a growth medium can include low glucose DMEM containing vitamins and non-essential amino acids, 15% FBS, 1 to 3% antibiotic (e.g., 2% or 2.times. gentamycin), and 0.7 to 1.5% (e.g., 1%) of glutamine or a stabilized dipeptide of L-alanyl-L-glutamine. Such a growth medium can be further supplemented with 1 to 100 ng/mL of a growth factor (e.g., basic fibroblast growth factor (bFGF), leukemia inhibitory factor (LIF), or epidermal growth factor (EGF).

In some embodiments, a growth medium further includes insulin, transferrin, selenium, and sodium pyruvate. A particularly useful growth medium can include low glucose DMEM containing vitamins and non-essential amino acids, 15% serum, 1 to 3% antibiotic (e.g., 2% or 2.times. gentamycin), 0.7 to 1.5% of glutamine or a stabilized dipeptide of L-alanyl-L-glutamine (e.g., 1% or 1.times. GlutaMax), 1 to 100 ng/mL of a growth factor (e.g., 10 ng/mL bFGF and 10 ng/mL LIF), 0.1 mg/mL to 100 mg/mL of insulin (10 mg/mL), 0.1 mg/mL to 100 mg/mL of transferrin (e.g., 0.55 mg/mL transferring), 0.1 .mu.g/mL to 100 .mu.g/mL selenium (e.g., 0.5 .mu.g/mL selenium), and 0.5 to 1.5% sodium pyruvate (e.g., 1% sodium pyruvate). In some embodiments, such a growth medium further includes 0.05 .mu.g/mL to 100 .mu.g/mL of putrescine (e.g., 10 .mu.g/mL putrescine) and 10 ng/mL of EGF. For embodiments in which an animal free medium is desired, human serum (e.g., 15% human serum) can be used in place of fetal bovine serum.

In some embodiments of the invention, it is necessary to subculture regenerative cells, TrypZean (Sigma Chemical Co.) can be used to release cells from the solid substrate. The resulting cell suspension can be pelleted and washed with PBS, then seeded into cell culture flasks at approximately 1000 cells/cm.sup.2 in a growth medium. Clonal lines of Regenerative cells can be established by plating the cells at a high dilution and using cloning rings (e.g., from Sigma) to isolate single colonies originating from a single cell. Cells are obtained from within the cloning ring using trypsin then re-plated in one well of a multi-well plate (e.g., a 6-well plate). After cells reach >60% confluency (e.g., >70% confluency), the cells can be transferred to a larger culture flask for further expansion. Regenerative cells can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

Regenerative cells can be immunophenotypically characterized using known techniques. For example, the cells can be fixed (e.g., in paraformaldehyde), permeabilized, and reactive sites blocked (e.g., with serum albumin), then incubated with an antibody having binding affinity for a cell surface antigen. The antibody can be detectably labeled (e.g., fluorescently or enzymatically) or can be detected using a secondary antibody that is detectably labeled. In some embodiments, the cell surface antigens on Regenerative cells can be characterized using flow cytometry and fluorescently labeled antibodies.

Regenerative cells also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining)

Regenerative cellscan be cryopreserved by suspending the cells (e.g., up to 5.times.10.sup.6 cells/mL) in a cryopreservative such as dimethylsulfoxide (DMSO, typically 10%). In some embodiments, a freezing medium such as CryoStor from Biolife solutions is used to cryopreserve the cells. After adding cryopreservative, the cells can be frozen (e.g., to −90.degree. C.). In some embodiments, the cells are frozen at a controlled rate (e.g., controlled electronically or by suspending the cells in a bath of 70% ethanol and placed in the vapor phase of a liquid nitrogen storage tank. When the cells are chilled to −90.degree. C., they can be placed in the liquid phase of the liquid nitrogen storage tank for long term storage. Cryopreservation can allow for long-term storage of these cells for therapeutic use.

These cells isolated according to the above methodology may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In particular embodiments, cells of the invention lack expression of CD90, CD105 and CD34 but possess CD56, and/or CD73 expression.

In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell. Exosomes produced by cells described herein, in some embodiments lack expression of miR

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells-blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells-blood cells that can develop into several types of blood cells, but cannot develop into brain cells. At the end of the long chain of cell divisions that make up the embryo are “terminally differentiated” cells-cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

In one embodiment the invention teaches phenotypically defined MSC which can be isolated from the Wharton's jelly of umbilical cord segments and defined morphologically and by cell surface markers. By dissecting out the veins and arteries of cord segments and exposing the Wharton's jelly, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of cancer. The cells may be differentiated into neuronal phenotypes or may be administered in an undifferentiated manner. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric. acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

The cells of the invention may be useful for numerous types of indications. Below are some examples of indications and rationale that they may be used for

In one embodiment, cells of the invention may be utilized for treatment of Amyotrophic lateral sclerosis (ALS). This is a progressive neurodegenerative condition causing muscular atrophy and death within 3-5 years after its onset [397]. In the majority of patients (90%) the cause of ALS idiopathic, however in about 10% of the patients a familial form of the disease is presented [398]. Specific muscular degeneration is exclusive to motor neurons and begins focally and spreads, leading to weakness of limb, respiratory, and bulbar muscles. Immediately preceding death, there is a near total loss of limb and respiratory function, as well as a loss of the ability to chew, swallow, and speak. In the USA, ALS is defined as an “orphan disease,” with approximately 2 per 100,000 new cases per year and a prevalence of about 5 per 100,000 total cases each year [399]. In the United States [400] and Europe [401], ALS is diagnosed in about 1 in 500 to 1 in 1,000 adult deaths, implying that 500,000 people in the United States will develop this disease in their lifetimes. About 10% of ALS cases are inherited, usually as dominant traits [402]. Both familial ALS (fALS) and sporadic ALS (sALS) can develop concurrently with frontotemporal lobar dementia (FTLD). By contrast with the dementia of Alzheimer disease (AD), in which the cardinal finding is memory loss, FTLD is characterized by behavioral changes and progressive aphasia, sometimes accompanied by movement disorders. While AD involves prominent pathology in the hippocampus, the essential finding in FTLD is, as the name suggests, early atrophy of the frontal and temporal lobes. Four recurring themes have emerged from the pathological analysis of autopsied cases with sALS, fALS, or ALS-FTLD with diverse genetic causes. First, the motor neuron death usually entails deposition of aggregated proteins, often ubiquitinated and predominantly cytoplasmic. Second, in ALS, the levels and functions of RNA and RNA-binding proteins are abnormal. Aggregates of protein and RNA are detected both in motor neurons and non-neuronal cells, such as astrocytes and microglia. Third, most cases entail some disturbance of neuronal cytoskeletal architecture and function. Additionally, in almost all cases, motor neuron death is influenced by non-neuronal cells, including oligodendroglia and cells involved in neuroinflamation (e.g., astroglia and microglia).

We incorporate be reference trials of other types of MSC in order to provide guidance for one of skill in the art how to administer the cells of the current invention, and how to designed clinical trials based on previous types of cell therapies.

One of the first clinical interventions using mesenchymal stem cells in ALS was a report by Mazzin et al [403], who treated ALS patients with bone marrow ex vivo expanded MSC. Specifically, bone marrow collection was performed according to the standard procedure by aspiration from the posterior iliac crest. Ex vivo expansion of mesenchymal stem cells was induced according to Pittenger's protocol [404]. The cells were suspended in 2 ml of autologous cerebrospinal fluid and transplanted into the spinal cord by a micrometric pump injector. No patient manifested major adverse events such as respiratory failure or death. Minor adverse events were intercostal pain irradiation (4 patients) which was reversible after a mean period of three days after surgery, and leg sensory dysesthesia (5 patients) which was reversible after a mean period of six weeks after surgery. No modification of the spinal cord volume or other signs of abnormal cell proliferation were observed. The authors concluded by stating that it appears that the procedures of ex vivo expansion of autologous mesenchymal stem cells and of transplantation into the spinal cord of humans are safe and well tolerated by ALS patients. The same group reported 3 year follow up of the initial patients treated. Seven patients affected by definite ALS were enrolled in the study and two patients were treated for compassionate use. No patient manifested major adverse events such as respiratory failure or death. Minor adverse events were intercostal pain irradiation and leg sensory dysesthesia, both reversible after a mean period of 6 weeks. No modification of the spinal cord volume or other signs of abnormal cell proliferation were observed. A significant slowing down of the linear decline of the forced vital capacity was evident in four patients 36 months after MSCs transplantation [405]. An additional two studies where performed by the same group on 10 and 19 patients. The longest observation of treated patients was performed at 9 years after treatment. No long term adverse effects were detected and marginal therapeutic effects were seen [406, 407].

A study by an independent group evaluated the safety of two repeated intrathecal injections of autologous bone marrow (BM)-derived mesenchymal stromal cells (MSCs) in ALS patients. Eight patients with definite or probable ALS were enrolled. After a 3-month lead-in period, autologous MSCs were isolated two times from the BM at an interval of 26 days and were then expanded in vitro for 28 days and suspended in autologous cerebrospinal fluid. Of the 8 patients, 7 received 2 intrathecal injections of autologous MSCs (1×10(6) cells per kg) 26 days apart. Clinical or laboratory measurements were recorded to evaluate the safety 12 months after the first MSC injection. The ALS Functional Rating Scale-Revised (ALSFRS-R), the Appel ALS score, and forced vital capacity were used to evaluate the patients' disease status. One patient died before treatment and was withdrawn from the study. The death was not study related, and was attributable to natural progression of disease. With the exception of that patient, no serious adverse events were observed during the 12-month follow-up period. Most of the adverse events were self-limited or subsided after supportive treatment within 4 days. Decline in the ALSFRS-R score was not accelerated during the 6-month follow-up period. Two repeated intrathecal injections of autologous MSCs were safe and feasible throughout the duration of the 12-month follow-up period [408]. A subsequent study from Belarus utilized autologous mesenchymal stem cells were injected intravenously (intact cells) or via lumbar puncture (cells committed to neuronal differentiation). Evaluation of the results of cell therapy after 12-month follow-up revealed slowing down of the disease progression, as assessed by ALSFRS-R score was observed in 10 patients that were treated with cells. In comparison, in a control group that was matched for age and disease status, no slowing down of progression was observed. The Control group consisted of 15 patients. The study reported no adverse effects associated with administration of mesenchymal stem cells intravenously or intrathecally [409].

Although the above clinical studies support a possible benefit for reducing progression of ALS, as determined by slowing down advancement of disease as quantified by measures such as ALSFRS-R score, none of the studies reversed the disease progression. One possible reason is that when the stem cells where introduced via lumbar injection (intrathecally), the cells hypothetically would tend to sink downwards rather than ascending to the brain and cervical and thoracic spinal cord. Therefore, a study by Baek et al. [410], assessed the ability to utilize intraventricular injections directly into the brain by using an Ommaya reservoir to administer cells. The Ommaya reservoir is catheter system that is typically used for the delivery of drugs directly into the ventricles of the brain. It consists of a catheter in one lateral ventricle attached to a reservoir implanted under the scalp. It is typically used to treat brain tumors, leukemia/lymphoma or leptomeningeal disease, as well as for intracerebroventricular (ICV) injection of morphine [411]. Others have previously used the Ommaya reservoir to deliver cell therapy into the brain. To give an indication of the relative safety of this approach, in one study in glioma patients, autologous tumor infiltrating lymphocytes that were expanded ex vivo we administered in 6 patients by use of the Ommaya reservoir. One patient had complete response, 2 had partial responses, and 3 succumbed to disease. Most interestingly, no serious adverse effects were noted, despite the fact that activated lymphocytes were directly injected into the brain, an area typically classified as very sensitive in inflammation [412]. With the rational this, and other studies have successfully administered cells in the brain [413-415], and mesenchymal stem cells are generally considered anti-inflammatory, Baek et al attempted to adopt this procedure for use in ALS in a patient. Bone marrow mesenchymal stem cells were isolated from the bone marrow of a male patient with ALS who underwent insertion of an Ommaya reservoir. Expanded MSCs (hBM-MSCs: dose of 1×106 cells/kg) were suspended in autologous CSF and directly transplanted into the ALS patient's lateral ventricle via the Ommaya reservoir. Clinical, laboratory, and radiographic evaluation of the patient revealed no serious adverse effects related to the stem cell therapy. The authors concluded that intraventricular injection with an optimized number of cells is safe, and is a potential route for stem cell therapy in patients with ALS. Intraventricular injection via an Ommaya reservoir makes repetitive injection of stem cells easy and reliable even in far advanced ALS patients. Unfortunately, no discussion on impact on disease progression was given in the publication.

In another attempt to increase therapeutic efficacy of mesenchymal stem cells in ALS, researchers have explored in vitro means of augmenting neurotrophic factor production by manipulation of culture conditions. A series of studies from the Hadassah Medical Center in Jerusalem, Israel attempted to treat ALS by in vitro manipulated MSC that are validated to produce higher amounts of neurotrophic factors. In the studies, all patients were followed up for 3 months before transplantation and 6 months after transplantation. In the phase 1/2 part of the trial, 6 patients with early-stage ALS were injected intramuscularly (IM) and 6 patients with more advanced disease were transplanted intrathecally (IT). In the second stage, a phase 2a dose-escalating study, 14 patients with early-stage ALS received a combined IM and IT transplantation of autologous MSC-NTF cells. It was reported that among the 12 patients in the phase 1/2 trial and the 14 patients in the phase 2a trial aged 20 and 75 years, the administration of mesenchymal stem cells was found to be safe and well tolerated over the study follow-up period. Most of the adverse effects were mild and transient, not including any treatment-related serious adverse event. The rate of progression of the forced vital capacity and of the ALSFRS-R score in the IT (or IT+IM)-treated patients was reduced (from −5.1% to −1.2%/month percentage predicted forced vital capacity, P<0.04 and from −1.2 to 0.6 ALSFRS-R points/month, P=0.052) during the 6 months following MSC-NTF cell transplantation vs the pretreatment period. Of these patients, 13 (87%) were defined as responders to either ALSFRS-R or forced vital capacity, having at least 25% improvement at 6 months after treatment in the slope of progression. A subsequent report on MSC-NTF cells described observations after these cells where delivered by combined intrathecal and intramuscular administration to participants with amyotrophic lateral sclerosis (ALS) in a phase 2 randomized controlled trial. The study enrolled 48 participants randomized 3:1 (treatment: placebo). After a 3-month pretransplant period, participants received 1 dose of MSC-NTF cells (n=36) or placebo (n=12) and were followed for 6 months. CSF was collected before and 2 weeks after transplantation. The study met its primary safety endpoint. The rate of disease progression (Revised ALS Functional Rating Scale [ALSFRS-R] slope change) in the overall study population was similar in treated and placebo participants. In a prespecified rapid progressor subgroup (n=21), rate of disease progression was improved at early time points (p<0.05). To address heterogeneity, a responder analysis showed that a higher proportion of treated participants experienced >1.5 points/month ALSFRS-R slope improvement compared to placebo at all time points, and was significant in rapid progressors at 4 and 12 weeks (p=0.004 and 0.046, respectively). CSF neurotrophic factors increased and CSF inflammatory biomarkers decreased in treated participants (p<0.05) post-transplantation. CSF monocyte chemoattractant protein-1 levels correlated with ALSFRS-R slope improvement up to 24 weeks (p<0.05). Thus the authors summarized that a single-dose transplantation of MSC-NTF cells is safe and demonstrated early promising signs of efficacy. This establishes a clear path forward for a multidose randomized clinical trial of intrathecal autologous MSC-NTF cell transplantation in ALS [416].

In conclusion, although the main utility of the cells is cancer treatment, protocols disclosed can be useful for the use of the current invention in the treatment of ALS, there are means of enhancing efficacy. The cells of the current invention allow for a) increasing frequency of dosing, in part because the cells are smaller in size than typical MSC, higher number of cells may be given without fear of lung pathology; b) further augmentation of mesenchymal stem cell regenerative activity through culture conditions or gene manipulation, for example, addition of neurotrophic factors in vitro to enhance activity may be performed; c) the use of other growth/neurotrophic factors as adjuvants. For example, it is known that endogenous neuronal stem cells exist, whose activity may be modified by administration of various compounds, some compounds already clinically available. Well-known examples of approved drugs that augment endogenous neural stem cell activity include lithium [417, 418], valproic acid [419], and human chorionic gonadotropin [420], these agents are disclosed for use within the context of the current invention. Interestingly, the stem cell modifier combination of lithium and valproic acid was already assessed on its own in a small trial which suggested some possible efficacy. The study recruited 18 patients that were treated with the combination and compared them to 31 controls that were carefully paired by age, gender, evolution rate and time of the disease, who never received treatment with lithium and/or valproate. Assessment of disease by ALSFRS-R was performed before treatment (baseline), 1 month after treatment, and every 4 months until the outcome (death or an adverse event). The investigators reported that lithium and valproate cotreatment significantly increased survival, and this treatment also exerted neuroprotection in our patients because all biochemical markers reached levels normal levels in the ALS patients that were treated. The biochemical markers were Cu/Zn superoxide dismutase and glutathione peroxidase activity, and reduced glutathione [421].

### EXAMPLES

### Example 1: Reversion of IL-10 Induced Immune Suppression by Nerve Growth Factor in Context of MCF-7 Breast Cancer

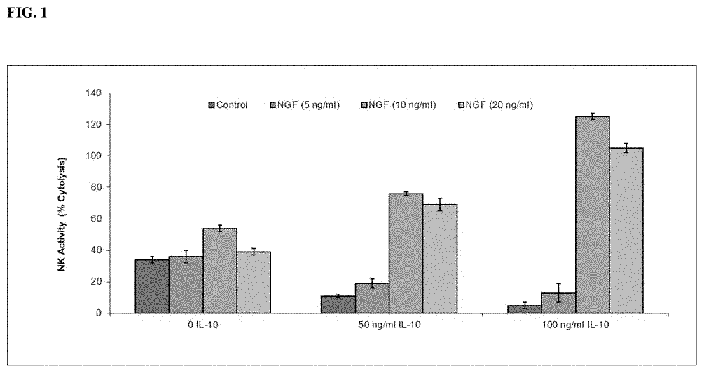

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of interleukin-10 and NGF was performed at each concentration indicated. Conditioned media was extracted after 4 days of culture and incubated with primary natural killer cells and cytotoxic activity was assessed against K-562 cell lines. As observed below, NGF resulted in inhibition of IL-10 induced suppression of NK cell activity. Results are shown in FIG. 1.

### Example 2: Reversion of VEGF Induced Immune Suppression by Nerve Growth Factor in Context of MCF-7 Breast Cancer

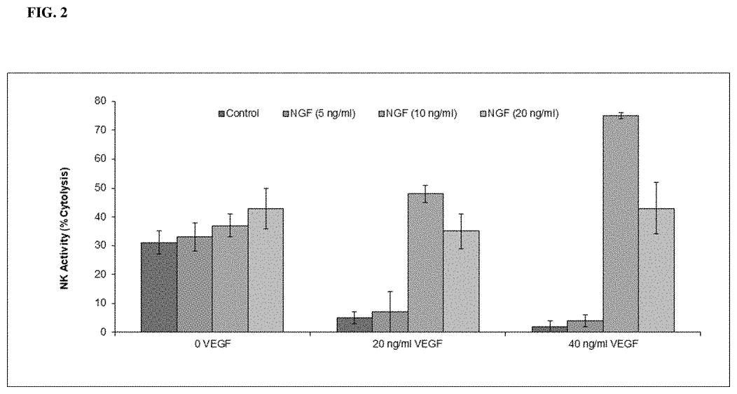

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of VEGF and NGF was performed at each concentration indicated. Conditioned media was extracted after 4 days of culture and incubated with primary natural killer cells and cytotoxic activity was assessed against K-562 cell lines. As observed below, NGF resulted in inhibition of VEGF induced suppression of NK cell activity. Results are shown in FIG. 2.

### Example 3: Reversion of Soluble HLA-G Induced Immune Suppression by Nerve Growth Factor in Context of MCF-7 Breast Cancer

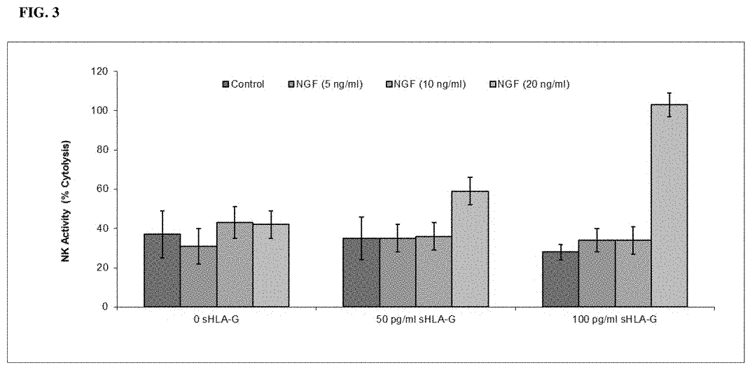

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of soluble HLA-G and NGF was performed at each concentration indicated. Conditioned media was extracted after 4 days of culture and incubated with primary natural killer cells and cytotoxic activity was assessed against K-562 cell lines. As observed below, NGF resulted in inhibition of HLA-G induced suppression of NK cell activity. Results are shown in FIG. 3.

### Example 4: Reversion of TGF-Beta Induced Immune Suppression by Nerve Growth Factor in Context of MCF-7 Breast Cancer

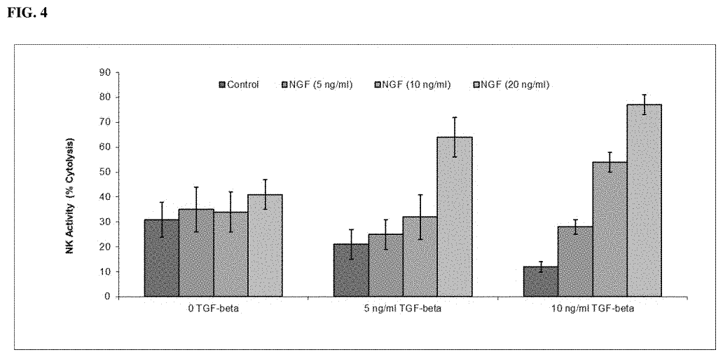

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of TGF-beta and NGF was performed at each concentration indicated. Conditioned media was extracted after 4 days of culture and incubated with primary natural killer cells and cytotoxic activity was assessed against K-562 cell lines. As observed below, NGF resulted in inhibition of TGF-beta induced suppression of NK cell activity. Results are shown in FIG. 4.

### Example 5: Reversion of IL-10 Induced Immune Suppression by BDNF in Context of MCF-7 Breast Cancer

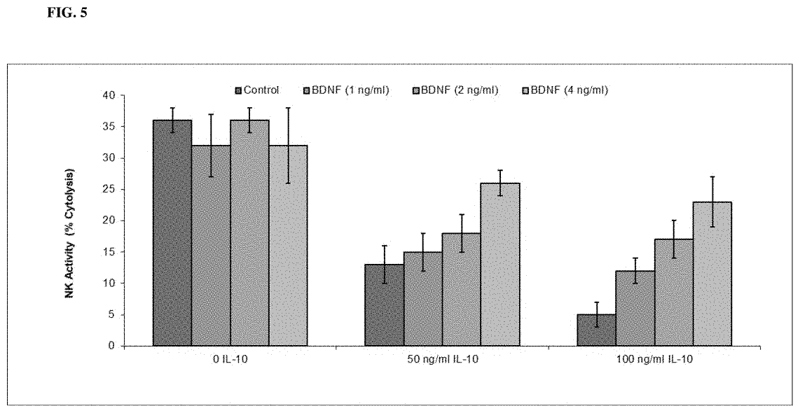

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of interleukin-10 and BDNF was performed at each concentration indicated. Conditioned media was extracted after 4 days of culture and incubated with primary natural killer cells and cytotoxic activity was assessed against K-562 cell lines. As observed below, BDNF resulted in inhibition of IL-10 induced suppression of NK cell activity. Results are shown in FIG. 5.

### Example 6: Reversion of VEGF Induced Immune Suppression by BDNF in Context of MCF-7 Breast Cancer

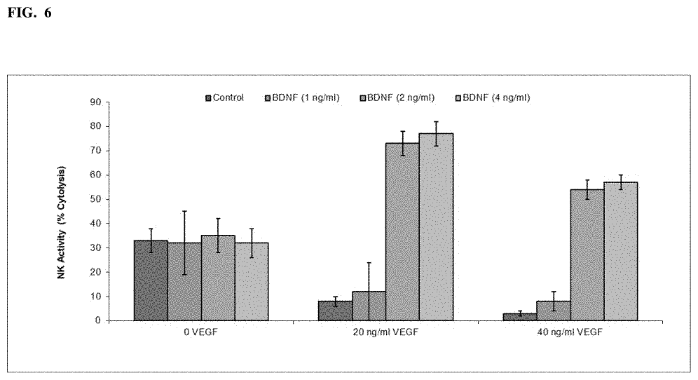

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of VEGF and BDNF was performed at each concentration indicated. Conditioned media was extracted after 4 days of culture and incubated with primary natural killer cells and cytotoxic activity was assessed against K-562 cell lines. As observed below, BDNF resulted in inhibition of VEGF induced suppression of NK cell activity. Results are shown in FIG. 6.

### Example 7: Reversion of Soluble HLA-G Induced Immune Suppression by BDNF in Context of MCF-7 Breast Cancer

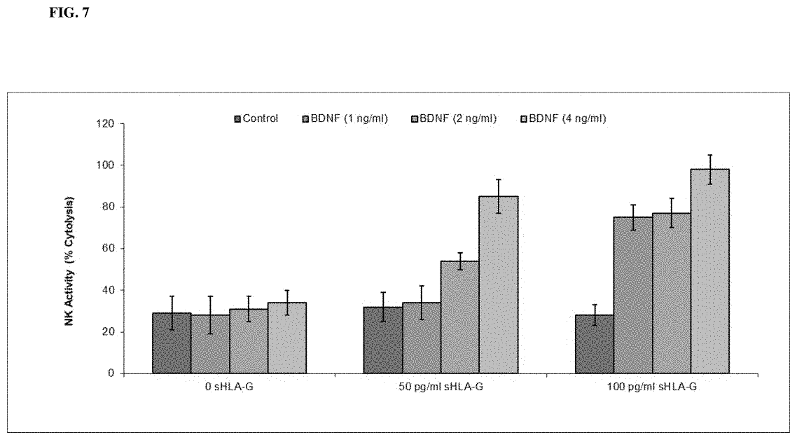

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of soluble HLA-G and BDNF was performed at each concentration indicated. Conditioned media was extracted after 4 days of culture and incubated with primary natural killer cells and cytotoxic activity was assessed against K-562 cell lines. As observed below, BDNF resulted in inhibition of HLA-G induced suppression of NK cell activity. Results are shown in FIG. 7.

### Example 8: Reversion of TGF-beta Induced Immune Suppression by BDNF in Context of MCF-7 Breast Cancer

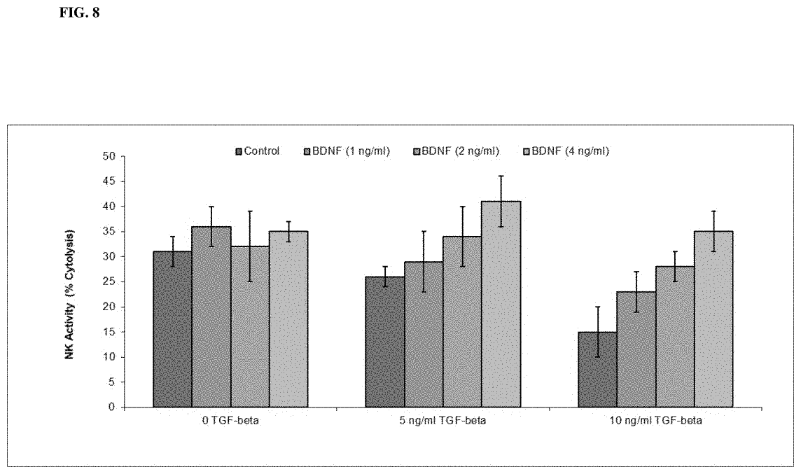

In order to replicate the tumor microenvironment, one to one cultures of human monocytes (obtained from American Type Tissue Culture, Manassas Va.) with MCF-7 triple negative breast cancer cells. Cultures were performed in 12 well plates at a concentration of 500,000 of each cell. Addition of TGF-beta and BDNF was performed at each concentration indicated. Conditioned media was extracted after 4 days of culture and incubated with primary natural killer cells and cytotoxic activity was assessed against K-562 cell lines. As observed below, BDNF resulted in inhibition of TGF-beta induced suppression of NK cell activity. Results are shown in FIG. 8.

### Example 9: Suppression of B16 Melanoma Growth by Administration of Neural Progenitor Cells

Umbilical cord mesenchymal stem cells were purchased from ATCC and grown according to the manufacturer's instructions. Cells of 1×104 cells/100 μL were plated into low-attachment plastic 96-well plates (Thermo Fisher Scientific) in neural stem cell medium composed of DMEM/F12 (Invitrogen), B-27 supplement without vitamin A (Invitrogen), 20 ng/mL epidermal growth factor (EGF, R&D Systems, Minneapolis, Minn.), 10 ng/mL fibroblast growth factor-2 (FGF-2, R&D Systems), and 10 ng/mL recombinant mouse leukemia inhibitory factor (Millipore) at 37° C. in 5% C02. Half the amount of medium was renewed twice a week for 7-10 days to generate spheres.

For neurogenic cell differentiation, the neurospheres were plated into low-attachment plastic 96-well dishes incubated at 37° C. for 24 h with alpha-modified minimum essential medium (α-MEM, Invitrogen) containing 1 mM β-mercaptoethanol (OME). The media were removed, washed with PBS (pH 7.4), and replaced with new media consisting of α-MEM, 10% FBS, and 35 ng/mL all-trans-retinoic acid (RA, Sigma-Aldrich) for 3 days. Neurospheres were washed with PBS and transferred to fibronectin-coated 8-well chamber slides (BD Biosciences) containing α-MEM, 10% FBS, 5 μM forskolin (FSK, Sigma-Aldrich), 20 ng/mL EGF, 10 ng/mL FGF-2, and 10 ng/mL human brain-derived neurotrophic factor (BDNF, Sigma-Aldrich) for 7 days. For glial cell differentiation, the neurospheres were plated into low-attachment plastic 96-well dishes incubated at 37° C. for 24 h with α-MEM containing 1 mM PME. The media were then removed, washed with PBS (pH 7.4), and replaced with new media of α-MEM containing 1 mM PME and 35 ng/mL RA for 3 days. Neurospheres were washed with PBS and transferred to fibronectin-coated 8-well chamber slides containing α-MEM, 10% FBS, M FSK, 10 ng/mL FGF-2, 5 ng/mL PDGF-BB (R&D Systems), and 200 ng/mL neuregulin 1-01/heregulin 101 EGF domain (R&D Systems) for 7 days. Samples were then fixed with 4% paraformaldehyde at 4° C. for 30 min and permeabilized with 0.5% Triton X-100 in TBS at room temperature for 30 min followed by an incubation in blocking buffer (10% goat serum, 1% bovine serum albumin in TBS) at room temperature for 1 h. Samples were incubated overnight at 4° C. with the following primary antibodies: mouse anti-microtubule-associated protein 2 (MAP2, clone HM-2, 1:100; Sigma-Aldrich), mouse anti-neurofilament 200 (NF200, 1:50; Sigma-Aldrich), mouse anti-β III tubulin (Clone TU-20, 1:100; Abcam, Cambridge, UK), mouse anti-oligodendrocyte marker 04 (clone 04, 1:50, R&D Systems), and mouse anti-glial fibrillary acidic protein (GFAP, clone G-A-5, 1:200, Sigma-Aldrich).

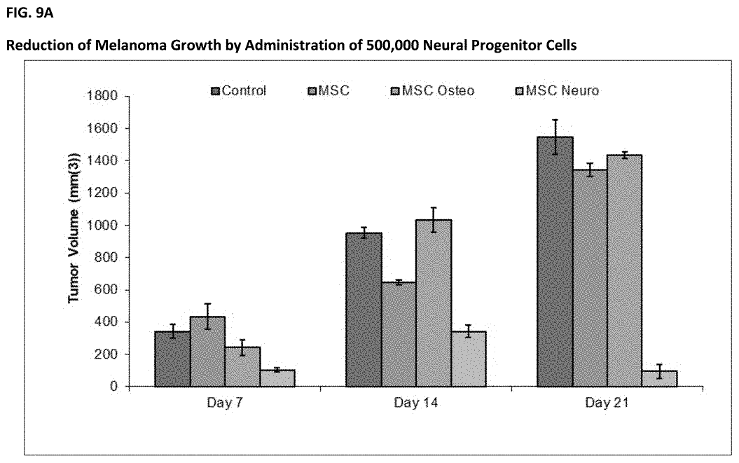

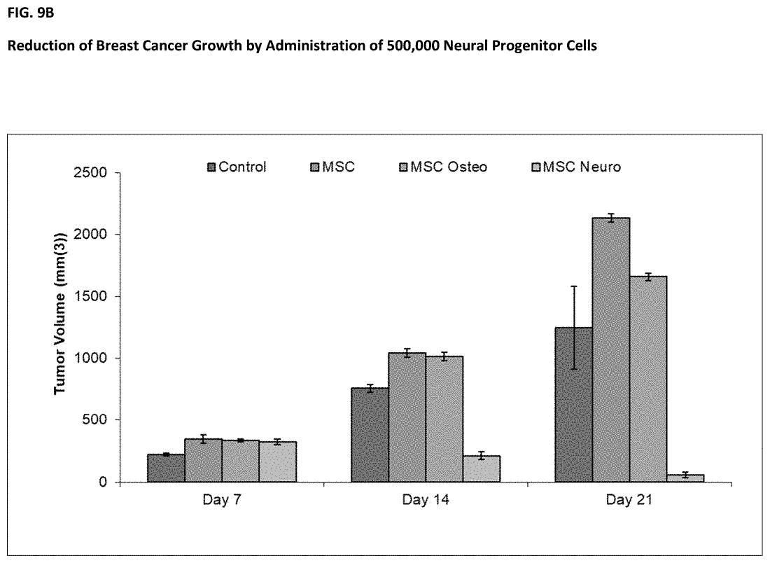

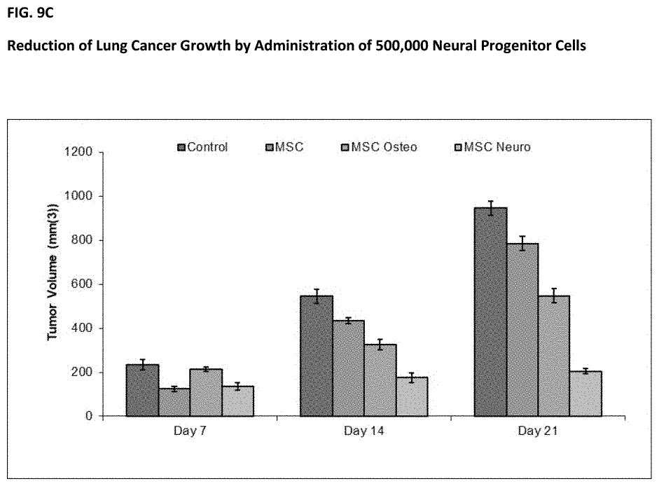

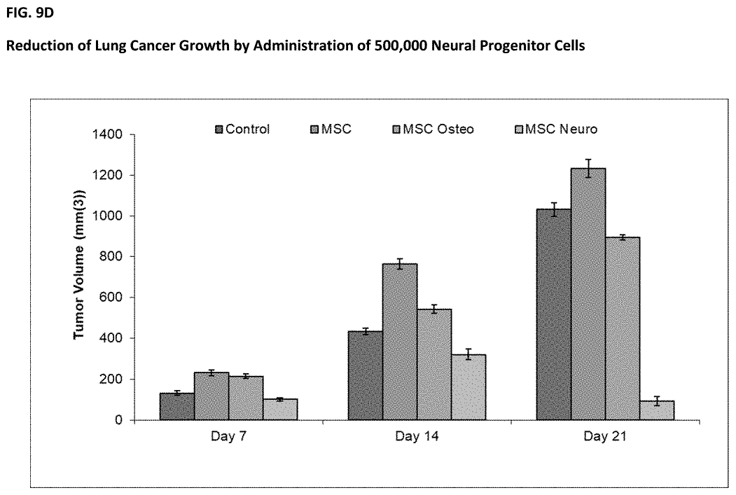

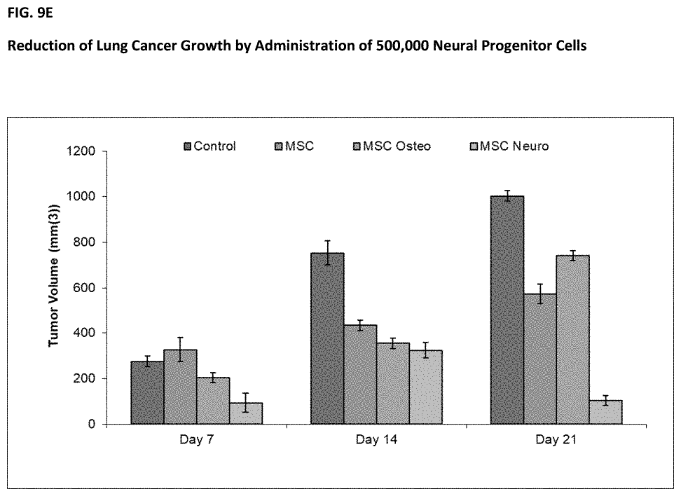

Mice were injected with B16 melanoma (FIG. 9A), 4T1 breast cancer (FIG. 9B). LLC lung cancer (FIG. 9C), C2-a glioma (FIG. 9D), and CD26 (FIG. 9E). Results are shown in FIGS. 9A-9E.

### REFERENCES

1. Rosenberg, S. A., J. C. Yang, and N. P. Restifo, Cancer immunotherapy: moving beyond current vaccines. Nat Med, 2004. 10(9): p. 909-15.
2. Hsu, F. J., et al., Tumor-specific idiotype vaccines in the treatment of patients with B-cell lymphoma—long-term results of a clinical trial. Blood, 1997. 89(9): p. 3129-35.
3. Neelapu, S. S., et al., Phase III randomized trial of patient-specific vaccination for previously untreated patients with follicular lymphoma in first complete remission: protocol summary and interim report. Clin Lymphoma, 2005. 6(1): p. 61-4.
4. Rosenberg, S. A., J. C. Yang, and N. P. Restifo, Reply-Cancer Vaccines: Pessimism in Check. Nat Med, 2004. 10(12): p. 1279-80.
5. Angelopoulou, K., et al., p53 gene mutation, tumor p53 protein overexpression, and serum p53 autoantibody generation in patients with breast cancer. Clin Biochem, 2000. 33(1): p. 53-62.
6. Disis, M. L., et al., Generation of T-cell immunity to the HER-2/neu protein after active immunization with HER-2/neu peptide-based vaccines. J Clin Oncol, 2002. 20(11): p. 2624-32.
7. Disis, M. L., et al., Generation of immunity to the HER-2/neu oncogenic protein in patients with breast and ovarian cancer using a peptide-based vaccine. Clin Cancer Res, 1999. 5(6): p. 1289-97.
8. Goydos, J. S., et al., A phase I trial of a synthetic mucin peptide vaccine. Induction of specific immune reactivity in patients with adenocarcinoma. J Surg Res, 1996. 63(1): p. 298-304.
9. Sandmaier, B. M., et al., Evidence of a cellular immune response against sialyl-Tn in breast and ovarian cancer patients after high-dose chemotherapy, stem cell rescue, and immunization with Theratope STn-KLH cancer vaccine. J Immunother, 1999. 22(1): p. 54-66.
10. Tilkin, A. F., et al., Primary proliferative T cell response to wild-type p53 protein in patients with breast cancer. Eur J Immunol, 1995. 25(6): p. 1765-9.
11. Jager, D., et al., Identification of tumor antigens as potential target antigens for immunotherapy by serological expression cloning. Cancer Immunol Immunother, 2004. 53(3): p. 144-7.
12. Ko, B. K., et al., Clinical studies of vaccines targeting breast cancer. Clin Cancer Res, 2003. 9(9): p. 3222-34.
13. Musselli, C., et al., Reevaluation of the cellular immune response in breast cancer patients vaccinated with MUC. Int J Cancer, 2002. 97(5): p. 660-7.
14. Tsuboi, A., et al., Enhanced induction of human WT1-specific cytotoxic T lymphocytes with a 9-mer WT1 peptide modified at HLA-A*2402-binding residues. Cancer Immunol Immunother, 2002. 51(11-12): p. 614-20.
15. Gaffey, M. J., et al., Medullary carcinoma of the breast. Identification of lymphocyte subpopulations and their significance. Mod Pathol, 1993. 6(6): p. 721-8.
16. Harris, J. R., M. Marrow, and G. Bonadonna, Cancer of the Breast, in CANCER: Principles and Practice of Oncology, S. H. V T Devita, S A Rosenberg, Editor. 1993, J. B. Lippincott Co.: Philadelphia, Pa.
17. Ridolfi, R. L., et al., Medullary carcinoma of the breast: a clinicopathologic study with 10 yearfollow-up. Cancer, 1977. 40(4): p. 1365-85.
18. Cohen, E. P. and T. S. Kim, Neoplastic cells that express low levels of MHC class I determinants escape host immunity. Semin Cancer Biol, 1994. 5(6): p. 419-28.
19. June, C. H., et al., The B7 and CD28 receptor families. Immunol Today, 1994. 15(7): p. 321-31.
20. Sinha, P., V. K. Clements, and S. Ostrand-Rosenberg, Interleukin-13-regulated M2 macrophages in combination with myeloid suppressor cells block immune surveillance against metastasis. Cancer Res, 2005. 65(24): p. 11743-51.
21. Agrawal, B., et al., Cancer-associated MUC1 mucin inhibits human T-cell proliferation, which is reversible by IL-2. Nat Med, 1998. 4(1): p. 43-9.
22. Hiltbold, E. M., et al., The mechanism of unresponsiveness to circulating tumor antigen MUC1 is a block in intracellular sorting and processing by dendritic cells. J Immunol, 2000. 165(7): p. 3730-41.
23. Kano, M., et al., Tanycyte-Like Cells Derived From Mouse Embryonic Stem Culture Show Hypothalamic Neural Stem/Progenitor Cell Functions. Endocrinology, 2019. 160(7): p. 1701-1718.
24. Jemal, A., et al., Cancer statistics, 2007. C A Cancer J Clin, 2007. 57(1): p. 43-66.
25. Cutuli, B., J. Bernier, and P. Poortmans, Radiotherapy in DCIS, an underestimated benefit? Radiother Oncol, 2014. 112(1): p. 1-8.
26. Barnes, N. L., et al., Absence of HER4 expression predicts recurrence of ductal carcinoma in situ of the breast. Clin Cancer Res, 2005. 11(6): p. 2163-8.
27. Czerniecki, B. J., et al., Targeting HER-2/neu in early breast cancer development using dendritic cells with staged interleukin-12 burst secretion. Cancer Res, 2007. 67(4): p. 1842-52.
28. Koski, G. K., et al., A novel dendritic cell-based immunization approach for the induction of durable Th1-polarized anti-HER-2/neu responses in women with early breast cancer. J Immunother, 2012. 35(1): p. 54-65.
29. Sharma, A., et al., HER-2 pulsed dendritic cell vaccine can eliminate HER-2 expression and impact ductal carcinoma in situ. Cancer, 2012. 118(17): p. 4354-62.
30. Fracol, M., et al., Response to HER-2 pulsed DC1 vaccines is predicted by both HER-2 and estrogen receptor expression in DCIS. Ann Surg Oncol, 2013. 20(10): p. 3233-9.
31. Brossart, P., et al., Induction of cytotoxic T-lymphocyte responses in vivo after vaccinations with peptide-pulsed dendritic cells. Blood, 2000. 96(9): p. 3102-8.
32. Morse, M. A., et al., HER2 dendritic cell vaccines. Clin Breast Cancer, 2003. 3 Suppl 4: p. S164-72.
33. Triozzi, P. L., et al., Intratumoral injection of dendritic cells derived in vitro in patients with metastatic cancer. Cancer, 2000. 89(12): p. 2646-54.
34. Pedersen, A. E., et al., Phenotypic and functional characterization of clinical grade dendritic cells generated from patients with advanced breast cancer for therapeutic vaccination. Scand J Immunol, 2005. 61(2): p. 147-56.
35. Svane, I. M., et al., Vaccination with p53 peptide-pulsed dendritic cells is associated with disease stabilization in patients with p53 expressing advanced breast cancer; monitoring of serum YKL-40 and IL-6 as response biomarkers. Cancer Immunol Immunother, 2007. 56(9): p. 1485-99.
36. Trounson, A., Human embryonic stem cell derivation and directed differentiation. Ernst Schering Res Found Workshop, 2005(54): p. 27-44.
37. Sasai, Y., Directed differentiation of neural and sensory tissues from embryonic stem cells in vitro. Ernst Schering Res Found Workshop, 2005(54): p. 101-9.
38. Peng, H. and G. Chen, Neural precursors derived from human embryonic stem cells. Sci China C Life Sci, 2005. 48(3): p. 295-9.
39. Takagi, Y., et al., Survival and differentiation of neural progenitor cells derived from embryonic stem cells and transplanted into ischemic brain. J Neurosurg, 2005. 103(2): p. 304-10.
40. Bouhon, I. A., et al., Neural differentiation of mouse embryonic stem cells in chemically defined medium. Brain Res Bull, 2005. 68(1-2): p. 62-75.
41. Joannides, A. J., et al., A scaleable and defined system for generating neural stem cells from human embryonic stem cells. Stem Cells, 2007. 25(3): p. 731-7.
42. Guillaume, D. J., et al., Human embryonic stem cell-derived neural precursors develop into neurons and integrate into the host brain. J Neurosci Res, 2006. 84(6): p. 1165-76.
43. Buhnemann, C., et al., Neuronal differentiation of transplanted embryonic stem cell-derived precursors in stroke lesions of adult rats. Brain, 2006. 129(Pt 12): p. 3238-48.
44. Wobus, A. M., R. Grosse, and J. Schoneich, Specific effects of nerve growth factor on the differentiation pattern of mouse embryonic stem cells in vitro. Biomed Biochim Acta, 1988. 47(12): p. 965-73.
45. Parada, L. F., et al., The Trk family of tyrosine kinases: receptors for NGF-related neurotrophins. Cold Spring Harb Symp Quant Biol, 1992. 57: p. 43-51.
46. Kato, M., et al., Hepatocyte growth factor promotes neuronal differentiation of neural stem cells derived from embryonic stem cells. Neuroreport, 2004. 15(1): p. 5-8.
47. Buytaert-Hoefen, K. A., E. Alvarez, and C. R. Freed, Generation of tyrosine hydroxylase positive neurons from human embryonic stem cells after coculture with cellular substrates and exposure to GDNF. Stem Cells, 2004. 22(5): p. 669-74.
48. Lee, C. S., et al., Neurotrophin and GDNF family ligands promote survival and alter excitotoxic vulnerability of neurons derived from murine embryonic stem cells. Exp Neurol, 2005. 191(1): p. 65-76.
49. Kitagawa, A., et al., Lecithinized brain-derived neurotrophic factor promotes the differentiation of embryonic stem cells in vitro and in vivo. Biochem Biophys Res Commun, 2005. 328(4): p. 1051-7.
50. Kim, B. K., et al., Neurogenic effect of vascular endothelial growth factor during germ layer formation of human embryonic stem cells. FEBS Lett, 2006. 580(25): p. 5869-74.
51. Benzing, C., et al., Neural conversion of human embryonic stem cell colonies in the presence of fibroblast growth factor-2. Neuroreport, 2006. 17(16): p. 1675-81.
52. Nurcombe, V., et al., MK: a pluripotential embryonic stem-cell-derived neuroregulatory factor. Development, 1992. 116(4): p. 1175-83.
53. Castro-Obregon, S. and L. Covarrubias, Role of retinoic acid and oxidative stress in embryonic stem cell death and neuronal differentiation. FEBS Lett, 1996. 381(1-2): p. 93-7.
54. Guan, K., et al., Embryonic stem cell-derived neurogenesis. Retinoic acid induction and lineage selection of neuronal cells. Cell Tissue Res, 2001. 305(2): p. 171-6.
55. Bain, G., et al., Embryonic stem cells express neuronal properties in vitro. Dev Biol, 1995. 168(2): p. 342-57.
56. Bain, G., et al., Retinoic acid promotes neural and represses mesodermal gene expression in mouse embryonic stem cells in culture. Biochem Biophys Res Commun, 1996. 223(3): p. 691-4.
57. Gajovic, S., et al., Retinoic acid mediates Pax6 expression during in vitro differentiation of embryonic stem cells. Differentiation, 1997. 62(4): p. 187-92.
58. Okada, Y., et al., Retinoic-acid-concentration-dependent acquisition of neural cell identity during in vitro differentiation of mouse embryonic stem cells. Dev Biol, 2004. 275(1): p. 124-42.
59. Rolletschek, A., et al., Differentiation of embryonic stem cell-derived dopaminergic neurons is enhanced by survival-promoting factors. Mech Dev, 2001. 105(1-2): p. 93-104.
60. Dinsmore, J., et al., Embryonic stem cells differentiated in vitro as a novel source of cells for transplantation. Cell Transplant, 1996. 5(2): p. 131-43.
61. Murray, P. and D. Edgar, The regulation of embryonic stem cell differentiation by leukaemia inhibitory factor (LIF). Differentiation, 2001. 68(4-5): p. 227-34.
62. Bonaguidi, M. A., et al., LIF and BMP signaling generate separate and discrete types of GFAP-expressing cells. Development, 2005. 132(24): p. 5503-14.
63. Nakai, S., et al., The POU domain transcription factor Brn-2 is required for the determination of specific neuronal lineages in the hypothalamus of the mouse. Genes Dev, 1995. 9(24): p. 3109-21.
64. Chung, S., et al., The homeodomain transcription factor Pitx3 facilitates differentiation of mouse embryonic stem cells into AHD2-expressing dopaminergic neurons. Mol Cell Neurosci, 2005. 28(2): p. 241-52.
65. Veal, E., et al., The secreted glycoprotein CREG enhances differentiation of NTERA-2 human embryonal carcinoma cells. Oncogene, 2000. 19(17): p. 2120-8.
66. Anderson, K. D., et al., Overexpression of HuD accelerates neurite outgrowth and increases GAP-43 mRNA expression in cortical neurons and retinoic acid-induced embryonic stem cells in vitro. Exp Neurol, 2001. 168(2): p. 250-8.
67. Lee, M. W., et al., Restricted expression of LUZP in neural lineage cells: a study in embryonic stem cells. J Biomed Sci, 2001. 8(6): p. 504-11.
68. Nakayama, T., T. Momoki-Soga, and N. Inoue, Astrocyte-derived factors instruct differentiation of embryonic stem cells into neurons. Neurosci Res, 2003. 46(2): p. 241-9.
69. Zhou, Y. F., et al., An experimental study on astrocytes promoting production of neural stem cells derived from mouse embryonic stem cells. Chin Med J (Engl), 2005. 118(23): p. 1994-9.
70. Roy, N. S., et al., Functional engraftment of human ES cell-derived dopaminergic neurons enriched by coculture with telomerase-immortalized midbrain astrocytes. Nat Med, 2006. 12(11): p. 1259-68.
71. Kornyei, Z., et al., Humoral and contact interactions in astroglia/stem cell co-cultures in the course of glia-induced neurogenesis. Glia, 2005. 49(3): p. 430-44.
72. Tian, H. B., et al., Efficient differentiation of embryonic stem cells into neurons in glial cell-conditioned medium under attaching conditions. Acta Biochim Biophys Sin (Shanghai), 2005. 37(7): p. 480-7.
73. Kitazawa, A. and N. Shimizu, Differentiation of mouse embryonic stem cells into neurons using conditioned medium of dorsal root ganglia. J Biosci Bioeng, 2005. 100(1): p. 94-9.
74. Zhang, J. Q., et al., Neural differentiation of embryonic stem cells induced by conditioned medium from neural stem cell. Neuroreport, 2006. 17(10): p. 981-6.
75. Yue, F., et al., Induction of midbrain dopaminergic neurons from primate embryonic stem cells by coculture with sertoli cells. Stem Cells, 2006. 24(7): p. 1695-706.
76. Schumacher, A., et al., Staurosporine is a potent activator of neuronal, glial, and “CNS stem cell-like” neurosphere differentiation in murine embryonic stem cells. Mol Cell Neurosci, 2003. 23(4): p. 669-80.
77. Shim, J. W., et al., Enhanced in vitro midbrain dopamine neuron differentiation, dopaminergic function, neurite outgrowth, and 1-methyl-4-phenylpyridium resistance in mouse embryonic stem cells overexpressing Bcl-XL. J Neurosci, 2004. 24(4): p. 843-52.
78. Hirose, M., et al., Differential expression of mRNAs for PACAP and its receptors during neural differentiation of embryonic stem cells. Regul Pept, 2005. 126(1-2): p. 109-13.
79. Chafai, M., et al., PACAP and VIP promote initiation of electrophysiological activity in differentiating embryonic stem cells. Ann N Y Acad Sci, 2006. 1070: p. 185-9.
80. Murashov, A. K., et al., 17beta-Estradiol enhances neuronal differentiation of mouse embryonic stem cells. FEBS Lett, 2004. 569(1-3): p. 165-8.
81. Shin, D. M., et al., Ascorbic acid responsive genes during neuronal differentiation of embryonic stem cells. Neuroreport, 2004. 15(12): p. 1959-63.
82. Levenberg, S., et al., Neurotrophin-induced differentiation of human embryonic stem cells on three-dimensional polymeric scaffolds. Tissue Eng, 2005. 11(3-4): p. 506-12.
83. Itsykson, P., et al., Derivation of neural precursors from human embryonic stem cells in the presence of noggin. Mol Cell Neurosci, 2005. 30(1): p. 24-36.
84. Nasonkin, I. O. and V. E. Koliatsos, Nonhuman sialic acid Neu5Gc is very low in human embryonic stem cell-derived neural precursors differentiated with B27/N2 and noggin: implications for transplantation. Exp Neurol, 2006. 201(2): p. 525-9.
85. Sonntag, K. C., et al., Enhanced yield of neuroepithelial precursors and midbrain-like dopaminergic neurons from human embryonic stem cells using the bone morphogenic protein antagonist noggin. Stem Cells, 2007. 25(2): p. 411-8.
86. Shin, S., et al., Long-term proliferation of human embryonic stem cell-derived neuroepithelial cells using defined adherent culture conditions. Stem Cells, 2006. 24(1): p. 125-38.
87. Kim, D. W., et al., Stromal cell-derived inducing activity, Nurr1, and signaling molecules synergistically induce dopaminergic neurons from mouse embryonic stem cells. Stem Cells, 2006. 24(3): p. 557-67.
88. Hara, A., et al., Intraocular injection of folate antagonist methotrexate induces neuronal differentiation of embryonic stem cells transplanted in the adult mouse retina. Brain Res, 2006. 1085(1): p. 33-42.
89. Lowell, S., et al., Notch promotes neural lineage entry by pluripotent embryonic stem cells. PLoS Biol, 2006. 4(5): p. e121.
90. Yamazoe, H., et al., One-step induction of neurons from mouse embryonic stem cells in serum-free media containing vitamin B12 and heparin. Cell Transplant, 2006. 15(2): p. 135-45.
91. Kim, E., et al., Mu- and kappa-opioids induce the differentiation of embryonic stem cells to neural progenitors. J Biol Chem, 2006. 281(44): p. 33749-60.
92. Lee, D. S., et al., Cyclopamine treatment of human embryonic stem cells followed by culture in human astrocyte medium promotes differentiation into nestin- and GFAP-expressing astrocytic lineage. Life Sci, 2006. 80(2): p. 154-9.
93. Esmaeili, F., et al., Selegiline induces neuronal phenotype and neurotrophins expression in embryonic stem cells. Rejuvenation Res, 2006. 9(4): p. 475-84.
94. Yamada, M., et al., Electrical stimulation modulates fate determination of differentiating embryonic stem cells. Stem Cells, 2007. 25(3): p. 562-70.
95. Gerrard, L., L. Rodgers, and W. Cui, Differentiation of human embryonic stem cells to neural lineages in adherent culture by blocking bone morphogenetic protein signaling. Stem Cells, 2005. 23(9): p. 1234-41.
96. Bjorklund, L. M., et al., Embryonic stem cells develop into functional dopaminergic neurons after transplantation in a Parkinson rat model. Proc Natl Acad Sci USA, 2002. 99(4): p. 2344-9.
97. Kawasaki, H., et al., Generation of dopaminergic neurons and pigmented epithelia from primate ES cells by stromal cell-derived inducing activity. Proc Natl Acad Sci USA, 2002. 99(3): p. 1580-5.
98. Freed, C. R., Will embryonic stem cells be a useful source of dopamine neurons for transplant into patients with Parkinson's disease? Proc Natl Acad Sci USA, 2002. 99(4): p. 1755-7.
99. Kim, J. H., et al., Dopamine neurons derived from embryonic stem cells function in an animal model of Parkinson's disease. Nature, 2002. 418(6893): p. 50-6.
100. Sasai, Y., Generation of dopaminergic neurons from embryonic stem cells. J Neurol, 2002. 249 Suppl 2: p. II41-4.
101. Yoshizaki, T., et al., Isolation and transplantation of dopaminergic neurons generated from mouse embryonic stem cells. Neurosci Lett, 2004. 363(1): p. 33-7.
102. Yurek, D. M. and A. Fletcher-Turner, Comparison of embryonic stem cell-derived dopamine neuron grafts and fetal ventral mesencephalic tissue grafts: morphology and function. Cell Transplant, 2004. 13(3): p. 295-306.
103. Kim, D. W., Efficient induction of dopaminergic neurons from embryonic stem cells for application to Parkinson's disease. Yonsei Med J, 2004. 45 Suppl: p. 23-7.
104. Perrier, A. L., et al., Derivation of midbrain dopamine neurons from human embryonic stem cells. Proc Natl Acad Sci USA, 2004. 101(34): p. 12543-8.
105. Zeng, X., et al., Dopaminergic differentiation of human embryonic stem cells. Stem Cells, 2004. 22(6): p. 925-40.
106. Xu, H., et al., Neural precursor cells differentiated from mouse embryonic stem cells relieve symptomatic motor behavior in a rat model of Parkinson's disease. Biochem Biophys Res Commun, 2005. 326(1): p. 115-22.
107. Schulz, T. C., et al., Differentiation of human embryonic stem cells to dopaminergic neurons in serum-free suspension culture. Stem Cells, 2004. 22(7): p. 1218-38.
108. Ben-Hur, T., et al., Transplantation of human embryonic stem cell-derived neural progenitors improves behavioral deficit in Parkinsonian rats. Stem Cells, 2004. 22(7): p. 1246-55.
109. Takagi, Y., et al., Dopaminergic neurons generated from monkey embryonic stem cells function in a Parkinson primate model. J Clin Invest, 2005. 115(1): p. 102-9.
110. Park, C. H., et al., In vitro and in vivo analyses of human embryonic stem cell-derived dopamine neurons. J Neurochem, 2005. 92(5): p. 1265-76.
111. Takagi, Y. and J. Takahashi, Primate embryonic stem cells as a source of dopaminergic neurons: A novel transplantation for Parkinson's disease. Discov Med, 2005. 5(26): p. 219-23.
112. Yan, Y., et al., Directed differentiation of dopaminergic neuronal subtypes from human embryonic stem cells. Stem Cells, 2005. 23(6): p. 781-90.
113. Maxwell, S. L. and M. Li, Midbrain dopaminergic development in vivo and in vitro from embryonic stem cells. J Anat, 2005. 207(3): p. 209-18.
114. Chung, S., et al., Neural precursors derived from embryonic stem cells, but not those from fetal ventral mesencephalon, maintain the potential to differentiate into dopaminergic neurons after expansion in vitro. Stem Cells, 2006. 24(6): p. 1583-93.
115. Wichterle, H., et al., Directed differentiation of embryonic stem cells into motor neurons. Cell, 2002. 110(3): p. 385-97.
116. Chiba, S., et al., Transplantation of motoneuron-enriched neural cells derived from mouse embryonic stem cells improves motor function of hemiplegic mice. Cell Transplant, 2003. 12(5): p. 457-68.
117. Miles, G. B., et al., Functional properties of motoneurons derived from mouse embryonic stem cells. J Neurosci, 2004. 24(36): p. 7848-58.
118. Shin, S., S. Dalton, and S. L. Stice, Human motor neuron differentiation from human embryonic stem cells. Stem Cells Dev, 2005. 14(3): p. 266-9.
119. Ikeda, R., et al., Transplantation of neural cells derived from retinoic acid-treated cynomolgus monkey embryonic stem cells successfully improved motor function of hemiplegic mice with experimental brain injury. Neurobiol Dis, 2005. 20(1): p. 38-48.
120. Singh Roy, N., et al., Enhancer-specified GFP-based FACS purification of human spinal motor neurons from embryonic stem cells. Exp Neurol, 2005. 196(2): p. 224-34.
121. Soundararajan, P., et al., Motoneurons derived from embryonic stem cells express transcription factors and develop phenotypes characteristic of medial motor column neurons. J Neurosci, 2006. 26(12): p. 3256-68.
122. Salli, U., et al., Serotonin neurons derived from rhesus monkey embryonic stem cells: similarities to CNS serotonin neurons. Exp Neurol, 2004. 188(2): p. 351-64.
123. Gao, J., et al., Human neural stem cell-derived cholinergic neurons innervate muscle in motoneuron deficient adult rats. Neuroscience, 2005. 131(2): p. 257-62.
124. Pomp, O., et al., Generation of peripheral sensory and sympathetic neurons and neural crest cells from human embryonic stem cells. Stem Cells, 2005. 23(7): p. 923-30.
125. Li, M., et al., Generation of purified neural precursors from embryonic stem cells by lineage selection. Curr Biol, 1998. 8(17): p. 971-4.
126. O'Shea, K. S., Neuronal differentiation of mouse embryonic stem cells: lineage selection andforced differentiation paradigms. Blood Cells Mol Dis, 2001. 27(3): p. 705-12.
127. Schuldiner, M., et al., Induced neuronal differentiation of human embryonic stem cells. Brain Res, 2001. 913(2): p. 201-5.
128. Carpenter, M. K., et al., Enrichment of neurons and neural precursors from human embryonic stem cells. Exp Neurol, 2001. 172(2): p. 383-97.
129. van Inzen, W. G., et al., Neuronal differentiation of embryonic stem cells. Biochim Biophys Acta, 1996. 1312(1): p. 21-6.
130. Andressen, C., et al., Nestin-specific green fluorescent protein expression in embryonic stem cell-derived neural precursor cells used for transplantation. Stem Cells, 2001. 19(5): p. 419-24.
131. Strubing, C., et al., Differentiation of pluripotent embryonic stem cells into the neuronal lineage in vitro gives rise to mature inhibitory and excitatory neurons. Mech Dev, 1995. 53(2): p. 275-87.
132. Westmoreland, J. J., C. R. Hancock, and B. G. Condie, Neuronal development of embryonic stem cells: a model of GABAergic neuron differentiation. Biochem Biophys Res Commun, 2001. 284(3): p. 674-80.
133. Tsukahara, M., et al., A novel putative transmembrane protein, IZP6, is expressed in neural cells during embryogenesis. Dev Growth Differ, 2001. 43(3): p. 285-93.
134. Broccardo, C., et al., ABCA2 is a marker of neural progenitors and neuronal subsets in the adult rodent brain. J Neurochem, 2006. 97(2): p. 345-55.
135. Strubing, C., A. M. Wobus, and J. Hescheler, [Establishment of an in vitro model system for the differentiation of synaptically coupled neurons from mouse embryonic stem cells]. ALTEX, 1995. 12(3): p. 129-137.
136. Carmeliet, P. and R. K. Jain, Angiogenesis in cancer and other diseases. Nature, 2000. 407(6801): p. 249-57.
137. Folkman, J., The role of angiogenesis in tumor growth. Semin Cancer Biol, 1992. 3(2): p. 65-71.
138. Motz, G. T., et al., Tumor endothelium FasL establishes a selective immune barrier promoting tolerance in tumors. Nat Med, 2014. 20(6): p. 607-15.
139. Facciponte, J. G., et al., Tumor endothelial marker 1-specific DNA vaccination targets tumor vasculature. J Clin Invest, 2014. 124(4): p. 1497-511.
140. Meng, M. B., et al., Enhanced radioresponse with a novel recombinant human endostatin protein via tumor vasculature remodeling: experimental and clinical evidence. Radiother Oncol, 2013. 106(1): p. 130-7.
141. Czarnota, G. J., et al., Tumor radiation response enhancement by acoustical stimulation of the vasculature. Proc Natl Acad Sci USA, 2012. 109(30): p. E2033-41.
142. Peng, F., et al., Recombinant human endostatin normalizes tumor vasculature and enhances radiation response in xenografted human nasopharyngeal carcinoma models. PLoS One, 2012. 7(4): p. e34646.
143. Zawaski, J. A., et al., Effects of irradiation on brain vasculature using an in situ tumor model. Int J Radiat Oncol Biol Phys, 2012. 82(3): p. 1075-82.
144. Truman, J. P., et al., Endothelial membrane remodeling is obligate for anti-angiogenic radiosensitization during tumor radiosurgery. PLoS One, 2010. 5(9).
145. Okaji, Y., et al., Pilot study of anti-angiogenic vaccine using fixed whole endothelium in patients with progressive malignancy after failure of conventional therapy. Eur J Cancer, 2008. 44(3): p. 383-90.
146. Brose, K., et al., Slit proteins bind Robo receptors and have an evolutionarily conserved role in repulsive axon guidance. Cell, 1999. 96(6): p. 795-806.
147. Yadav, S. S. and G. Narayan, Role of ROBO4 signalling in developmental and pathological angiogenesis. Biomed Res Int, 2014. 2014: p. 683025.
148. Zhuang, X., et al., Robo4 vaccines induce antibodies that retard tumor growth. Angiogenesis, 2014.
149. Paprocka, M., et al., CD133 positive progenitor endothelial cell lines from human cord blood. Cytometry A, 2011. 79(8): p. 594-602.
150. Chen, C., et al., Adult endothelial progenitor cells retain hematopoiesis potential. Transplant Proc, 2010. 42(9): p. 3745-9.
151. Smadja, D. M., et al., Increased VEGFR2 expression during human late endothelial progenitor cells expansion enhances in vitro angiogenesis with up-regulation of integrin alpha(6). J Cell Mol Med, 2007. 11(5): p. 1149-61.
152. Rafii, S., et al., Angiogenic factors reconstitute hematopoiesis by recruiting stem cells from bone marrow microenvironment. Ann N Y Acad Sci, 2003. 996: p. 49-60.
153. Hattori, K., et al., Placental growth factor reconstitutes hematopoiesis by recruiting VEGFR1(+) stem cells from bone-marrow microenvironment. Nat Med, 2002. 8(8): p. 841-9.
154. Ziegler, B. L., et al., KDR receptor: a key marker defining hematopoietic stem cells. Science, 1999. 285(5433): p. 1553-8.
155. Yan, H. X., et al., Active immunotherapy for mouse breast cancer with irradiated whole-cell vaccine expressing VEGFR2. Oncol Rep, 2013. 29(4): p. 1510-6.
156. Feng, K., et al., Anti-angiogenesis effect on glioma of attenuated Salmonella typhimurium vaccine strain with flk-1 gene. J Huazhong Univ Sci Technolog Med Sci, 2004. 24(4): p. 389-91.
157. Wada, S., et al., Rationale for antiangiogenic cancer therapy with vaccination using epitope peptides derived from human vascular endothelial growth factor receptor 2. Cancer Res, 2005. 65(11): p. 4939-46.
158. Yan, J., et al., A promising new approach of VEGFR2-based DNA vaccine for tumor immunotherapy. Immunol Lett, 2009. 126(1-2): p. 60-6.
159. Ren, S., et al., Inhibition of tumor angiogenesis in lung cancer by T4 phage surface displaying mVEGFR2 vaccine. Vaccine, 2011. 29(34): p. 5802-11.
160. Wei, Y., et al., Enhancement of DNA vaccine efficacy by targeting the xenogeneic human chorionic gonadotropin, survivin and vascular endothelial growth factor receptor 2 combined tumor antigen to the major histocompatibility complex class II pathway. J Gene Med, 2012. 14(5): p. 353-62.
161. Suzuki, H., et al., Multiple therapeutic peptide vaccines consisting of combined novel cancer testis antigens and anti-angiogenic peptides for patients with non-small cell lung cancer. J Transl Med, 2013. 11: p. 97.
162. Lladser, A., et al., Intradermal DNA electroporation induces survivin-specific CTLs, suppresses angiogenesis and confers protection against mouse melanoma. Cancer Immunol Immunother, 2010. 59(1): p. 81-92.
163. Xiang, R., et al., Oral DNA vaccines target the tumor vasculature and microenvironment and suppress tumor growth and metastasis. Immunol Rev, 2008. 222: p. 117-28.
164. Xiang, R., et al., A DNA vaccine targeting survivin combines apoptosis with suppression of angiogenesis in lung tumor eradication. Cancer Res, 2005. 65(2): p. 553-61.
165. Valdez, Y., M. Maia, and E. M. Conway, CD248: reviewing its role in health and disease. Curr Drug Targets, 2012. 13(3): p. 432-9.
166. Plum, S. M., et al., Generation of a specific immunological response to FGF-2 does not affect wound healing or reproduction. Immunopharmacol Immunotoxicol, 2004. 26(1): p. 29-41.
167. Wei, Y. Q., et al., Immunogene therapy of tumors with vaccine based on Xenopus homologous vascular endothelial growth factor as a model antigen. Proc Natl Acad Sci USA, 2001. 98(20): p. 11545-50.
168. Liu, J. Y., et al., Immunotherapy of tumors with vaccine based on quail homologous vascular endothelial growth factor receptor-2. Blood, 2003. 102(5): p. 1815-23.
169. Su, J. M., et al., Active immunogene therapy of cancer with vaccine on the basis of chicken homologous matrix metalloproteinase-2. Cancer Res, 2003. 63(3): p. 600-7.
170. Tan, G. H., et al., Active immunotherapy of tumors with a recombinant xenogeneic endoglin as a model antigen. Eur J Immunol, 2004. 34(7): p. 2012-21.
171. Jiao, J. G., et al., A plasmid DNA vaccine encoding the extracellular domain of porcine endoglin induces anti-tumour immune response against self-endoglin-related angiogenesis in two liver cancer models. Dig Liver Dis, 2006. 38(8): p. 578-87.
172. Nataraj an, M., et al., Oxidative stress signalling: a potential mediator of tumour necrosis factor alpha-induced genomic instability in primary vascular endothelial cells. Br J Radiol, 2007. 80 Spec No 1: p. S13-22.
173. Hida, K., et al., Heterogeneity of tumor endothelial cells. Cancer Sci, 2013. 104(11): p. 1391-5.
174. Wei, Y. Q., et al., Immunotherapy of tumors with xenogeneic endothelial cells as a vaccine. Nat Med, 2000. 6(10): p. 1160-6.
175. Scappaticci, F. A., et al., Polyclonal antibodies to xenogeneic endothelial cells induce apoptosis and block support of tumor growth in mice. Vaccine, 2003. 21(19-20): p. 2667-77.
176. Tanaka, M., et al., Human umbilical vein endothelial cell vaccine therapy in patients with recurrent glioblastoma. Cancer Sci, 2013. 104(2): p. 200-5.
177. Guan, K., et al., Pluripotency of spermatogonial stem cells from adult mouse testis. Nature, 2006. 440(7088): p. 1199-203.
178. Yamazoe, H. and H. Iwata, Efficient generation of dopaminergic neurons from mouse embryonic stem cells enclosed in hollow fibers. Biomaterials, 2006. 27(28): p. 4871-80.
179. Stockmann, C., et al., The impact of the immune system on tumor: angiogenesis and vascular remodeling. Front Oncol, 2014. 4: p. 69.
180. Hirst, D. G., J. Denekamp, and B. Hobson, Proliferation kinetics of endothelial and tumour cells in three mouse mammary carcinomas. Cell Tissue Kinet, 1982. 15(3): p. 251-61.
181. Denekamp, J. and B. Hobson, Endothelial-cell proliferation in experimental tumours. Br J Cancer, 1982. 46(5): p. 711-20.
182. Hobson, B. and J. Denekamp, Endothelial proliferation in tumours and normal tissues: continuous labelling studies. Br J Cancer, 1984. 49(4): p. 405-13.
183. Folkman, J., Tumor angiogenesis: therapeutic implications. N Engl J Med, 1971. 285(21): p. 1182-6.
184. Folkman, J., Fighting cancer by attacking its blood supply. Sci Am, 1996. 275(3): p. 150-4.
185. Ellis, L. M. and I. J. Fidler, Finding the tumor copycat. Therapy fails, patients don't. Nat Med, 2010. 16(9): p. 974-5.
186. Dudley, A. C., Tumor endothelial cells. Cold Spring Harb Perspect Med, 2012. 2(3): p. a006536.
187. Cines, D. B., et al., Endothelial cells in physiology and in the pathophysiology of vascular disorders. Blood, 1998. 91(10): p. 3527-61.
188. Nagy, J. A., A. M. Dvorak, and H. F. Dvorak, VEGF-A and the induction of pathological angiogenesis. Annu Rev Pathol, 2007. 2: p. 251-75.
189. Jain, R. K., Determinants of tumor blood flow: a review. Cancer Res, 1988. 48(10): p. 2641-58.
190. Less, J. R., et al., Microvascular architecture in a mammary carcinoma: branching patterns and vessel dimensions. Cancer Res, 1991. 51(1): p. 265-73.
191. Goel, S., et al., Normalization of the vasculature for treatment of cancer and other diseases. Physiol Rev, 2011. 91(3): p. 1071-121.
192. Bussolati, B., et al., Altered angiogenesis and survival in human tumor-derived endothelial cells. FASEB J, 2003. 17(9): p. 1159-61.
193. Grange, C., et al., Isolation and characterization of human breast tumor-derived endothelial cells. Oncol Rep, 2006. 15(2): p. 381-6.
194. Xiong, Y. Q., et al., Human hepatocellular carcinoma tumor-derived endothelial cells manifest increased angiogenesis capability and drug resistance compared with normal endothelial cells. Clin Cancer Res, 2009. 15(15): p. 4838-46.
195. Hida, K. and M. Klagsbrun, A new perspective on tumor endothelial cells: unexpected chromosome and centrosome abnormalities. Cancer Res, 2005. 65(7): p. 2507-10.
196. Taylor, S. M., et al., Angiogenic factor signaling regulates centrosome duplication in endothelial cells of developing blood vessels. Blood, 2010. 116(16): p. 3108-17.
197. Akiyama, K., et al., Tumor endothelial cells acquire drug resistance by MDR1 up-regulation via VEGF signaling in tumor microenvironment. Am J Pathol, 2012. 180(3): p. 1283-93.
198. Griffioen, A. W., Anti-angiogenesis: making the tumor vulnerable to the immune system. Cancer Immunol Immunother, 2008. 57(10): p. 1553-8.
199. Griffioen, A. W., et al., Tumor angiogenesis is accompanied by a decreased inflammatory response of tumor-associated endothelium. Blood, 1996. 88(2): p. 667-73.
200. Griffioen, A. W., et al., Angiogenesis inhibitors overcome tumor induced endothelial cell anergy. Int J Cancer, 1999. 80(2): p. 315-9.
201. Dirkx, A. E., et al., Anti-angiogenesis therapy can overcome endothelial cell anergy and promote leukocyte-endothelium interactions and infiltration in tumors. FASEB J, 2006. 20(6): p. 621-30.
202. Seaman, S., et al., Genes that distinguish physiological and pathological angiogenesis. Cancer Cell, 2007. 11(6): p. 539-54.
203. Gavilondo, J. V., et al., Specific active immunotherapy with a VEGF vaccine in patients with advanced solid tumors. results of the CENTAURO antigen dose escalation phase I clinical trial. Vaccine, 2014. 32(19): p. 2241-50.
204. Katoh, O., et al., Expression of the vascular endothelial growth factor (VEGF) receptor gene, KDR, in hematopoietic cells and inhibitory effect of VEGF on apoptotic cell death caused by ionizing radiation. Cancer Res, 1995. 55(23): p. 5687-92.
205. Hattori, K., et al., Vascular endothelial growth factor and angiopoietin-1 stimulate postnatal hematopoiesis by recruitment of vasculogenic and hematopoietic stem cells. J Exp Med, 2001. 193(9): p. 1005-14.
206. Larrivee, B., et al., Vascular endothelial growth factor receptor-2 induces survival of hematopoietic progenitor cells. J Biol Chem, 2003. 278(24): p. 22006-13.
207. Chen, R., et al., Anti-metastatic effects of DNA vaccine encoding single-chain trimer composed of MHC I and vascular endothelial growth factor receptor 2 peptide. Oncol Rep, 2015. 33(5): p. 2269-76.
208. McKinney, K. A., et al., Effect of a novel DNA vaccine on angiogenesis and tumor growth in vivo. Arch Otolaryngol Head Neck Surg, 2010. 136(9): p. 859-64.
209. Liang, P. H., et al., Construction of a DNA vaccine encoding Flk-1 extracellular domain and C3d fusion gene and investigation of its suppressing effect on tumor growth. Cancer Immunol Immunother, 2010. 59(1): p. 93-101.
210. Xie, K., et al., Anti-tumor effects of a human VEGFR-2-based DNA vaccine in mouse models. Genet Vaccines Ther, 2009. 7: p. 10.
211. Zuo, S. G., et al., Orally administered DNA vaccine delivery by attenuated Salmonella typhimurium targeting fetal liver kinase 1 inhibits murine Lewis lung carcinoma growth and metastasis. Biol Pharm Bull, 2010. 33(2): p. 174-82.
212. Ishizaki, H., et al., Inhibition of tumor growth with antiangiogenic cancer vaccine using epitope peptides derived from human vascular endothelial growth factor receptor 1. Clin Cancer Res, 2006. 12(19): p. 5841-9.
213. Niethammer, A. G., et al., A DNA vaccine against VEGF receptor 2 prevents effective angiogenesis and inhibits tumor growth. Nat Med, 2002. 8(12): p. 1369-75.
214. Li, Y., et al., Active immunization against the vascular endothelial growth factor receptor flk1 inhibits tumor angiogenesis and metastasis. J Exp Med, 2002. 195(12): p. 1575-84.
215. Seavey, M. M., et al., An anti-vascular endothelial growth factor receptor 2/fetal liver kinase-1 Listeria monocytogenes anti-angiogenesis cancer vaccine for the treatment of primary and metastatic Her-2/neu+ breast tumors in a mouse model. J Immunol, 2009. 182(9): p. 5537-46.
216. Miyazawa, M., et al., Phase I clinical trial using peptide vaccine for human vascular endothelial growth factor receptor 2 in combination with gemcitabine for patients with advanced pancreatic cancer. Cancer Sci, 2010. 101(2): p. 433-9.
217. Okamoto, I., et al., Clinical phase I study of elpamotide, a peptide vaccine for vascular endothelial growth factor receptor 2, in patients with advanced solid tumors. Cancer Sci, 2012. 103(12): p. 2135-8.
218. Hayashi, H., et al., Phase I trial of OTS11101, an anti-angiogenic vaccine targeting vascular endothelial growth factor receptor 1 in solid tumor. Cancer Sci, 2013. 104(1): p. 98-104.
219. Magnusson, P. U., et al., Fibroblast growth factor receptor-1 expression is required for hematopoietic but not endothelial cell development. Arterioscler Thromb Vase Biol, 2005. 25(5): p. 944-9.
220. Burger, P. E., et al., Fibroblast growth factor receptor-1 is expressed by endothelial progenitor cells. Blood, 2002. 100(10): p. 3527-35.
221. He, Q. M., et al., Inhibition of tumor growth with a vaccine based on xenogeneic homologous fibroblast growth factor receptor-1 in mice. J Biol Chem, 2003. 278(24): p. 21831-6.
222. Zheng, S. J., et al., Synergistic anti-tumor effect of recombinant chicken fibroblast growth factor receptor-1-mediated anti-angiogenesis and low-dose gemcitabine in a mouse colon adenocarcinoma model. World J Gastroenterol, 2007. 13(17): p. 2484-9.
223. Li, M., et al., bFGF peptide combined with the pVAX-8CpG plasmid as adjuvant is a novel anticancer vaccine inducing effective immune responses against Lewis lung carcinoma. Mol Med Rep, 2012. 5(3): p. 625-30.
224. Plum, S. M., et al., Administration of a liposomal FGF-2 peptide vaccine leads to abrogation of FGF-2-mediated angiogenesis and tumor development. Vaccine, 2000. 19(9-10): p. 1294-303.
225. Liu, Z., F. Wang, and X. Chen, Integrin alpha(v)beta(3)-Targeted Cancer Therapy. Drug Dev Res, 2008. 69(6): p. 329-339.
226. Wilder, R. L., Integrin alpha V beta 3 as a target for treatment of rheumatoid arthritis and related rheumatic diseases. Ann Rheum Dis, 2002. 61 Suppl 2: p. ii96-9.
227. McNeel, D. G., et al., Phase I trial of a monoclonal antibody specific for alphavbeta3 integrin (MEDI-522) in patients with advanced malignancies, including an assessment of effect on tumor perfusion. Clin Cancer Res, 2005. 11(21): p. 7851-60.
228. Bratt, A., et al., Angiomotin belongs to a novel protein family with conserved coiled-coil and PDZ binding domains. Gene, 2002. 298(1): p. 69-77.
229. Holmgren, L., et al., A DNA vaccine targeting angiomotin inhibits angiogenesis and suppresses tumor growth. Proc Natl Acad Sci USA, 2006. 103(24): p. 9208-13.
230. Arigoni, M., et al., A vaccine targeting angiomotin induces an antibody response which alters tumor vessel permeability and hampers the growth of established tumors. Angiogenesis, 2012. 15(2): p. 305-16.
231. Duff, S. E., et al., CD105 is important for angiogenesis: evidence and potential applications. FASEB J, 2003. 17(9): p. 984-92.
232. Basilio-de-Oliveira, R. P. and V. L. Nunes Pannain, Prognostic angiogenic markers (endoglin, VEGF, CD31) and tumor cell proliferation (Ki67) for gastrointestinal stromal tumors. World J Gastroenterol, 2015. 21(22): p. 6924-30.
233. Svatek, R. S., et al., Preoperative plasma endoglin levels predict biochemical progression after radical prostatectomy. Clin Cancer Res, 2008. 14(11): p. 3362-6.
234. Huang, F. Y., et al., Bacterial surface display of endoglin by antigen 43 induces antitumor effectiveness via bypassing immunotolerance and inhibition of angiogenesis. Int J Cancer, 2014. 134(8): p. 1981-90.
235. Jarosz, M., et al., Therapeutic antitumor potential of endoglin-based DNA vaccine combined with immunomodulatory agents. Gene Ther, 2013. 20(3): p. 262-73.
236. Ryan, B. M., N. O'Donovan, and M. J. Duffy, Survivin: a new target for anti-cancer therapy. Cancer Treat Rev, 2009. 35(7): p. 553-62.
237. Lladser, A., et al., Naked DNA immunization as an approach to target the generic tumor antigen survivin induces humoral and cellular immune responses in mice. Immunobiology, 2006. 211(1-2): p. 11-27.
238. Park, K. W., et al., Robo4 is a vascular-specific receptor that inhibits endothelial migration. Dev Biol, 2003. 261(1): p. 251-67.
239. Bedell, V. M., et al., roundabout4 is essential for angiogenesis in vivo. Proc Natl Acad Sci USA, 2005. 102(18): p. 6373-8.
240. Jones, C. A., et al., Robo4 stabilizes the vascular network by inhibiting pathologic angiogenesis and endothelial hyperpermeability. Nat Med, 2008. 14(4): p. 448-53.
241. Seth, P., et al., Magic roundabout, a tumor endothelial marker: expression and signaling. Biochem Biophys Res Commun, 2005. 332(2): p. 533-41.
242. Cai, H., et al., Roundabout 4 regulates blood-tumor barrier permeability through the modulation of ZO-1, Occludin, and Claudin-5 expression. J Neuropathol Exp Neurol, 2015. 74(1): p. 25-37.
243. Zhuang, X., et al., Robo4 vaccines induce antibodies that retard tumor growth. Angiogenesis, 2015. 18(1): p. 83-95.
244. Acevedo, L. M., S. M. Weis, and D. A. Cheresh, Robo4 counteracts VEGF signaling. Nat Med, 2008. 14(4): p. 372-3.
245. Lou, Y. Y., et al., Immunogene therapy of tumors with a vaccine based on the ligand-binding domain of chicken homologous integrin beta3. Immunol Invest, 2002. 31(1): p. 51-69.
246. Daly, C., et al., Angiopoietin-2 functions as a Tie2 agonist in tumor models, where it limits the effects of VEGF inhibition. Cancer Res, 2013. 73(1): p. 108-18.
247. Niu, G., et al., Constitutive Stat3 activity up-regulates VEGF expression and tumor angiogenesis. Oncogene, 2002. 21(13): p. 2000-8.
248. Arteaga, C. L., The epidermal growth factor receptor: from mutant oncogene in nonhuman cancers to therapeutic target in human neoplasia. J Clin Oncol, 2001. 19(18 Suppl): p. 32S-40S.
249. Fontanini, G., et al., Evaluation of epidermal growth factor-related growth factors and receptors and of neoangiogenesis in completely resected stage I-IIIA non-small-cell lung cancer: amphiregulin and microvessel count are independent prognostic indicators of survival. Clin Cancer Res, 1998. 4(1): p. 241-9.
250. Grandis, J. R., et al., Normalization of EGFR mRNA levels following restoration of wild-type p53 in a head and neck squamous cell carcinoma cell line. Int J Oncol, 1998. 13(2): p. 375-8.
251. Garcia, B., et al., Effective inhibition of the epidermal growth factor/epidermal growth factor receptor binding by anti-epidermal growth factor antibodies is related to better survival in advanced non-small-cell lung cancer patients treated with the epidermal growth factor cancer vaccine. Clin Cancer Res, 2008. 14(3): p. 840-6.
252. Fernandez Lorente, A., et al., Effect of blockade of the EGF system on wound healing in patients vaccinated with CIMAvax®EGF. World J Surg Oncol, 2013. 11: p. 275.
253. Fu, C., et al., Identification of a novel membrane protein, HP59, with therapeutic potential as a target of tumor angiogenesis. Clin Cancer Res, 2001. 7(12): p. 4182-94.
254. Xue, Y., et al., PDGF-BB modulates hematopoiesis and tumor angiogenesis by inducing erythropoietin production in stromal cells. Nat Med, 2012. 18(1): p. 100-10.
255. Pietras, K., et al., Functions of paracrine PDGF signaling in the proangiogenic tumor stroma revealed by pharmacological targeting. PLoS Med, 2008. 5(1): p. e19.
256. Falcon, B. L., et al., Increased vascular delivery and efficacy of chemotherapy after inhibition of platelet-derived growth factor-B. Am J Pathol, 2011. 178(6): p. 2920-30.
257. Hosaka, K., et al., Tumour PDGF-BB expression levels determine dual effects of anti-PDGF drugs on vascular remodelling and metastasis. Nat Commun, 2013. 4: p. 2129.
258. Kaplan, C. D., et al., A novel DNA vaccine encoding PDGFRbeta suppresses growth and dissemination of murine colon, lung and breast carcinoma. Vaccine, 2006. 24(47-48): p. 6994-7002.
259. Brady, J., et al., Human endosialin (tumor endothelial marker 1) is abundantly expressed in highly malignant and invasive brain tumors. J Neuropathol Exp Neurol, 2004. 63(12): p. 1274-83.
260. Rouleau, C., et al., Endosialin protein expression and therapeutic target potential in human solid tumors: sarcoma versus carcinoma. Clin Cancer Res, 2008. 14(22): p. 7223-36.
261. Carson-Walter, E. B., et al., Characterization of TEM1/endosialin in human and murine brain tumors. BMC Cancer, 2009. 9: p. 417.
262. Bagley, R. G., Endosialin: from vascular target to biomarker for human sarcomas. Biomark Med, 2009. 3(5): p. 589-604.
263. MacFadyen, J. R., et al., Endosialin (TEM1, CD248) is a marker of stromal fibroblasts and is not selectively expressed on tumour endothelium. FEBS Lett, 2005. 579(12): p. 2569-75.
264. Christian, S., et al., Endosialin (Tem) is a marker of tumor-associated myofibroblasts and tumor vessel-associated mural cells. Am J Pathol, 2008. 172(2): p. 486-94.
265. St Croix, B., et al., Genes expressed in human tumor endothelium. Science, 2000. 289(5482): p. 1197-202.
266. Felicetti, P., et al., Tumor endothelial marker 8 enhances tumor immunity in conjunction with immunization against differentiation Ag. Cytotherapy, 2007. 9(1): p. 23-34.
267. Ruan, Z., et al., DNA vaccine against tumor endothelial marker 8 inhibits tumor angiogenesis and growth. J Immunother, 2009. 32(5): p. 486-91.
268. Yang, X., H. Zhu, and Z. Hu, Dendritic cells transduced with TEM8 recombinant adenovirus prevents hepatocellular carcinoma angiogenesis and inhibits cells growth. Vaccine, 2010. 28(43): p. 7130-5.
269. Liu, P., et al., Anti-tumor angiogenesis effect of genetic fusion vaccine encoding murine beta-defensin 2 and tumor endothelial marker-8 in a CT-26 murine colorectal carcinoma model. Int J Clin Exp Med, 2015. 8(3): p. 4744-52.
270. Okaji, Y., et al., Vaccination with autologous endothelium inhibits angiogenesis and metastasis of colon cancer through autoimmunity. Cancer Sci, 2004. 95(1): p. 85-90.
271. Yoshiura, K., et al., Inhibition of B16 melanoma growth and metastasis in C57BL mice by vaccination with a syngeneic endothelial cell line. J Exp Clin Cancer Res, 2009. 28: p. 13.
272. Scappaticci, F. A. and G. P. Nolan, Induction of anti-tumor immunity in mice using a syngeneic endothelial cell vaccine. Anticancer Res, 2003. 23(2B): p. 1165-72.
273. Corsini, E., et al., Immunotherapy with bovine aortic endothelial cells in subcutaneous and intracerebral glioma models in rats: effects on survival time, tumor growth, and tumor neovascularization. Cancer Immunol Immunother, 2004. 53(11): p. 955-62.
274. Zhang, W., J. N. Liu, and X. Y. Tan, Vaccination with xenogeneic tumor endothelial proteins isolated in situ inhibits tumor angiogenesis and spontaneous metastasis. Int J Cancer, 2009. 125(1): p. 124-32.
275. Catalona, W. J., et al., Identification of complement-receptor lymphocytes (B cells) in lymph nodes and tumor infiltrates. J Urol, 1975. 114(6): p. 915-21.
276. Wolf, G. T., et al., Lymphocyte subpopulations infiltrating squamous carcinomas of the head and neck: correlations with extent of tumor and prognosis. Otolaryngol Head Neck Surg, 1986. 95(2): p. 142-52.
277. Holmes, E. C., Immunology of tumor infiltrating lymphocytes. Ann Surg, 1985. 201(2): p. 158-63.
278. Lipponen, P. K., et al., Tumour infiltrating lymphocytes as an independent prognostic factor in transitional cell bladder cancer. Eur J Cancer, 1992. 29A(1): p. 69-75.
279. Di Giorgio, A., et al., The influence of tumor lymphocytic infiltration on long term survival of surgically treated colorectal cancer patients. Int Surg, 1992. 77(4): p. 256-60.
280. Kawata, A., et al., Tumor-infiltrating lymphocytes and prognosis of hepatocellular carcinoma. Jpn J Clin Oncol, 1992. 22(4): p. 256-63.
281. Furihata, M., et al., Prognostic significance of simultaneous infiltration of HLA-DR-positive dendritic cells and tumor infiltrating lymphocytes into human esophageal carcinoma. Tohoku J Exp Med, 1993. 169(3): p. 187-95.
282. Sarioglu, T., et al., The effect of lymphocytic infiltration on clinical survival in cancer of the tongue. Eur Arch Otorhinolaryngol, 1994. 251(6): p. 366-9.
283. Slootweg, P. J., et al., Lymphocytes at tumor margins in patients with head and neck cancer. Relationship with tumor size, human lymphocyte antigen molecules, and metastasis. Int J Oral Maxillofac Surg, 1994. 23(5): p. 286-9.
284. Mihm, M. C., Jr., C. G. Clemente, and N. Cascinelli, Tumor infiltrating lymphocytes in lymph node melanoma metastases: a histopathologic prognostic indicator and an expression of local immune response. Lab Invest, 1996. 74(1): p. 43-7.
285. Clemente, C. G., et al., Prognostic value of tumor infiltrating lymphocytes in the vertical growth phase of primary cutaneous melanoma. Cancer, 1996. 77(7): p. 1303-10.
286. Hakansson, A., et al., Tumour-infiltrating lymphocytes in metastatic malignant melanoma and response to interferon alpha treatment. Br J Cancer, 1996. 74(5): p. 670-6.
287. Eerola, A. K., Y. Soini, and P. Paakko, Tumour infiltrating lymphocytes in relation to tumour angiogenesis, apoptosis and prognosis in patients with large cell lung carcinoma. Lung Cancer, 1999. 26(2): p. 73-83.
288. Prince, M. E. P., et al., Evaluation of the immunogenicity of ALDH(high) human head and neck squamous cell carcinoma cancer stem cells in vitro. Oral Oncol, 2016. 59: p. 30-42.
289. Huang, Z., et al., Correlation of cancer stem cell markers and immune cell markers in resected non-small cell lung cancer. J Cancer, 2017. 8(16): p. 3190-3197.
290. Codd, A. S., et al., Cancer stem cells as targets for immunotherapy. Immunology, 2017.
291. Rapp, C., et al., Identification of T cell target antigens in glioblastoma stem-like cells using an integrated proteomics-based approach in patient specimens. Acta Neuropathol, 2017. 134(2): p. 297-316.
292. Maccalli, C., G. Parmiani, and S. Ferrone, Immunomodulating and Immunoresistance Properties of Cancer-Initiating Cells: Implications for the Clinical Success of Immunotherapy. Immunol Invest, 2017. 46(3): p. 221-238.
293. Friedman, H. S., et al., Bevacizumab alone and in combination with irinotecan in recurrent glioblastoma. J Clin Oncol, 2009. 27(28): p. 4733-40.
294. Kingsley, D. P., An interesting case of possible abscopal effect in malignant melanoma. Br J Radiol, 1975. 48(574): p. 863-6.
295. Ohba, K., et al., Abscopal regression of hepatocellular carcinoma after radiotherapy for bone metastasis. Gut, 1998. 43(4): p. 575-7.
296. Takaya, M., et al., Abscopal effect of radiation on toruliform para-aortic lymph node metastases of advanced uterine cervical carcinoma—a case report. Anticancer Res, 2007. 27(1B): p. 499-503.
297. Okuma, K., et al., Abscopal effect of radiation on lung metastases of hepatocellular carcinoma: a case report. J Med Case Rep, 2011. 5: p. 111.
298. Ghodadra, A., et al., Abscopal Effects and Yttrium-90 Radioembolization. Cardiovasc Intervent Radiol, 2016. 39(7): p. 1076-80.
299. Lakshmanagowda, P. B., et al., Abscopal effect in a patient with chronic lymphocytic leukemia during radiation therapy: a case report. Cases J, 2009. 2: p. 204.
300. Aalbers, A. M., et al., The beneficial local and abscopal effect of splenic irradiation in frail patients with chronic lymphocytic leukaemia. Neth J Med, 2016. 74(3): p. 122-9.
301. Cotter, S. E., et al., Abscopal effect in a patient with metastatic Merkel cell carcinoma following radiation therapy: potential role of induced antitumor immunity. Arch Dermatol, 2011. 147(7): p. 870-2.
302. Postow, M. A., et al., Immunologic correlates of the abscopal effect in a patient with melanoma. N Engl J Med, 2012. 366(10): p. 925-31.
303. Stamell, E. F., et al., The abscopal effect associated with a systemic anti-melanoma immune response. Int J Radiat Oncol Biol Phys, 2013. 85(2): p. 293-5.
304. Grimaldi, A. M., et al., Abscopal effects of radiotherapy on advanced melanoma patients who progressed after ipilimumab immunotherapy. Oncoimmunology, 2014. 3: p. e28780.
305. Ishiyama, H., et al., Spontaneous regression of thoracic metastases while progression of brain metastases after stereotactic radiosurgery and stereotactic body radiotherapy for metastatic renal cell carcinoma: abscopal effect prevented by the blood-brain barrier? Clin Genitourin Cancer, 2012. 10(3): p. 196-8.
306. Saba, R., N. Saleem, and D. Peace, Long-term survival consequent on the abscopal effect in a patient with multiple myeloma. BMJ Case Rep, 2016. 2016.
307. Ungaro, A., et al., Successful palliative approach with high-intensity focused ultrasound in a patient with metastatic anaplastic pancreatic carcinoma: a case report. Ecancermedicalscience, 2016. 10: p. 635.
308. Shi, F., et al., Abscopal effect of metastatic pancreatic cancer after local radiotherapy and granulocyte-macrophage colony-stimulating factor therapy. Cancer Biol Ther, 2017. 18(3): p. 137-141.
309. Demaria, S., et al., The TLR7 agonist imiquimod as an adjuvant for radiotherapy-elicited in situ vaccination against breast cancer. Oncoimmunology, 2013. 2(10): p. e25997.
310. Leung, H. W., et al., Abscopal effect of radiation on bone metastases of breast cancer: A case report. Cancer Biol Ther, 2018. 19(1): p. 20-24.
311. Van de Walle, M., et al., Abscopal effect in metastatic renal cell carcinoma. Acta Clin Belg, 2017. 72(4): p. 245-249.
312. Desar, I. M., et al., Abscopal effect of radiotherapy in a patient with metastatic diffuse-type giant cell tumor. Acta Oncol, 2016. 55(12): p. 1510-1512.
313. Cong, Y., et al., Abscopal regression following SABR for non-small-cell-lung cancer: A case report. Cancer Biol Ther, 2017. 18(1): p. 1-3.
314. Demaria, S., et al., Ionizing radiation inhibition of distant untreated tumors (abscopal effect) is immune mediated. Int J Radiat Oncol Biol Phys, 2004. 58(3): p. 862-70.
315. Van der Meeren, A., et al., Abdominal radiation exposure elicits inflammatory responses and abscopal effects in the lungs of mice. Radiat Res, 2005. 163(2): p. 144-52.
316. Shiraishi, K., et al., Enhancement of antitumor radiation efficacy and consistent induction of the abscopal effect in mice by ECI301, an active variant of macrophage inflammatory protein-1alpha. Clin Cancer Res, 2008. 14(4): p. 1159-66.
317. Dewan, M. Z., et al., Fractionated but not single-dose radiotherapy induces an immune-mediated abscopal effect when combined with anti-CTLA-4 antibody. Clin Cancer Res, 2009. 15(17): p. 5379-88.
318. Wu, L., et al., Targeting the inhibitory receptor CTLA-4 on T cells increased abscopal effects in murine mesothelioma model. Oncotarget, 2015. 6(14): p. 12468-80.
319. Vanpouille-Box, C., et al., In situ vaccination by radiotherapy to improve responses to anti-CTLA-4 treatment. Vaccine, 2015. 33(51): p. 7415-7422.
320. Seyedin, S. N., et al., Strategies for combining immunotherapy with radiation for anticancer therapy. Immunotherapy, 2015. 7(9): p. 967-980.
321. Chandra, R. A., et al., A systematic evaluation of abscopal responses following radiotherapy in patients with metastatic melanoma treated with ipilimumab. Oncoimmunology, 2015. 4(11): p. e1046028.
322. Grass, G. D., N. Krishna, and S. Kim, The immune mechanisms of abscopal effect in radiation therapy. Curr Probl Cancer, 2016. 40(1): p. 10-24.
323. Lock, M., et al., Abscopal Effects: Case Report and Emerging Opportunities. Cureus, 2015. 7(10): p. e344.
324. Levy, A., et al., Can immunostimulatory agents enhance the abscopal effect of radiotherapy? Eur J Cancer, 2016. 62: p. 36-45.
325. Park, S. S., et al., PD-1 Restrains Radiotherapy-Induced Abscopal Effect. Cancer Immunol Res, 2015. 3(6): p. 610-9.
326. Vanpouille-Box, C., et al., TGFbeta Is a Master Regulator of Radiation Therapy-Induced Antitumor Immunity. Cancer Res, 2015. 75(11): p. 2232-42.
327. Ma, S. Y., et al., Addition of anti-neu antibody to local irradiation can improve tumor-bearing BALB/c mouse survival through immune-mediated mechanisms. Radiat Res, 2015. 183(3): p. 271-8.
328. Camphausen, K., et al., Radiation abscopal antitumor effect is mediated through p53. Cancer Res, 2003. 63(8): p. 1990-3.
329. Michot, J. M., et al., Abscopal effect in a Hodgkin lymphoma patient treated by an anti-programmed death 1 antibody. Eur J Cancer, 2016. 66: p. 91-4.
330. Kang, J., S. Demaria, and S. Formenti, Current clinical trials testing the combination of immunotherapy with radiotherapy. J Immunother Cancer, 2016. 4: p. 51.
331. Ribeiro Gomes, J., et al., Analysis of the Abscopal Effect With Anti-PD1 Therapy in Patients With Metastatic Solid Tumors. J Immunother, 2016. 39(9): p. 367-372.
332. Ishihara, D., et al., Rationale and evidence to combine radiation therapy and immunotherapy for cancer treatment. Cancer Immunol Immunother, 2017. 66(3): p. 281-298.
333. Takeshima, T., et al., Key role for neutrophils in radiation-induced antitumor immune responses: Potentiation with G-CSF. Proc Natl Acad Sci USA, 2016. 113(40): p. 11300-11305.
334. Ablin, R. J., et al., Precipitating antibody in the sera of patients treated cryosurgically for carcinoma of the prostate. Exp Med Surg, 1969. 27(4): p. 406-10.
335. Faraci, R. P., et al., The effect of curative cryosurgery on the tumor-specific immune response of C57 mice. Cryobiology, 1975. 12(2): p. 175-9.
336. Ikeda, K., Immune response of distant lesions following cryosurgery in the experimental tumor system. Fukushima J Med Sci, 1976. 23(3-4): p. 45-67.
337. Snyder, A. R., Review of radiation-induced bystander effects. Hum Exp Toxicol, 2004. 23(2): p. 87-9.
338. Tang, C., et al., Combining radiation and immunotherapy: a new systemic therapy for solid tumors? Cancer Immunol Res, 2014. 2(9): p. 831-8.
339. Reynders, K., et al., The abscopal effect of local radiotherapy: using immunotherapy to make a rare event clinically relevant. Cancer Treat Rev, 2015. 41(6): p. 503-10.
340. Dong, C., et al., The differential role of human macrophage in triggering secondary bystander effects after either gamma-ray or carbon beam irradiation. Cancer Lett, 2015. 363(1): p. 92-100.
341. Ichim, T. E., et al., Induction of tumor inhibitory anti-angiogenic response through immunization with interferon Gamma primed placental endothelial cells: ValloVax. J Transl Med, 2015. 13: p. 90.
342. Wagner, S. C., et al., Cancer anti-angiogenesis vaccines: Is the tumor vasculature antigenically unique? J Transl Med, 2015. 13: p. 340.
343. Wagner, S. C., et al., Safety of targeting tumor endothelial cell antigens. J Transl Med, 2016. 14: p. 90.
344. Wagner, S. C., et al., Induction and characterization of anti-tumor endothelium immunity elicited by ValloVax therapeutic cancer vaccine. Oncotarget, 2017. 8(17): p. 28595-28613.
345. Zhu, X. Y., et al., Mesenchymal stem cells and endothelial progenitor cells decrease renal injury in experimental swine renal artery stenosis through different mechanisms. Stem Cells, 2013. 31(1): p. 117-25.
346. Lopez-Holgado, N., et al., Short-term endothelial progenitor cell colonies are composed of monocytes and do not acquire endothelial markers. Cytotherapy, 2007. 9(1): p. 14-22.
347. Achyut, B. R. and A. S. Arbab, Myeloid Derived Suppressor Cells: Fuel the Fire. Biochem Physiol, 2014. 3(3): p. e123.
348. Sander, A. L., et al., Systemic transplantation of progenitor cells accelerates wound epithelialization and neovascularization in the hairless mouse ear wound model. J Surg Res, 2011. 165(1): p. 165-70.
349. Fortini, C., et al., Circulating stem cell vary with NYHA stage in heart failure patients. J Cell Mol Med, 2011. 15(8): p. 1726-36.
350. Tardif, K., et al., A phosphorylcholine-modified chitosan polymer as an endothelial progenitor cell supporting matrix. Biomaterials, 2011. 32(22): p. 5046-55.
351. Campioni, D., et al., In vitro characterization of circulating endothelial progenitor cells isolated from patients with acute coronary syndrome. PLoS One, 2013. 8(2): p. e56377.
352. Nowak, W. N., et al., Number of circulating pro-angiogenic cells, growth factor and anti-oxidative gene profiles might be altered in type 2 diabetes with and without diabetic foot syndrome. J Diabetes Investig, 2014. 5(1): p. 99-107.
353. Obtulowicz, P., et al., Induction of Endothelial Phenotype From Wharton's Jelly-Derived MSCs and Comparison of Their Vasoprotective and Neuroprotective Potential With Primary WJ-MSCs in CA1 Hippocampal Region Ex Vivo. Cell Transplant, 2016. 25(4): p. 715-27.
354. Rohde, E., et al., Blood monocytes mimic endothelial progenitor cells. Stem Cells, 2006. 24(2): p. 357-67.
355. Bagley, R. G., et al., Pericytes and endothelial precursor cells: cellular interactions and contributions to malignancy. Cancer Res, 2005. 65(21): p. 9741-50.
356. Finney, M. R., et al., Direct comparison of umbilical cord blood versus bone marrow-derived endothelial precursor cells in mediating neovascularization in response to vascular ischemia. Biol Blood Marrow Transplant, 2006. 12(5): p. 585-93.
357. Neumuller, J., et al., Immunological and ultrastructural characterization of endothelial cell cultures differentiated from human cord blood derived endothelial progenitor cells. Histochem Cell Biol, 2006. 126(6): p. 649-64.
358. Nguyen, V. A., et al., Endothelial cells from cord blood CD133+CD34+ progenitors share phenotypic, functional and gene expression profile similarities with lymphatics. J Cell Mol Med, 2009. 13(3): p. 522-34.
359. Kamei, N., et al., Lnk deletion reinforces the function of bone marrow progenitors in promoting neovascularization and astrogliosis following spinal cord injury. Stem Cells, 2010. 28(2): p. 365-75.
360. Kuliczkowski, W., et al., Endothelial progenitor cells and left ventricle function in patients with acute myocardial infarction: potential therapeutic considerations. Am J Ther, 2012. 19(1): p. 44-50.
361. Damaghi, M. and R. Gillies, Phenotypic changes of acid adapted cancer cells push them toward aggressiveness in their evolution in the tumor microenvironment. Cell Cycle, 2016: p. 0.
362. Lobo, R. C., et al., Glucose Uptake and Intracellular pH in a Mouse Model of Ductal Carcinoma In situ (DCIS) Suggests Metabolic Heterogeneity. Front Cell Dev Biol, 2016. 4: p. 93.
363. An, S., et al., Amino Acid Metabolism Abnormity and Microenvironment Variation Mediated Targeting and Controlled Glioma Chemotherapy. Small, 2016.
364. Avnet, S., et al., Altered pH gradient at the plasma membrane of osteosarcoma cells is a key mechanism of drug resistance. Oncotarget, 2016.
365. Huang, S., et al., Acidic extracellular pH promotes prostate cancer bone metastasis by enhancing PC-3 stem cell characteristics, cell invasiveness and VEGF-induced vasculogenesis of BM-EPCs. Oncol Rep, 2016. 36(4): p. 2025-32.
366. Carnero, A. and M. Lleonart, The hypoxic microenvironment. A determinant of cancer stem cell evolution. Bioessays, 2016. 38 Suppl 1: p. S65-74.
367. Pellegrini, P., et al., Tumor acidosis enhances cytotoxic effects and autophagy inhibition by salinomycin on cancer cell lines and cancer stem cells. Oncotarget, 2016. 7(24): p. 35703-35723.
368. Bohme, I. and A. K. Bosserhoff, Acidic tumor microenvironment in human melanoma. Pigment Cell Melanoma Res, 2016. 29(5): p. 508-23.
369. Gentric, G., V. Mieulet, and F. Mechta-Grigoriou, Heterogeneity in Cancer Metabolism: New Concepts in an Old Field. Antioxid Redox Signal, 2016.
370. Ge, Y., et al., Preferential extension of short telomeres induced by low extracellular pH. Nucleic Acids Res, 2016. 44(17): p. 8086-96.
371. Quail, D. F., et al., The tumor microenvironment underlies acquired resistance to CSF-1R inhibition in gliomas. Science, 2016. 352(6288): p. aad3018.
372. Li, X., et al., The altered glucose metabolism in tumor and a tumor acidic microenvironment associated with extracellular matrix metalloproteinase inducer and monocarboxylate transporters. Oncotarget, 2016. 7(17): p. 23141-55.
373. Zhao, C., et al., Tumor Acidity-Induced Sheddable Polyethylenimine-Poly(trimethylene carbonate)/DNA/Polyethylene Glycol-2,3-Dimethylmaleicanhydride Ternary Complex for Efficient and Safe Gene Delivery. ACS Appl Mater Interfaces, 2016. 8(10): p. 6400-10.
374. Riemann, A., et al., Acidosis Promotes Metastasis Formation by Enhancing Tumor Cell Motility. Adv Exp Med Biol, 2016. 876: p. 215-20.
375. Ravishankar, B., et al., The amino acid sensor GCN2 inhibits inflammatory responses to apoptotic cells promoting tolerance and suppressing systemic autoimmunity. Proc Natl Acad Sci USA, 2015. 112(34): p. 10774-9.
376. Walls, J., L. Sinclair, and D. Finlay, Nutrient sensing, signal transduction and immune responses. Semin Immunol, 2016.
377. Munn, D. H., et al., GCN2 kinase in T cells mediates proliferative arrest and anergy induction in response to indoleamine 2,3-dioxygenase. Immunity, 2005. 22(5): p. 633-42.
378. Ishimura, R., et al., Activation of GCN2 kinase by ribosome stalling links translation elongation with translation initiation. Elife, 2016. 5.
379. Lageix, S., et al., Enhanced interaction between pseudokinase and kinase domains in Gcn2 stimulates eIF2alpha phosphorylation in starved cells. PLoS Genet, 2014. 10(5): p. e1004326.
380. Metz, R., et al., IDO inhibits a tryptophan sufficiency signal that stimulates mTOR: A novel IDO effector pathway targeted by D-1-methyl-tryptophan. Oncoimmunology, 2012. 1(9): p. 1460-1468.
381. Manlapat, A. K., et al., Cell-autonomous control of interferon type I expression by indoleamine 2,3-dioxygenase in regulatory CD19+ dendritic cells. Eur J Immunol, 2007. 37(4): p. 1064-71.
382. Sharma, M. D., et al., Plasmacytoid dendritic cells from mouse tumor-draining lymph nodes directly activate mature Tregs via indoleamine 2,3-dioxygenase. J Clin Invest, 2007. 117(9): p. 2570-82.
383. Forouzandeh, F., et al., Differential immunosuppressive effect of indoleamine 2,3-dioxygenase (IDO) on primary human CD4+ and CD8+ T cells. Mol Cell Biochem, 2008. 309(1-2): p. 1-7.
384. Wek, R. C., H. Y. Jiang, and T. G. Anthony, Coping with stress: eIF2 kinases and translational control. Biochem Soc Trans, 2006. 34(Pt 1): p. 7-11.
385. Xia, X. J., et al., Autophagy mediated by arginine depletion activation of the nutrient sensor GCN2 contributes to interferon-gamma-induced malignant transformation of primary bovine mammary epithelial cells. Cell Death Discov, 2016. 2: p. 15065.
386. Xia, X., et al., Arginine Supplementation Recovered the IFN-gamma-Mediated Decrease in Milk Protein and Fat Synthesis by Inhibiting the GCN2/eIF2alpha Pathway, Which Induces Autophagy in Primary Bovine Mammary Epithelial Cells. Mol Cells, 2016. 39(5): p. 410-7.
387. Vynnytska-Myronovska, B. O., et al., Arginine starvation in colorectal carcinoma cells: Sensing, impact on translation control and cell cycle distribution. Exp Cell Res, 2016. 341(1): p. 67-74.
388. Marion, V., et al., Arginine deficiency causes runting in the suckling period by selectively activating the stress kinase GCN2. J Biol Chem, 2011. 286(11): p. 8866-74.
389. Rodriguez, P. C., D. G. Quiceno, and A. C. Ochoa, L-arginine availability regulates T-lymphocyte cell-cycle progression. Blood, 2007. 109(4): p. 1568-73.
390. Bunpo, P., et al., The eIF2 kinase GCN2 is essential for the murine immune system to adapt to amino acid deprivation by asparaginase. J Nutr, 2010. 140(11): p. 2020-7.
391. Bunpo, P., et al., GCN2 protein kinase is required to activate amino acid deprivation responses in mice treated with the anti-cancer agent L-asparaginase. J Biol Chem, 2009. 284(47): p. 32742-9.
392. Balasubramanian, M. N., E. A. Butterworth, and M. S. Kilberg, Asparagine synthetase: regulation by cell stress and involvement in tumor biology. Am J Physiol Endocrinol Metab, 2013. 304(8): p. E789-99.
393. Wilson, G. J., et al., The eukaryotic initiation factor 2 kinase GCN2 protects against hepatotoxicity during asparaginase treatment. Am J Physiol Endocrinol Metab, 2013. 305(9): p. E1124-33.
394. Ye, J., et al., GCN2 sustains mTORC1 suppression upon amino acid deprivation by inducing Sestrin2. Genes Dev, 2015. 29(22): p. 2331-6.
395. Reinert, R. B., et al., Role of glutamine depletion in directing tissue-specific nutrient stress responses to L-asparaginase. J Biol Chem, 2006. 281(42): p. 31222-33.
396. Drogat, B., et al., Acute L-glutamine deprivation compromises VEGF-a upregulation in A549/8 human carcinoma cells. J Cell Physiol, 2007. 212(2): p. 463-72.
397. Peters, O. M., M. Ghasemi, and R. H. Brown, Jr., Emerging mechanisms of molecular pathology in ALS. J Clin Invest, 2015. 125(5): p. 1767-79.
398. Ling, S. C., M. Polymenidou, and D. W. Cleveland, Converging mechanisms in ALS and FTD: disrupted RNA and protein homeostasis. Neuron, 2013. 79(3): p. 416-38.
399. O'Toole, O., et al., Epidemiology and clinical features of amyotrophic lateral sclerosis in Ireland between 1995 and 2004. J Neurol Neurosurg Psychiatry, 2008. 79(1): p. 30-2.
400. Armon, C., Sports and trauma in amyotrophic lateral sclerosis revisited. J Neurol Sci, 2007. 262(1-2): p. 45-53.
401. Alonso, A., et al., Incidence and lifetime risk of motor neuron disease in the United Kingdom: a population-based study. Eur J Neurol, 2009. 16(6): p. 745-51.
402. Sreedharan, J. and R. H. Brown, Jr., Amyotrophic lateral sclerosis: Problems and prospects. Ann Neurol, 2013. 74(3): p. 309-16.
403. Mazzini, L., et al., Stem cell therapy in amyotrophic lateral sclerosis: a methodological approach in humans. Amyotroph Lateral Scler Other Motor Neuron Disord, 2003. 4(3): p. 158-61.
404. Pittenger, M. F., et al., Multilineage potential of adult human mesenchymal stem cells. Science, 1999. 284(5411): p. 143-7.
405. Mazzini, L., et al., Autologous mesenchymal stem cells: clinical applications in amyotrophic lateral sclerosis. Neurol Res, 2006. 28(5): p. 523-6.
406. Mazzini, L., et al., Mesenchymal stem cell transplantation in amyotrophic lateral sclerosis: A Phase I clinical trial. Exp Neurol, 2010. 223(1): p. 229-37.
407. Mazzini, L., et al., Mesenchymal stromal cell transplantation in amyotrophic lateral sclerosis: a long-term safety study. Cytotherapy, 2012. 14(1): p. 56-60.
408. Oh, K. W., et al., Phase I trial of repeated intrathecal autologous bone marrow-derived mesenchymal stromal cells in amyotrophic lateral sclerosis. Stem Cells Transl Med, 2015. 4(6): p. 590-7.
409. Rushkevich, Y. N., et al., The Use of Autologous Mesenchymal Stem Cells for Cell Therapy of Patients with Amyotrophic Lateral Sclerosis in Belarus. Bull Exp Biol Med, 2015. 159(4): p. 576-81.
410. Baek, W., et al., Stem cell transplantation into the intraventricular space via an Ommaya reservoir in a patient with amyotrophic lateral sclerosis. J Neurosurg Sci, 2012. 56(3): p. 261-3.
411. Sundaresan, N. and N. D. Suite, Optimal use of the Ommaya reservoir in clinical oncology. Oncology (Williston Park), 1989. 3(12): p. 15-22; discussion 23.
412. Quattrocchi, K. B., et al., Pilot study of local autologous tumor infiltrating lymphocytes for the treatment of recurrent malignant gliomas. J Neurooncol, 1999. 45(2): p. 141-57.
413. Jung, G., et al., Local immunotherapy of glioma patients with a combination of 2 bispecific antibody fragments and resting autologous lymphocytes: evidence for in situ t-cell activation and therapeutic efficacy. Int J Cancer, 2001. 91(2): p. 225-30.
414. Clemons-Miller, A. R., et al., Intrathecal cytotoxic T-cell immunotherapy for metastatic leptomeningeal melanoma. Clin Cancer Res, 2001. 7(3 Suppl): p. 917s-924s.
415. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
416. Berry, J. D., et al., NurOwn, phase 2, randomized, clinical trial in patients with ALS: Safety, clinical, and biomarker results. Neurology, 2019. 93(24): p. e2294-e2305.
417. Ferensztajn-Rochowiak, E. and J. K. Rybakowski, The effect of lithium on hematopoietic, mesenchymal and neural stem cells. Pharmacol Rep, 2016. 68(2): p. 224-30.
418. Yoneyama, M., et al., Lithium promotes neuronal repair and ameliorates depression-like behavior following trimethyltin-induced neuronal loss in the dentate gyrus. PLoS One, 2014. 9(2): p. e87953.
419. Boku, S., et al., Valproate recovers the inhibitory effect of dexamethasone on the proliferation of the adult dentate gyrus-derived neural precursor cells via GSK-3beta and beta-catenin pathway. Eur J Pharmacol, 2014. 723: p. 425-30.
420. Belayev, L., et al., A novel neurotrophic therapeutic strategy for experimental stroke. Brain Res, 2009. 1280: p. 117-23.
421. Boll, M. C., et al., Clinical and biological changes under treatment with lithium carbonate and valproic acid in sporadic amyotrophic lateral sclerosis. J Neurol Sci, 2014. 340(1-2): p. 103-8.

1. A method of treating cancer and/or overcoming cancer associated immune suppression comprising the steps of: a) selecting a patient suffering from a tumor; and b) administering to said patient a neuronal population grown ex vivo.

2. The method of claim 1, wherein said patient is selected based on amount of immune cell infiltration into said tumor.

3. The method of claim 2, wherein said cellular infiltration comprises tumor infiltrating lymphocytes.

4. The method of claim 1, wherein said cancer is treated by administration of neuronal or neuronal-like cells.

5. The method of claim 4, wherein said neuronal or neuronal-like cells are administered intratumorally.

6. The method of claim 5, wherein said neuronal or neuronal-like cells are administered in an immature state.

7. The method of claim 6, wherein said immature neuronal or neuronal-like cells are allowed to differentiate intratumorally.

8. The method of claim 7, wherein said immature neuronal or neuronal-like cells are generated from hematopoietic stem cells.

9. The method of claim 8, wherein said hematopoietic stem cells are capable of generating leukocytic, lymphocytic, thrombocytic and erythrocytic cells when transplanted into an immunodeficient animal.

10. The method of claim 9, wherein said hematopoietic stem cell is non-adherent to plastic.

11. The method of claim 9, wherein said hematopoietic stem cell is adherent to plastic.

12. The method of claim 9, wherein said hematopoietic stem cell is exposed to hyperthermia.

13. The method of claim 9, wherein said hematopoietic stem cell expresses interleukin-3 receptor.

14. The method of claim 9, wherein said hematopoietic stem cell expresses interleukin-1 receptor.

15. The method of claim 9, wherein said hematopoietic stem cell expresses c-met.

16. The method of claim 9, wherein said hematopoietic stem cell expresses mpl.

17. The method of claim 7, wherein said neuronal or neuronal like immature cells are generated from mesenchymal stem cells.

18. The method of claim 7, wherein said mesenchymal stem cells are naturally occurring mesenchymal stem cells.

19. The method of claim 7, wherein said mesenchymal stem cells are generated in vitro.

20. The method of claim 7, wherein said naturally occurring mesenchymal stem cells are tissue derived.
